(19) United States
(12) Patent Application Publication
(10) Pub. No.: US 2025/0017889 A1
(43) Pub. Date: Jan. 16, 2025

(54) INHIBITORS OF P1B-TYPE ATPASES

(71) Applicant: THE CURATORS OF THE UNIVERSITY OF MISSOURI, Columbia, MO (US)

(72) Inventors: Michael Petris, Columbia, MO (US); Kamlendra Singh, Columbia, MO (US); Vinit Shanbhag, Columbia, MO (US)

(21) Appl. No.: 18/701,297

(22) PCT Filed: Oct. 14, 2022
(86) PCT No.: PCT/US2022/078099

§ 371 Date: Apr. 15, 2024

Related U.S. Application Data
(60) Provisional application No. 63/262,611, filed on Oct. 15, 2021.

(51) Int. Cl. A61K 31/265; A61K 33/34; A61K 33/38; A61P 35/00; C07C 327/40

(52) U.S. Cl. CPC A61K 31/265; A61K 33/34; A61K 33/38; A61P 35/00; C07C 327/40

(57) ABSTRACT

Small, low molecular weight compounds which inhibit the cellular activity of P-type Cu-ATPases, ATP7A and/or ATP7B, and pharmaceutical compositions containing the compounds, are described. Methods of using said compounds in the treatment or prevention of any disease in which copper and/or P1B-type heavy-metal ATPases contribute to disease pathology are also described.

### CROSS REFERENCE TO RELATED APPLICATIONS

This application claims priority under 35 U.S.C. § 119 to provisional patent application U.S. Ser. No. 63/262,611 filed Oct. 15, 2021. The provisional patent application is herein incorporated by reference in its entirety, including without limitation, the specification, claims, and abstract, as well as any figures, tables, appendices, or drawings thereof.

### GOVERNMENT SUPPORT

This invention was made with government support under NIH/NIDDK Grant No. DK116859 and NIH/NCI Grant No. CA262664. The government has certain rights in the invention.

### TECHNICAL FIELD

The present disclosure relates to the field of biotechnology and novel compounds that inhibit the cellular activity of P-type Cu-ATPases, ATP7A and/or ATP7B. More specifically, the present disclosure relates to compositions and methods for modulating the activity of cellular copper transporters and various applications of use thereof.

### BACKGROUND

ATPases play numerous roles in biological processes. P-type ATPases are a superfamily of integral membrane proteins that utilize energy derived from ATP hydrolysis to transport ions across cellular membranes. P-type ATPases are divided into five general subclasses based on the material transported, sequence similarity and overall architecture. Members of the P1B subclass of P-type ATPases are involved in the transport of transition metals and are expressed in bacteria, fungi, plants and mammals. In mammals there are two P1B-type ATPases called ATP7A and ATP7B Both ATP7A and ATP7B reside in the trans-Golgi compartment where they transport copper from the cytoplasm into the lumen of intracellular compartments of the secretory pathway including the trans-Golgi network, melanosomes, and secretory vesicles where copper is incorporated into nascent copper-dependent enzymes Copper-dependent enzymes that receive copper from ATP7A include tyrosinase, which is essential for melanogenesis; the lysyl oxidase family of enzymes, which function in collagen crosslinking in the extracellular matrix; dopamine β-hydroxylase, which converts dopamine to norepinephrine. Copper transported by ATP7B into the secretory pathway is the principal means by ceruloplasmin, a major carrier of copper in blood plasma with roles in iron homeostasis, receives its copper. Additionally, ATP7A and ATP7B are essential for maintaining whole body copper homeostasis via their copper exporting activity. ATP7A is essential for copper entry into the body by exporting dietary copper from intestinal epithelial cells into the blood ATP7A also plays a role in copper entry into the central nervous system ATP7B is essential for removing excess copper from the body via hepatobiliary excretion. Within the cytoplasm, copper is carried to the ATP7A and ATP7B proteins by ATOX1, a small cytosolic copper chaperone which binds to these transporters during copper delivery.

Copper (Cu) is an essential nutrient for all kingdoms of life and serves as a cofactor for enzymes that catalyze a diverse array of biochemical reactions. The redox properties of copper make it essential as an enzymatic cofactor. Nevertheless, excess copper is known to be cytotoxic. Consequently, cells have evolved a highly regulated network of transporters and copper-binding proteins control the distribution and homeostasis of copper. In humans, the importance of copper as an essential nutrient or as a potential cytotoxin is illustrated by genetic diseases caused by mutations in ATP7A or ATP7B. Menkes Disease is a pediatric disorder of copper deficiency caused by mutations in the gene encoding ATP7A. Menkes patients exhibit reduced absorption and tissue distribution of dietary copper, and a deficiency of copper incorporation into to copper-dependent proteins within the body. Menkes patients manifest a range of symptoms reflecting copper deficiency including severe neurological symptoms, connective tissue defects and reduced pigmentation due to loss of ATP7A-dependent copper delivery to the enzyme tyrosinase.

Wilson disease is a genetic disorder of hepatic copper overload caused by a loss of hepatobiliary copper excretion caused by mutations in the gene encoding ATP7B. The elevated hepatic accumulation of copper in Wilson patients primarily manifests as liver disease. Treatments for Wilson disease include the use of copper chelators, penicillamine or trientine, which is taken with food to block the absorption of copper in the intestine. However, such treatments may cause significant neurological deterioration in ˜30% of patients. Thus, identifying alternative treatments for Wilson disease is an important research objective.

Labrador retrievers and other dog breeds are models of Wilson disease due to a missense mutation in the canine homologue of ATP7B that confers a susceptibility to copper-associated hepatitis. Interestingly, a natural variant in the ATP7A gene that reduces ATP7A copper transport function has been identified in certain Labradors as a potential modifier of hepatic copper toxicosis this breed. Such observations provide proof of concept that a small molecule inhibitor that targets intestinal ATP7A may be therapeutic in Wilson disease by limiting dietary copper absorption.

Copper is required in higher amounts by tumor cells relative to normal cells to drive the metabolic demands of cellular proliferation. Copper levels in cancer patients and tumor-bearing animal models are significantly elevated in serum and in tumors and are correlated with poor clinical outcome and response to therapies. Recent studies have demonstrated that copper is an allosteric activator of major oncogenic kinases, including MEK1/2 and ULK1/2. Copper's unique chemistry allows it to be selectively removed from food using highly specific Cu chelators. Oral copper chelators with a clinical history in treating Wilson disease are a promising avenue of cancer treatment. A small molecule inhibitor that targets intestinal ATP7A offers an alternative means of lowering systemic copper levels in cancer patients.

Numerous studies have reported on the role of lysyl oxidases in cancer metastasis, fibrotic disease and scarring. LOX is a secreted copper-dependent amine oxidase that plays critical roles in the development of connective tissue and remodeling of the extracellular matrix by catalyzing the cross-linking of collagen. In addition to LOX, several LOX-like enzymes (LOXL1-4) have been identified that share a conserved catalytic copper-binding domain. To date, functional roles for LOX or LOXL enzymes have been documented in breast, colorectal, prostate, gastric, hepatic, pancreatic, and head and neck cancers, as well as in cancers of the skin, including melanoma. LOX and LOXL enzymes are considered major therapeutic targets for preventing tissue fibrosis and scarring. Topical application of an irreversible small molecule inhibitor of LOX and LOXL enzymes was recently shown to reduce skin scarring and fibrosis. All LOX enzymes require copper for their activity which is supplied by ATP7A during their biosynthesis in the secretory pathway. Targeted disruption of the ATP7A gene in multiple tumor cells is known to block LOX activity and suppress tumor metastasis. A small molecule inhibitor of ATP7A that blocks lysyl oxidase activity has potentially broad applications as a therapy in cancer and fibrotic diseases.

Many studies have implicated a role for ATP7A and ATP7B in the resistance to a range of clinically used chemotherapy drugs. The use of such drugs frequently leads to a loss of therapeutic efficacy due to the selection of drug resistant tumor cells in patients. Increased expression of ATP7A or ATP7B is known to confer resistance to multiple anticancer drugs including cisplatin, vincristine, paclitaxel, etoposide, doxorubicin and mitoxantron. The mechanism by these Cu-ATPases confer drug resistance is not completely understood. The chemodrugs may be directly exported from the cytoplasm by ATP7A/B. Alternatively, depletion of cellular copper levels by upregulation of ATP7A/B may increase the metabolism of these drugs. Nevertheless, targeted inhibition of ATP7A or ATP7B using a small molecule inhibitor offers a novel therapeutic strategy to suppress chemoresistance in cancer cells.

Accordingly, it is an objective of the claimed invention to provide novel compounds and pharmaceutical compositions that inhibit P1B-type copper ATPases, including ATP7A and/or ATP7B.

It is a further objective of the claimed invention to prevent and/or treat a health condition and/or disease in a subject by administering the novel compounds or pharmaceutical compositions.

It is a further objective of the claimed invention to prevent and/or treat chemotherapy resistance in a subject by administering the novel compounds or pharmaceutical compositions.

Other objects, embodiments and advantages of this disclosure will be apparent to one skilled in the art in view of the following disclosure, the drawings, and the appended claims.

### SUMMARY

The present disclosure relates to safe and effective compounds and compositions which modulate the activity of copper transporting P1B-type ATPases from any organism, including ATP7A and/or ATP7B in mammals. The compounds may be used to treat diseases or modulate copper transport and other conditions in a subject in need thereof, including any disease or biological process in which copper and/or P1B-type heavy-metal ATPases contribute.

More specifically, the disclosure provides certain small, low molecular weight compounds which inhibit the cellular activity of P1B-type Cu-ATPases. To identify said low molecular weight compounds, two molecules, MKV1 and MKV3, which share a common core scaffold, were first identified as a potential ATP7A interactor using a computer-based virtual screen. In vitro experiments later demonstrated that MKV1 and MKV3 bind to both ATP7A and ATP7B and inhibit their activities. The most potent of the two molecules, MKV3, was used to develop small, low molecular weight compounds. As disclosed below, these compounds include, but are not limited to: Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, and/or a pharmaceutically acceptable salt or aqueous solution or dispersion thereof. In addition, the compounds include, but are not limited to: Formulae Ia₍₁₋₃₎, Ib₍₁₋₃₎, IC₍₁₋₃₎, IIa₍₁₋₃₎, IIb₍₁₋₃₎, IIc₍₁₋₃₎, IIIa₍₁₋₃₎, IIIb₍₁₋₃₎, IIIc₍₁₋₃₎, IVa₍₁₋₃₎, IVb₍₁₋₃₎, IVc₍₁₋₃₎, Va₍₁₋₃₎, Vb₍₁₋₃₎, Vc₍₁₋₃₎, VIa₍₁₋₃₎, VIb₍₁₋₃₎, VIC₍₁₋₃₎, VIIa₍₁₋₃₎, VIIb₍₁₋₃₎, VIIc₍₁₋₃₎, VIIIa₍₁₋₃₎, VIIIb₍₁₋₃₎, VIIIc₍₁₋₃₎, a resonance structure thereof, and/or a pharmaceutically acceptable salt or aqueous solution or dispersion thereof. These compounds may inhibit P1B-type copper ATPases in different organisms, including human ATP7A and ATP7B.

In some embodiments, the herein disclosed compounds bind to an intramembraneous pocket of P1B-type copper ATPases, including ATP7A and/or ATP7B. In some embodiments, the compounds bind to the entrance of the channel of ATP7A and/or ATP7B, blocking entry of copper into ATP7A and/or ATP7B and disrupting transmembrane copper transport. In some embodiments, disrupting copper delivery via ATP7A or ATP7B inhibits the copper-dependent activity of LOX or LOXL1-4 enzymes. In other embodiments, disrupting copper export via ATP7A or ATP7B increases the sensitivity of cells to exogenous copper.

In some embodiments, competitive inhibition of the P1B-type heavy metal ATPase inhibits the copper-dependent activity of tyrosinase, thereby inhibiting melanogenesis.

In addition, the compounds may be administered to block P1B-type ATPases in microbes, thereby preventing and/or treating microbial infection or microbial resistance to silver and/or copper. In some embodiments, the compounds may be administered to augment the bactericidal or fungicidal properties of silver and copper or to prevent the development of microbial resistance to these metals.

In some embodiments, the compounds may be administered to treat a range of cancers including, but not limited to, carcinoma, blood cancers, sarcoma, mesothelioma, colorectal cancer, pancreatic cancer, head and neck cancer, skin cancer, gastric cancer, breast cancer, prostate cancer, thyroid cancer, endometrial cancer, ovarian cancer, lung cancer, hepatocellular carcinoma, kidney renal papillary cell carcinoma, esophageal cancer, pancreatic ductal adenocarcinoma.

In some embodiments, the compounds may be used to enhance the efficacy or lower the therapeutic dose of anti-cancer chemotherapy drugs, and/or to prevent or reverse resistance to anti-cancer drugs. Such drugs may include cisplatin, vincristine, paclitaxel, SN-38, etoposide, doxorubicin, mitoxantrone, and/or 7-ethyl-10-[4-(1-piperidino)-1-piperidino] carbonyloxycamptothecin (CPT-11).

In some embodiments, the compounds may be administered to treat a disease or condition with a fibrotic component, such as pulmonary fibrosis, hepatic fibrosis, kidney fibrosis, primary sclerosing cholangitis, and/or scarring of the skin or cornea. In some embodiments, the compounds may be administered to treat disorders with an underlying disturbance in copper homeostasis including Menkes disease, Wilson disease, Alzheimer's disease, Amyotrophic lateral sclerosis, Parkinson's disease, Huntington's disease; Creutzfeldt Jakob disease; MEDNIK syndrome. In other embodiments, the compounds may be administered to reduce pigmentation of the skin or to treat pigmentation disorders such as postinflammatory hyperpigmentation, melasma, solar lentigines (sun spots), ephelides (freckles), café au lait macules, vitiligo, pityriasis alba, tinea versicolor, and postinflammatory hypopigmentation.

In some embodiments the effective amount of compound is from about 10 nM to about 1 mM micromolar and the therapy may be administered by oral administration, transdermal administration, topical administration, ocular administration, sublingual administration, parenteral administration, aerosol administration, administration via inhalation, intravenous or intra-arterial administration, local administration via injection or cannula, vaginal administration, and/or rectal administration.

The compounds described herein may be used to modulate copper transport in a subject In some embodiments, the compound competitively inhibits a P1B-type heavy metal ATPase, including a P1B-type copper ATPase such as ATP7A and/or ATP7B. In some embodiments, the compound specifically binds to an intramembraneous pocket of the P1B-type copper ATPase.

While multiple embodiments are disclosed, still other embodiments will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 is a schematic model of the roles of copper (Cu) transporters ATP7A and ATP7B. The CTR1 protein is required for the uptake of Cu across the plasma membrane. ATP7A and ATP7B are located in the trans-Golgi network where they metalate cuproenzymes such as ceruloplasmin (CP) and the lysyl oxidase (LOX) family of proteins. Excess copper concentrations stimulate the trafficking of ATP7A and ATP7B into post-Golgi vesicles that either fuse with the plasma membrane (ATP7A) or lysosomes (ATP7B), a homeostatic mechanism which reduces the accumulation of cytoplasmic copper. ATP7A is ubiquitously expressed, whereas ATP7B is predominantly expressed in hepatocytes.

FIGS. 2A, 2B, and 2C illustrate the role of ATP7A in dietary copper absorption and metalation of secreted copper-dependent enzymes. 2A is a schematic illustration of the roles of CTR1 and ATP7A in dietary copper transport across intestinal enterocytes. Copper import across the apical membrane is facilitated by the CTR1 copper transporter, whereas copper export across the basolateral membrane into the blood is mediated by the ATP7A protein. 2B is a photograph of wild type (WT) and intestine-specific ATP7A knockout mice (ATP7A^{int}) shown 8 weeks after rescue by a single injection of 10 μg/g CuCl₂ at day 7. Note the subtle hypopigmentation in the ATP7A^{int} mice caused by reduced tyrosinase activity. 2C depicts serum ceruloplasmin activity in wild type (WT) and ATP7A^{int} mice at 8 weeks (mean±SEM; ***p<0.001). Low ceruloplasmin activity is a sensitivity biomarker of copper deficiency that is used in clinical studies of copper chelation therapy.

FIGS. 3A, 3B, 3C, and 3D illustrate the role of ATP7A in delivering copper to the LOX family of enzymes. 3A is an illustration of a working model of ATP7A-dependent copper delivery to LOX enzymes (ie, LOX and LOXL1-4). ATP7A pumps copper from the cytoplasm into the Golgi lumen to LOX enzymes as they migrate through the secretory pathway to the extracellular milieu. 3B depicts reduction in secreted LOX activity compared to wild type cells in two independent ATP7A knockout clones of 4T1 breast cancer cells (C3^{7A-} and C8^{7A-}) (mean±SEM; **p<0.01; ***p<0.001). ATP7A knockout was confirmed by immunoblot analysis (inset). 3C depicts restoration of LOX activity in C3^{7A-} cells stably transfected with a plasmid encoding human ATP7A (mean±SEM; ***p<0.001). 3D illustrates how ATP7A is required for the activities of LOX, LOXL1 and LOXL2. Wild type 4T1 and C3^{7A-} cells were transiently transfected with a plasmid encoding GFP alone (control) or in combination with plasmids encoding LOX, LOXL1 or LOXL2. LOX activity was assayed in the media and normalized to cellular GFP expression to control for transfection efficiency (mean±SEM; *p<0.05; ns=not significant; RFU, relative fluorescence units).

FIGS. 4A, 4B, 4C, 4D, and 4E illustrates how ATP7A deletion in 4T1 breast cancer cells attenuates cell migration in vitro, and primary tumor growth and metastasis in vivo. 4A depicts an in vitro scratch assay demonstrating diminished motility of C3^{7A-} cells compared to 4T1 wild type cells. Representative images indicating the extent of gap closure 24 h after scratch formation. Dashed lines indicate scratch starting points. 4B depicts rate of gap closure of scratched 4T1 and C3^{7A-} cell monolayers (mean±SEM; ***p<0.001). 4C depicts weights of primary tumors isolated from mammary glands of BALB/c mice (n=12 per group) 4 weeks after injection with 4T1, C3^{7A-} or C8^{7A-} cells (mean±SEM; ****p<0.0001). 4D are representative images of metastatic nodules (arrowheads) in the lungs of mice bearing 4T1, C3^{7A-} or C8^{7A-} mammary tumors. 4E depicts quantification of metastatic lung nodules per lung (mean±SEM; ****p<0.0001).

FIGS. 5A, 5B, 5C, and 5D are structural models of ATP7A highlighting the pocket targeted for drug design adjacent to the Cu-binding triad of D935, E798 and M746 amino acids. 5A is a model of the ATP7A protein (backbone view) showing the platform (ribbon helix) at the cytosolic-membrane interface and the conserved triad of closely arranged Met (M), Glu (E), Asp (D) residues at the mouth of the funnel. 5B is a close-up of the platform region showing copper (Cu) bound within the triad in a predicted trigonal planar configuration. 5C is a space-filling model of ATP7A highlighting a pocket adjacent to the triad that was targeted for drug design. 5D depicts MKV3 docked within the pocket.

FIGS. 6A, 6B, 6C, and 6D characterize MKV3 and MKV1 binding to ATP7A/B. 6A depicts the chemical structure of MKV1. 6B depicts the chemical structure of MKV3. 6C is a model of MKV3 docked within the binding pocket of ATP7A. Coordinating residues D935 and E798 are predicted to bind copper during transport. 6D is a graph showing the analysis of MKV3 and MKV1 binding to ATP7A and ATP7B using microscale thermophoresis (MST). Detergent-free HEK293 cell lysates containing GFP-ATP7A, GFP-ATP7B or GFP alone as a negative control were subjected to MST following the addition of MKV3 or MKV1. 6E is a table of the calculated affinities of MKV3 and MKV1 for both ATP7A and ATP7B.

FIGS. 7A, 7B, and 7C demonstrate that a glutamate at position 1030 (E1030) of human ATP7A within the predicted binding pocket is a determinant of MKV3 binding affinity. 7A is a model of MKV3-coordinating residues in ATP7A which are identical to those of ATP7B except that the negatively charged glutamate at position 1030 (E1030) of ATP7A corresponds to a positively lysine at position 1013 (K1013) in ATP7B. The residues E1030 of ATP7A and K1013 of ATP7B are predicted to interact with a negatively charged fluorine atom in MKV3. The electronegative fluorine is predicted to bind more strongly to lysine relative to glutamate and may explain, in part, the higher affinity of MKV3 for ATP7B. To confirm this the E1030 residue in ATP7A was mutated to a lysine to generate an ATP7A(E1030K) mutant protein. 7B is a graph showing microscale thermophoresis analysis of MKV3 binding to ATP7A(E1030K) as well as wild type ATP7A and ATP7B. Detergent-free HEK293 cell lysates containing GFP-ATP7A, GFP-ATP7A(E1030K), GFP-ATP7B or GFP alone as a negative control were subjected to MST following the addition of MKV3. 7C is a table of the calculated binding affinities of MKV3 for ATP7A(WT), ATP7A (E1030K) and ATP7B. The results show that MKV3 has a higher affinity for ATP7A(E1030K) relative to wild type ATP7A, thus providing experimental support that MKV3 interacts with ATP7A via E1030 and with ATP7B via K1013, and confirms the in silico modeling of MKV3 in the binding pocket of ATP7A/B.

FIGS. 8A, 8B and 8C demonstrate that MKV3 is specific for ATP7A and ATP7B. 8A illustrates a copper sensitive fibroblast cell line (Cu^{S}) generated by deleting genes encoding ATP7A, metallothionein-I (MT-I) and metallothionein-II (MT-II). Cu^{S} cells cannot be propagated in regular medium (DMEM) unless a copper chelator (BCS) is supplied to the medium. 8B shows that Cu^{S} cells can be rescued (complemented) in regular medium by stable transfection with plasmids encoding ATP7A, ATP7B or MT-II, but not by vector control. 8C demonstrates that MKV3 enhances copper toxicity in the Cu^{S} cells complemented with ATP7A or ATP7B, but not in the Cu^{S} cells complemented with MT-II. 8C is a graph depicting the survival of Cu^{S} cells stably transfected with plasmids encoding ATP7A, ATP7B or MT-II and grown in the presence (+) or absence (−) of MKV3 (10 μM) or in the presence or absence of the 50% cytotoxic copper concentration for each stably transfected Cu^{S} cell line. Notably, MKV3 enhanced copper toxicity in the Cu^{S} cells complemented with ATP7A or ATP7B, but not in the Cu^{S} cells complemented with MT-II. This indicates that MKV3 sensitizes cells to copper in a manner that is dependent on the presence of ATP7A or ATP7B, consistent with MKV3-mediated inhibition of these transporters.

FIGS. 9A, 9B, and 9C depict MKV1 and MKV3 inhibition of ATP7A activity in vitro and in vivo. Previous studies have shown that ATP7A is essential for copper delivery into the secretory pathway to tyrosinase, a copper-dependent enzyme required for melanogenesis. If MKV1 and MKV3 are inhibitors of ATP7A function, they should block tyrosinase activity in melanoma cells. 9A shows bright field microscopy of in situ tyrosinase activity within B16 melanoma cells (ATP7A WT) visualized by the colorimetric conversion of L-DOPA to DOPAchrome. To confirm that ATP7A is required for tyrosinase activity, B16 melanoma cells were generated in which the ATP7A gene was deleted by CRISPR-CAS9 to generate ATP7A^{KO} cells. As expected, tyrosinase activity was absent in the B16 ATP7A^{KO} cells. Treatment of wild type B16 melanoma cells with MKV1 (50 μM) or MKV3 (5 μM) for 24 h resulted in a loss of tyrosinase activity. 9B is a graph depicting quantitative analysis of tyrosinase activity in wild type B16 cells treated for 24 h with a range of concentrations of MKV1 or MKV3. The 50% inhibitory concentration (IC50) of MKV1 and MKV3 for tyrosinase activity is shown. 9C depicts MKV3 inhibition of melanin production in melanoma tumors on the lungs of mice. Wild type B16 melanoma cells were injected by tail vein into C57BL/6 mice to establish metastatic tumors on the lungs. These tumor-bearing mice were subcutaneously injected with either MKV3 (50 mg/kg) or vehicle alone (1% DMSO) every other day for 7 days. Left panel images show the lungs of three different mice immediately after dissection. In control mice, darkly pigmented melanoma nodules were readily apparent on the lungs (white arrows). By contrast, the nodules on the lungs of MKV3-treated mice had very little pigment (white arrows). Staining with Bouin's solution (middle panels) revealed a striking loss of pigmentation in melanoma nodules in the lungs of MKV3-treated mice compared to the darkly pigmented nodules in control mice (black arrows). Taken together, these data indicate that MKV3 blocks ATP7A-dependent tyrosinase activity in vitro and in vivo.

FIGS. 10A, 10B, 10C, 10D, 10E, 10F and 10G demonstrate that MKV3 inhibits the activation of tyrosinase activity by ATP7B. Prior studies have shown that exogenously expressed ATP7B can activate tyrosinase in cells lacking ATP7A. For these studies, a B16 melanoma cell model was used in which tyrosinase activity can be detected by in situ oxidation of L-DOPA to DOPAchrome (10A). As shown earlier, B16 cells deleted for the ATP7A gene (ATP7A^{KO}) completely lack tyrosinase activity (10B). The ATP7A^{KO} cells were transiently transfected with plasmids expressing either human ATP7A (hATP7A) or ATP7B (hATP7B) and then the cells were incubated for 24 h with or without MKV3 added to the media (10 μM). In situ tyrosinase assays revealed that both ATP7A and ATP7B restored tyrosinase activity in ATP7A^{KO} melanoma cells (10C and 10E) and that MKV3 treatment prevented activation of tyrosinase activity by ATP7A and ATP7B (10D and 10F). Quantification of tyrosinase activity in these cells is shown (10G). Data represent mean±SEM; ****p<0.0001 of three independent replicates. These data indicate that MKV3 blocks both ATP7A and ATP7B-dependent copper transport activity.

FIGS. 11A, 11B and 11C depict MKV1 and MKV3 inhibition of lysyl oxidase activity. ATP7A is required to deliver copper to LOX enzymes in the secretory pathway, thus a study was performed to test whether LOX activity secreted into the medium is reduced in cells treated with MKV1 or MKV3. FIGS. 11A and 11B show that MKV3 causes a reduction in LOX activity in the conditioned media of LLC lung cancer cells (11A) and 4T1 breast cancer cells (11B). Cells were treated for 4 days with 5 μM MKV3 or 1% DMSO (vehicle) (mean±SEM; ****p<0.0001; RFU, relative fluorescence units). FIG. 11C shows the 50% inhibitory concentrations (IC50) of MKV1 and MKV3 for LOX activity in 4T1 breast cancer cells.

FIGS. 12A, 12B, 12C and 12D depicts the inhibitory effects of MKV3 on the motility of both 4T1 breast cancer cells and LLC lung cancer cells using in an in vitro scratch assay. LOX activity is known to be important for the motility of cancer cells. In this assay, a gap (scratch) was created in a confluent monolayer of cells and closure of the gap in the presence or absence of MKV3 was monitored by video microscopy. Gap closure in 4T1 (12A) and LLC (12B) cells was completed within 24 h in media containing 1% DMSO (vehicle control), whereas the rate of gap closure was reduced in cells treated with MKV3 (10 μM). Dashed lines indicate scratch starting points (12A and 12C). Compared to vehicle control, MKV3 treatment significantly reduced the rates of gap closure for both 4T1 (12B) and LLC cells (12D) (mean±SEM; ***p<0.001; ****p<0.0001). The requirement for ATP7A in cancer cell motility is consistent with its role in activation of lysyl oxidase enzymes.

FIGS. 13A, 13B and 13C are immunofluorescence microscopy images depicting the inhibitory effects of MKV3 on copper-stimulated trafficking of ATP7A. Since the copper-stimulated trafficking of ATP7A is dependent on its transport activity, an inhibitor of ATP7A would be expected to block copper-stimulated trafficking of ATP7A. 4T1 breast cancer cells were pre-grown on glass coverslips for 16 hours in media containing 10 μM of the copper chelator bathocuproine disulfonate to create a baseline of low copper levels. The cells were rinsed in PBS and then transferred to media containing 1% DMSO (vehicle control) (13A), 1% DMSO plus 2 μM copper chloride (13B), or 1% DMSO plus 10 μM MKV3 (13C). After 4 h, the cells were fixed, permeabilized and stained with anti-ATP7A antibodies (green). Nuclei were labeled with DAPI stain. In vehicle treated control cells, the ATP7A protein was strongly detected in the perinuclear region (13A), which is consistent with its known location in the trans-Golgi compartment. As expected, the copper treatment stimulated ATP7A trafficking from the perinuclear region into cytoplasmic vesicles (13B). The copper-stimulated trafficking of ATP7A was blocked by MKV3 (13C). These results provide further evidence that MKV3 is an inhibitor of ATP7A activity.

FIGS. 14A, 14B and 14C show the effect of MKV3 on total copper levels in three different cell lines, 4T1 (14A), B16 (14B), and HEK293 (14C). Since ATP7A functions in copper export from cells, an inhibitor of ATP7A activity would be expected to increase cellular copper concentrations. 4T1, B16 and HEK293 cell lines were incubated in media containing MKV3 (10 μM) or vehicle alone (1% DMSO). After 24 hours, cellular copper concentrations were measured using inductively coupled plasma mass spectroscopy (ICP-MS). MKV3 treatment resulted in significantly increased concentrations of copper in all three cell lines (mean±SEM; **p<0.01; ***p<0.001; ****p<0.0001). These results provide further evidence that MKV3 is an inhibitor of ATP7A activity.

FIGS. 15A, 15B, 15C, and 15D provide evidence that MKV3 increases the sensitivity of cells to copper. It is well-known that copper export via ATP7A is the major mechanism by which cells are protected against rising copper levels, which is facilitated by Cu-stimulated trafficking of ATP7A from the trans-Golgi to the plasma membrane. Therefore, a predicted consequence of an ATP7A inhibitor would be an increase of the sensitivity of cells to high copper concentrations. The sensitivity of 4T1, B16 and LLC cells to a range of elevated copper concentrations over a 48 h period in media containing 1% DMSO vehicle (−MKV3) or the same media containing 10 μM MKV3 (+MKV3) was tested. As shown in 15A, 15B and 15C, the presence of MKV3 strongly augmented copper toxicity in all three cell lines. 15D tabulates the 50% cytotoxic copper concentration (CC₅₀) calculated for each cell line in the presence or absence of MKV3. These data provide further support that MKV3 inhibits ATP7A copper transport activity.

FIG. 16 demonstrates that MKV3 increases the sensitivity of cells to copper and silver, but not other heavy metals. Across all kingdoms of life, copper transporting P1B-type ATPases have been shown to transport silver (Ag⁺) due to its electrical similarity to Cu¹⁺. Therefore, it was tested whether MKV3 increases the sensitivity of cells to silver relative to other metals using the crystal violet assay. Survival of 4T1 cells was determined after exposure to a range of metal ion concentrations in media containing 1% DMSO vehicle (-MKV3) or the same media containing 10 μM MKV3 (+MKV3). All metals were added as chloride salts except silver, which was added as silver nitrate (AgNO₃). The data, which are expressed as percent survival relative to no added metal, demonstrate that MKV3 conferred sensitivity to as little as 5 μM copper and silver (ie, known substrates of ATP7A), whereas MKV3 did not affect the sensitivity to other metals up to 50 μM. These findings are consistent with MKV3 being an inhibitor of ATP7A/B

FIGS. 17A, 17B, 17C, and 17D demonstrate that MKV3 given intravenously or subcutaneously reduces primary tumor growth and metastasis of 4T1 mammary cancer cells in syngeneic BALB/c mice. In these experiments, 4T1 cells stably expressing mCherry were used to allow quantitative PCR analysis of metastatic burden in the lungs. 17A and 17B demonstrate that the weights of 4T1 primary tumors in the mammary glands as well as the metastatic lung burden were reduced in mice given intravenous treatments of MKV3 (50 mg/kg) compared to vehicle control (5% DMSO and 5% TW-80). The mice were injected via tail vein with either MKV3 (50 mg/kg) or vehicle every Tuesday and Thursday for 3 weeks. Data are presented as the mean±SEM; **p<0.01. 17C and 17D demonstrate that MKV3 given subcutaneously to tumor bearing BALB/c mice significantly reduced primary tumor growth and lung metastasis compared to vehicle controls. MKV3 (50 mg/kg) or vehicle (5% DMSO and 5% TW-80) was administered subcutaneously every Monday, Wednesday and Friday for 3 weeks. Data are presented as the mean±SEM; **p<0.01; ****p<0.0001.

FIGS. 18A, 18B and 18C demonstrate that MKV3 increases the accumulation and cytotoxicity of doxorubicin (DOX). Previous studies have shown that elevated expression of ATP7A or ATP7B in cells confers increased tolerance to a wide array of cancer chemotherapy drugs including DOX, a molecule whose auto-fluorescent properties allow it to be quantified in cultured cells. 18A demonstrates that the addition of MKV3 (10 μM) to the culture medium of 4T1 breast cancer cells significantly enhanced the cytotoxicity of DOX (0.6 μM) compared to 1% DMSO vehicle control (mean±SEM; ****p<0.0001). 18B demonstrates that MKV3 significantly increased the time-dependent accumulation of DOX in the nucleus of 4T1 cells compared to 1% DMSO vehicle control. 18C provides a quantitative analysis of DOX fluorescence in 4T1 cells treated with MKV3 versus compared to 1% DMSO vehicle. These results demonstrate that MKV3 increases the accumulation and toxicity of DOX and are consistent with effects of ATP7A deletion in previous studies.

FIGS. 19A, 19B, and 19C demonstrate that MKV3 inhibits bacterial P1B-type ATPases. Methicillin-resistant Staphylococcus aureus (MRSA) is a human pathogen that expresses two Cu-transporting P1B-type ATPases, CopA and CopB, which are known to enhance the virulence of this bacterium. 19A illustrates CopA and CopB copper exporters at the plasma membrane of S. aureus. Like all Cu-transporting P1B-type ATPases, both CopA and CopB possess the conserved copper binding triad that overlaps a predicted MKV3 binding pocket. To test whether MKV3 inhibits bacterial Cu-ATPases it was tested whether MKV3 can sensitize S. aureus to copper. 19B demonstrates that the growth of wild type S. aureus was not affected by either MKV3 (0.5 μM in 1% DMSO vehicle) or copper (50 μM) when these were added separately. However, when added in combination both MKV3 (0.5 μM) and copper (50 μM) produced significant inhibition of growth which could be prevented by addition of a copper chelator BCS (mean±SEM; ns: not significant, **p<0.01). 19C examines the effect of MKV3 and copper on the growth of a copper-sensitive mutant strain of S. aureus in which both CopA and CopB genes were deleted (ΔcopA/ΔcopB). The growth of the ΔcopA/ΔcopB strain was inhibited by approximately 50% when copper alone (50 μM) was added to the culture medium. However, unlike the wild type strain, MKV3 did not increase copper sensitivity in the ΔcopA/ΔcopB strain (mean±SEM; ns: not significant). These data indicate that the ability of MKV3 to increase copper sensitivity in S. aureus is dependent on CopA/B expression. 19D demonstrates that MKV3 also increases the sensitivity of wild type S. aureus to silver, a known substrate of copper transporting P-type ATPases (mean±SEM; ns: not significant, ****p<0.0001).

FIGS. 20A, 20B, 20C, and 20D demonstrate the importance of the sulfur in MKV3 for copper binding and inhibition of ATP7A. The predicted MKV3 binding pocket in copper transporting P1B-type ATPases is located in close proximity to the copper-binding triad at the transporter entrance. The thioamide in MKV3 led to speculation that Cu(I) may bind to the sulfur atom in MKV3, thus preventing passage into the membrane spanning channel domain. To test this hypothesis, a control compound was generated in which the sulfur atom in the thioamide was replaced with oxygen to generate a corresponding amide (MKV3-D1). 20A and 20B show the chemical structures of MKV3 and MKV3-D1 as well as the results of NMR experiments using MKV3 and MKV3-D1 in the presence and the absence of CuI in 9:1 d6-DMSO/D2O. The results show that MKV3 signals change upon addition of CuI while no such changes were observed with MKV3-D1. To test whether the thioamide in MKV3 is essential for inhibition of ATP7A copper transport activity, the potency of MKV3 and MKV3-D1 in blocking tyrosinase activity in B16 melanoma cells was compared. 20C is a graph of the relationship between tyrosinase activity and compound concentration. The data show that MKV3-D1 has a far higher tyrosinase 50% inhibitory (IC₅₀) concentration than MKV3. 20D is a graph of the relationship between the survival of 4T1 breast cancer cells and compound concentration in the presence or absence of 10 μM copper. The data show that 10 μM copper resulted in a 47-fold reduction in the MKV3 concentration required to kill 50% of 4T1 cells (CC₅₀) (63.13 μM vs 1.34 μM). By contrast, 10 μM copper reduced the CC₅₀ of MKV3-D1 by only 5.6 fold (106.50 vs 19.06). These data indicate that the thioamide in MKV3 is required for both copper binding and inhibitory activity.

Various embodiments of the present disclosure will be described in detail with reference to the drawings, wherein like reference numerals represent like parts throughout the several views. Reference to various embodiments does not limit the scope of the disclosure. Figures represented herein are not limitations to the various embodiments according to the disclosure and are presented for exemplary illustration of the invention. An artisan of ordinary skill in the art need not view, within isolated figure(s), the near infinite number of distinct permutations of features described in the following detailed description to facilitate an understanding of the present invention.

### DETAILED DESCRIPTION

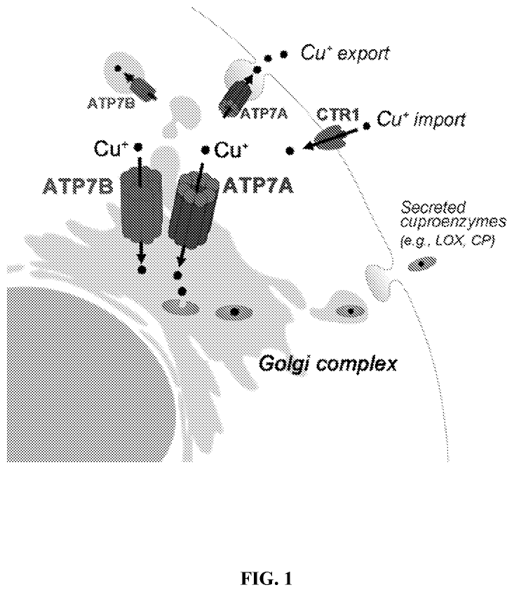

ATP7A and ATP7B are P-type ATPases that transport copper across cell membranes. Genetic disruption of ATP7A is known to perturb copper metabolism, suppress tumor growth and metastasis, potentiate cisplatin chemotherapy, and inhibit melanin production. A small molecule inhibitor of ATP7A and/or ATP7B has potential biomedical applications in cancer, infectious disease, fibrotic scarring, Menkes disease, Wilson disease and other diseases or conditions in which copper contributes to the pathology (See FIG. 1). A molecular scaffold called MKV3 was identified as a potential ATP7A interactor using a computer-based virtual screen. In vitro and in vivo experiments demonstrate that MKV3 binds to ATP7A and inhibits its activity. Notably, the ability of MKV3 or its derivatives to inhibit any copper transporter has not been previously reported.

Generally, P1B-type heavy-metal ATPases are integral membrane proteins that play a key role in metal homeostasis by selectively moving heavy metals through membranes. P1B-type ATPases are highly conserved across all life forms including bacteria, plants, and animals. The ion pumps in the P-type ATPase superfamily share a common enzymatic mechanism in which ATP hydrolysis aids in transporting ions across the membrane.

P-type ATPases are a large superfamily of integral membrane proteins found in all types of living organisms that translocate a diverse set of substrates including hard metals (e.g., H⁺, Na⁺, K⁺, and Ca²⁺), soft metals (e.g., heavy metals such as Cu⁺, Zn²⁺, Cd²⁺), and possibly lipids. This superfamily is divided into five major branches and ten subfamilies, that differ according to the substrate being transported. All the heavy-metal pumps from bacteria, plants, and humans share significant sequence similarities and are clustered together as the P1B subfamily. P1B-type heavy-metal ATPases (HMAs) have been implicated in the transport of a range of essential as well as potentially toxic metals across cell membranes. The present disclosure contemplates inhibition of P1B-type heavy-metal ATPases in a wide range of subjects, including bacteria, plants, and humans.

HMAs directly transport copper into the body, out of the body and into metalloenzymes such as lysyl oxidase and tyrosinase. The herein disclosed HMA inhibitor compounds are contemplated to prevent or treat a wide range of diseases and conditions, including any disease in which copper or HMAs contribute to the disease pathology and any disease in which blocking copper transport is beneficial. In other embodiments, the HMA inhibitors treat or prevent metabolic diseases of copper such as Menkes disease or Wilson disease. In other embodiments, the HMA inhibitors treat or prevent cancers or augment the therapeutic efficacy of cancer treatments. In other embodiments, the HMA inhibitors treat or prevent fibrotic pathology in any tissue contributed by the activity of lysyl oxidases (LOX or LOXL1-4) such as scarring of the skin or cornea, or other fibrotic diseases such as scleroderma, rheumatoid arthritis, Crohn's disease, ulcerative colitis, myelofibrosis and systemic lupus erythematosus. Prior studies have shown that genetic deletion of ATP7A reduces angiogenesis. Thus, in another embodiment, the HMA inhibitors treat or prevent diseases associated with excess angiogenesis such as ischemic cardiovascular diseases or eye related neovascular diseases such as proliferative diabetic retinopathy, age-related macular degeneration, and retinal vein occlusion. Prior studies have shown that genetic mutations in ATP7A or ATP7B improves the pathology of mouse models of amyotrophic lateral sclerosis and Alzheimer's disease. Thus, in another embodiment, HMA inhibitors are expected to treat or prevent neurological diseases in which copper or HMAs contribute to disease pathology including amyotrophic lateral sclerosis and Alzheimer's disease.

In another embodiment, the claimed compounds inhibit growth of methicillin-resistant Staphylococcus (MRSA). Methicillin-resistant Staphylococcus aureus infection is caused by a type of Staphylococcus bacteria that has become resistant to many of the antibiotics used to treat ordinary Staphylococcus infections. MRSA infection is one of the leading causes of hospital-acquired infections and is commonly associated with significant morbidity, mortality, increased length of hospital stay, and cost burden.

In another embodiment, the claimed compounds increase the accumulation of the anti-cancer drug doxorubicin and increase the sensitivity of breast cancer cells to this drug. Recent studies have implicated ATP7A and ATP7B in the resistance to the anti-cancer drug, cisplatin (cis-diamminedichloroplatinum or DDP), which has been used in chemotherapy for various cancerous tumors, and particularly in the treatment of testicular and ovarian cancers. Cisplatin reacts with nuclear DNA and prevents normal replication which affects rapidly dividing cancer cells. Eventually, as is the case with most anticancer drugs, patients develop drug resistant cells which do not respond to this therapy. It was also reported that a higher level of ATP7B expression is often associated with tumor resistance to cisplatin. Silencing ATP7B expression was associated with decreased cell survival in the presence of cisplatin while an increase in ATP7A expression correlated with increased tumor resistance to cisplatin. ATP7A expression is also required for resistance to other chemotherapy drugs such as vincristine, paclitaxel, SN-38, etoposide, doxorubicin, mitoxantron, and 7-ethyl-10-[4-(1-piperidino)-1-piperidino]carbonyloxycamptothecin (CPT-11). Thus, in another embodiment, decreasing anti-cancer drug resistance including chemotherapy resistance (e.g., cisplatin and doxorubicin resistance) via administration of the herein described compounds is contemplated. Indeed, administration of the present compounds to a subject may lower the effective therapeutic doses of doxorubicin, cisplatin and similar anti-cancer drugs.

Notably, inhibiting the activity of the copper-transporters and other metal transporters may have implications in various treatments and therapies for ATP7A and/or ATP7B-related conditions. Reflecting copper's properties as both essential and potentially toxic, cancer cells are known to be susceptible to both copper depletion and copper excess. Drugs that lower copper concentrations (chelators) or elevate copper concentrations (ionophores) have been used in both pre-clinical and clinical cancer studies. The present compounds may be administered to block intestinal entry of copper or to enhance copper levels systemically to treat various cancers including, but not limited, to blood cancers such as leukemia, lymphoma, myeloproliferative disorder, myelodysplatic syndromes, multiple myeloma as well as solid cancers such as carcinoma, sarcoma, mesothelioma, colorectal cancer, pancreatic cancer, head and neck cancer, esophageal cancer, skin cancer, gastric cancer, breast cancer, prostate cancer, thyroid cancer, endometrial cancer, ovarian cancer, lung cancer, hepatocellular carcinoma, and/or kidney renal papillary cell carcinoma. The present compounds may be administered in combination with other therapies, or treatments that increase or decrease bioavailable copper.

In other embodiments, the present compounds may be administered to treat or prevent diseases of fibrosis in which lysyl oxidase enzymes, which require ATP7A for their activity, are known to play a role. These include pulmonary fibrosis, hepatic fibrosis, heart fibrosis, skin fibrosis, scleroderma or systemic sclerosis and/or primary sclerosing cholangitis. In other embodiments, the present compounds may be administered to block angiogenesis in neovascular diseases such as diabetic retinopathy, treat or prevent disorders of copper metabolism such as Wilson disease, to treat or prevent neurological conditions that have an underlying disruption in copper homeostasis such as Alzheimer's disease, or to reduce pigmentation and scarring of the skin.

In other embodiments, the present compounds may be used to inhibit copper transporting P-type ATPases that regulate biological processes across an array of organisms. For example, in crop plants the present compounds may be used to control fruit ripening or other processes related to the ethylene receptor which acquires copper from a P1B-type ATPase. In other embodiments, the present compounds may be used to control infections by the growing list of pathogenic organisms whose virulence is known to depend on one or more functional copper transporting P1B-type ATPases including Staphylococcus aureus, Mycobacterium tuberculosis, Acinetobacter baumannii, Salmonella typhimurium, Pseudomonas aeruginosa, Listeria Monocytogenes, Streptococcus pneumoniae, Leishmania major, Plasmodium berghei and Botrytis cinerea. Examples include the control of Staphylococcus aureus (MRSA) infections as a topical treatment to augment the antibiotic properties of silver and copper used in wound care.

### Definitions and Interpretation

So that the present disclosure may be more readily understood, certain terms are first defined. Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. The nomenclature used in connection with, and the laboratory procedures and techniques of, analytical chemistry, synthetic organic chemistry, and medicinal and pharmaceutical chemistry described herein are those well-known and commonly used in the art.

It is to be understood that all terminology used herein is for the purpose of describing particular embodiments only and is not intended to be limiting in any manner or scope. For example, as used in this specification and the appended claims, the singular forms “a,” “an” and “the” are used interchangeably and can include plural referents unless the content clearly indicates otherwise. Also, as used herein, “and/or” refers to and encompasses any and all possible combinations of one or more of the listed items, as well as the lack of combinations when interpreted in the alternative (“or”). Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

As used herein, the term “about,” refers to variation in the numerical quantity that can occur, for example, through typical measuring techniques and equipment, with respect to any quantifiable variable, including, but not limited to, mass, amount of substance, volume, time, length, diameter, percent, quantity, and concentration. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The terms “administration of” or “administering” an active agent should be understood to mean providing an active agent to the subject in need of treatment in a form that can be introduced into that individual's body in a therapeutically useful form and therapeutically effective amount.

The term “analog” as used herein refers to a molecular derivative of a molecule. The term is synonymous with “structural analog” or “chemical analog.”

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, “competitive inhibition” refers to the interruption of a chemical pathway owing to one chemical substance inhibiting the effect of another by competing with it for binding or bonding. Any metabolic or chemical messenger system can potentially be affected by this principle, but several classes of competitive inhibition are especially important in biochemistry and medicine, including the competitive form of enzyme inhibition, the competitive form of receptor antagonism, the competitive form of antimetabolite activity, and the competitive form of poisoning. Relatedly, a receptor antagonist is a type of receptor ligand or drug that blocks or dampens a biological response by binding to and blocking a receptor rather than activating it like an agonist. Antagonist drugs interfere in the natural operation of receptor proteins and are sometimes referred to as “blockers”. In competitive inhibition of enzyme catalysis, binding of an inhibitor prevents binding of the target molecule of the enzyme, also known as the substrate. This is accomplished by blocking the binding site of the substrate (e.g., the “active site”) by some means.

As used herein, “condition” or “health condition” refers to an ex vivo, in vivo, or in cellulo state of a subject or organism. A health condition may relate to, for example, the presence of health-related microorganisms in a given location. In the present context, heavy metal transporters are ubiquitous in nature, and therefore the claims contemplate modulation of metal ion transport in a wide variety of contexts including application to the in vivo and ex vivo killing or modulation of bacteria, fungus, and viruses. The range of subject animal species that may suffer from a health condition is also very broad, including humans, domesticated animals, farm animals, and aquatic invertebrates. Notably, health conditions can include diseases, infections, and any metal ion homeostatic condition.

As used herein, “disease” refers to a condition of a living animal or plant body or of one of its parts that impairs normal functioning and is typically manifested by distinguishing signs and symptoms. Diseases may include bacterial infections, viral infections, resistant viral and bacterial infections, genetic disorders, cancers, any conditions that involve a copper homeostatic component, and other harmful health conditions known in the art.

As used herein, “resonance” refers to the bonding in a molecule or compound where there can be variations or combinations of several contributing structures in a resonance hybrid according to valence bonding theory. As referred to herein, resonance structures can have delocalized electrons in some molecules where bonding is not expressed by a single structure, such that contributing structures can differ by the position of delocalized electrons.

As used herein, “transporter” or “heavy metal transporter” refers to any protein or enzyme that transports metal ions. This definition also encompasses P1B-type copper ATPases such as ATP7A and/or ATP7B.

As used herein, “therapeutically effective amount” refers to the amount of a compound or pharmaceutical composition administered to improve, inhibit, or ameliorate a condition of a subject, or a symptom of a disorder, in a clinically relevant manner. Any improvement in the subject is considered sufficient to achieve treatment. In some embodiments, a therapeutically effective amount is an amount that prevents or reduces the likelihood of tumor metastasis in a statistically significant way. In some embodiments, a therapeutically effective amount is an amount that prevents or reduces the occurrence or one or more symptoms of a pathology, or is an amount that reduces the severity of, or the length of time during which a subject suffers from, one or more symptoms of the pathology (for example, by at least 10%, 20%, 30%, 40%, 50%, 60%, 70%, 80%, 90%, 95%, 99%, or more, relative to a control subject that is not treated with the compound or pharmaceutical composition). Moreover, the “therapeutically effective amount” will vary depending on the particular compound or pharmaceutical composition administered, on the severity of the condition being treated, individual patient parameters including age, physical condition, size and weight, concurrent treatment, frequency of treatment, and the mode of administration. These factors are well known to those of ordinary skill in the art and can be addressed with no more than routine experimentation.

As used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt.

As used herein, “treating” or “treatment” of a state, disorder or condition includes: (1) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statically significant or at least perceptible to the patient or to the physician.

As used herein, “subject” refers to the living organism to which the herein disclosed compounds are administered. In the present context, potential subjects are ubiquitous in nature, including any living organism, including plants, fungi, protists, bacteria, and animals. The range of animal species that may comprise subjects is very broad and includes, for example, humans, domesticated animals, farm animals, aquatic vertebrates, and aquatic invertebrates.

### Compounds

Compounds that competitively inhibit a P1B-type heavy metal ATPase include the following formulae:

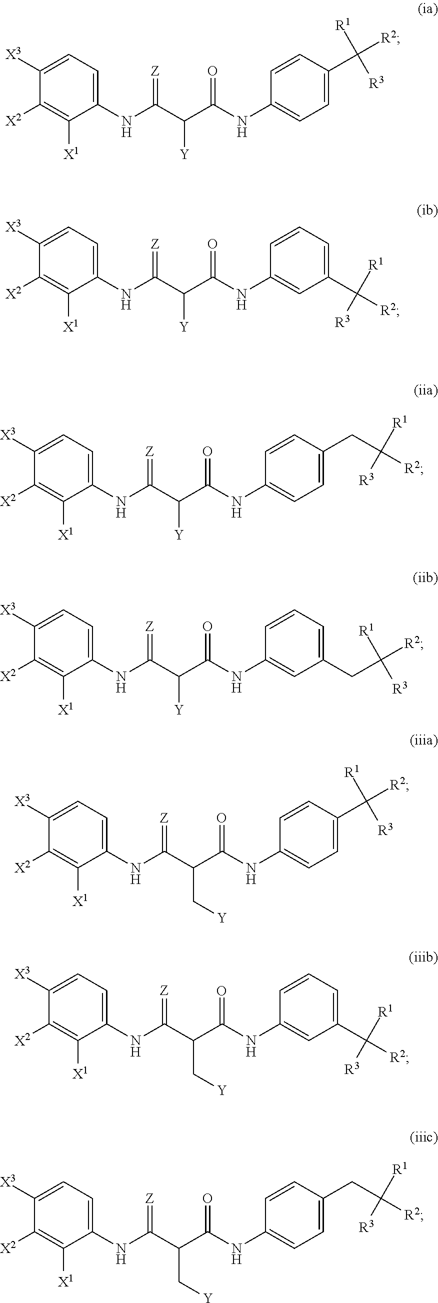

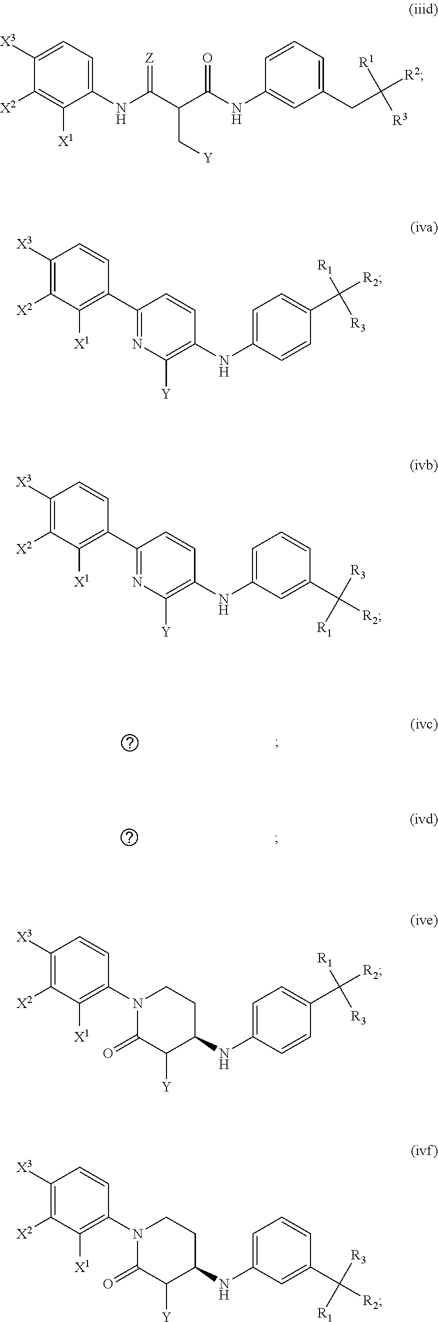

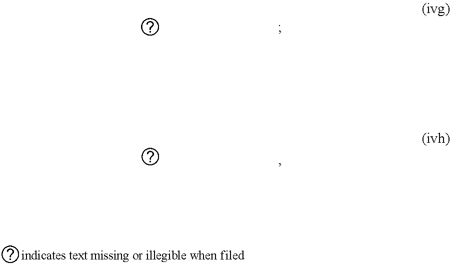

a resonance structure thereof, or a pharmaceutically acceptable salt or aqueous solution or dispersion thereof,
wherein each of R¹, R², and R³ individually are a halogen, hydrogen, hydroxyl, amine (NH₂),

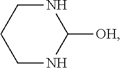

or absent;
wherein X¹, X², and X³ individually are a hydrogen, halogen, O, or C═O;
wherein at least two of X¹, X², and X³ are hydrogen;
wherein Y is HC≡N, CH₃, CH₃—CH₄, C═O, hydrogen, absent; and
wherein Z is S, O, or amine (NH₂).

Compounds that competitively inhibit a P1B-type heavy metal ATPase preferably include the following formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc:

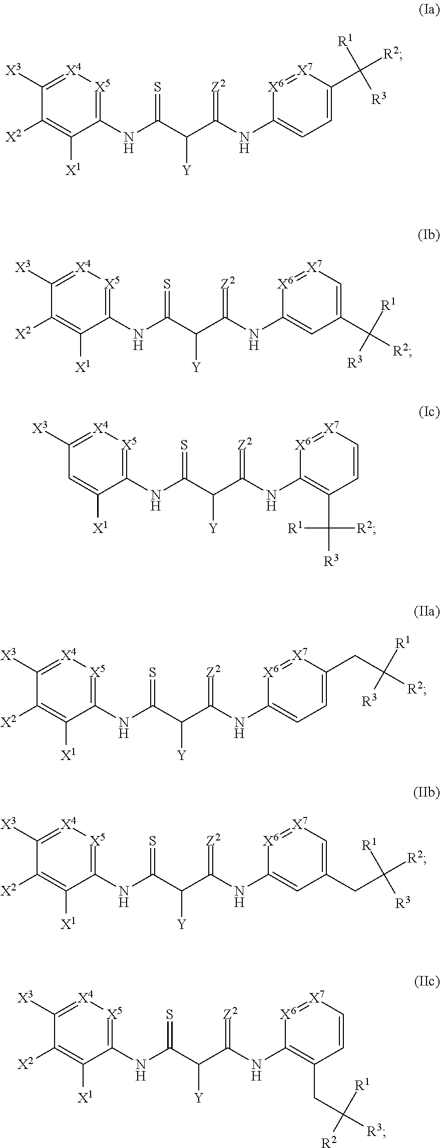

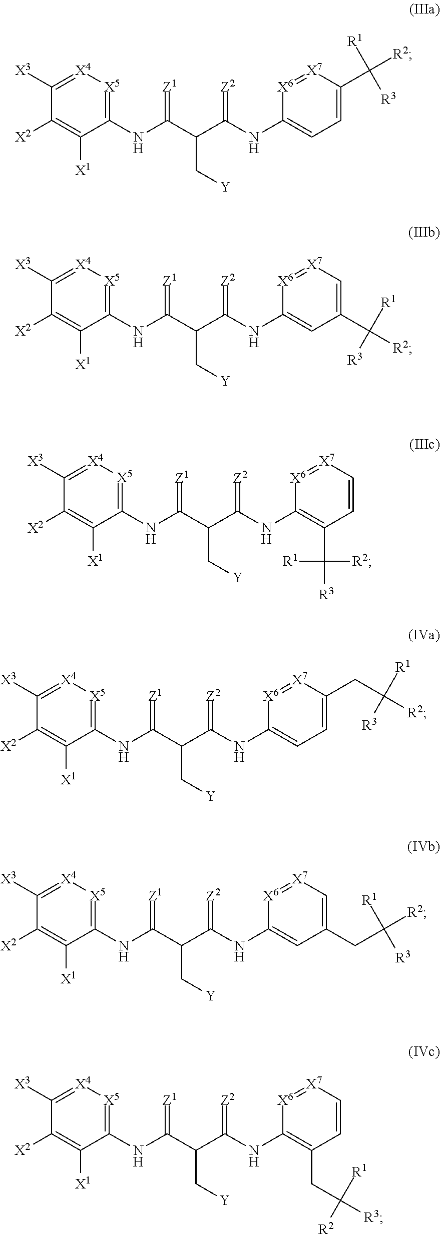

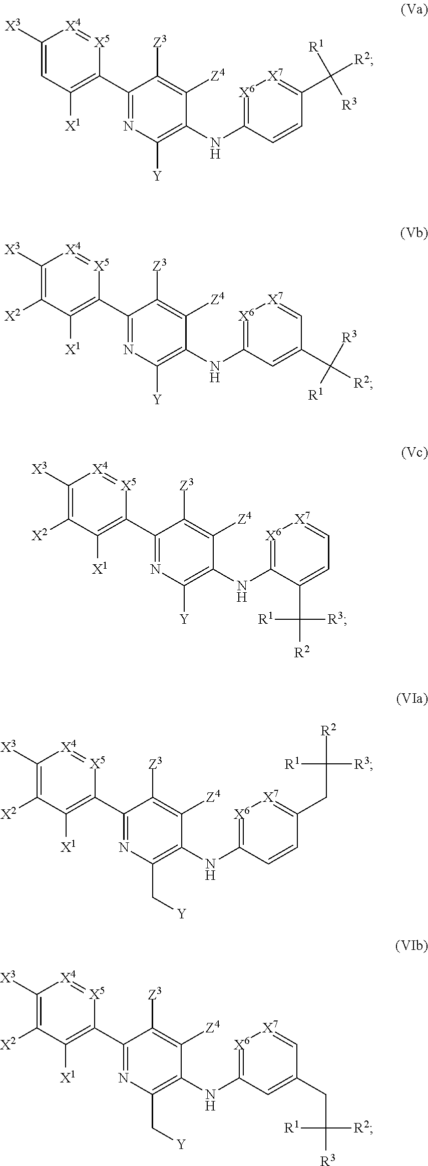

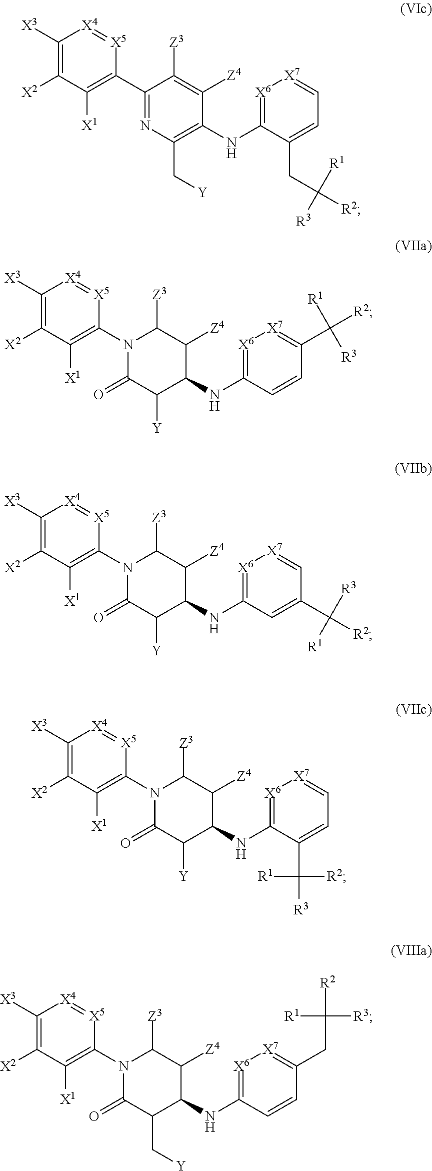

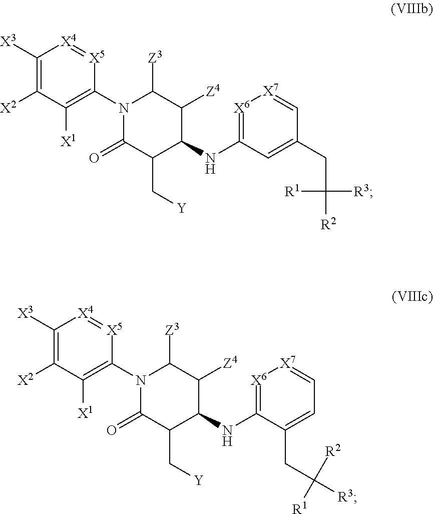

a resonance structure thereof, or
a pharmaceutically acceptable salt or aqueous solution or dispersion thereof,
wherein each of R¹, R², and R³ individually are a halogen, hydrogen, hydroxyl, amine (NH₂),

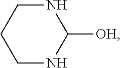

or absent;
wherein X¹, X², and X³ individually are a hydrogen, halogen, O, or C═O;
wherein X⁴, X³, X⁶, and X⁷ individually are C or N, preferably wherein one of X⁴ or X⁵ is N
   when Z¹ or Z³ is S or O, and/or preferably wherein one of X⁶ or X⁷ is N when Z² or Z⁴ is S
   or O,
wherein at least two of X¹, X², and X³ are hydrogen;
wherein Y is HC≡N, CH₃, CH₃—CH₄, C═O, hydrogen, absent; and
wherein Z¹, Z², Z³, and Z⁴ individually are S, O, amine (NH₂), ═S, ═O, =amine (═NH₂), —SH, —OH, —H, or a protonated S, O, amine (NH₂).

The compounds can also include resonance structures, such as shown in the following formulae Va:

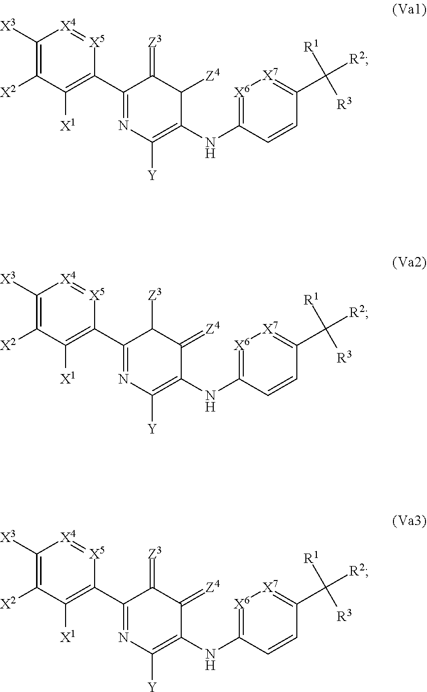

or a pharmaceutically acceptable salt or aqueous solution or dispersion thereof,
wherein each of R¹, R², and R³ individually are a halogen, hydrogen, hydroxyl, amine (NH₂),

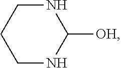

or absent;
wherein X¹, X², and X³ individually are a hydrogen, halogen, O, or C═O;
wherein X⁴, X⁵, X⁶, and X⁷ individually are C or N, and preferably wherein one of X⁴ or X³ is N when Z³ is S or O, and/or preferably wherein one of X⁶ or X⁷ is N when Z⁴ is S or O;
wherein at least two of X¹, X², and X³ are hydrogen;
wherein Y is HC≡N, CH₃, CH₃—CH₄, C═O, hydrogen, absent; and
wherein Z³, and Z⁴ individually are S, O, amine (NH₂), ═S, ═O, =amine (═NH₂), —SH, —OH, —H, or a protonated S, O, amine (NH₂).

Additional examples of resonance structures are shown with exemplary formulae VIIa, VIIb, VIIIa, and VIIIb:

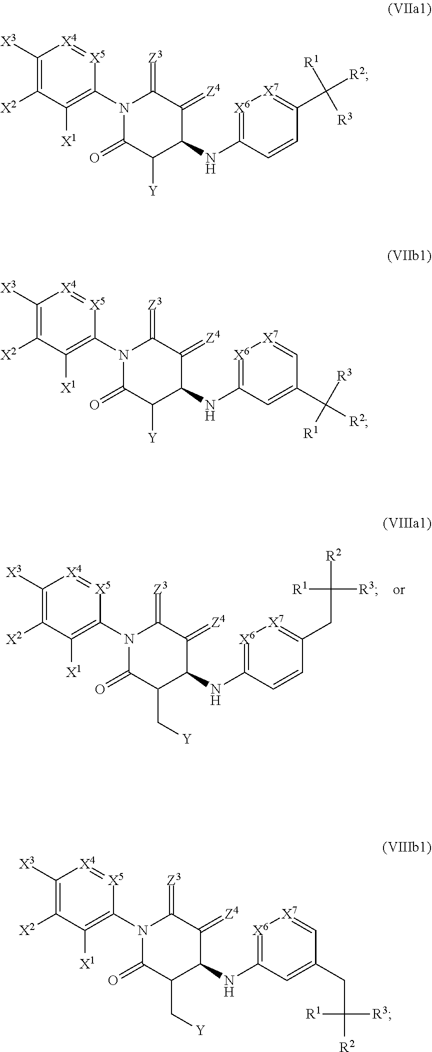

other resonance structures, or
a pharmaceutically acceptable salt or aqueous solution or dispersion thereof,
wherein each of R¹, R², and R³ individually are a halogen, hydrogen, hydroxyl, amine (NH₂),

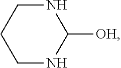

or absent;
wherein X¹, X², and X³ individually are a hydrogen, halogen, O, or C═O;
wherein X⁴, X⁵, X⁶, and X⁷ individually are C or N, and preferably wherein one of X⁴ or X⁵ is N when Z³ is S or O, and/or preferably wherein one of X⁶ or X⁷ is N when Z⁴ is S or O;
wherein at least two of X¹, X², and X³ are hydrogen;
wherein Y is HC≡N, CH₃, CH₃—CH₄, C═O, hydrogen, absent; and
wherein Z³, and Z⁴ individually are S, O, amine (NH₂), —SH, —OH, or —H.

In additional embodiments the compound of Formula Ia, Ib, or Ic is one of the following formulae:

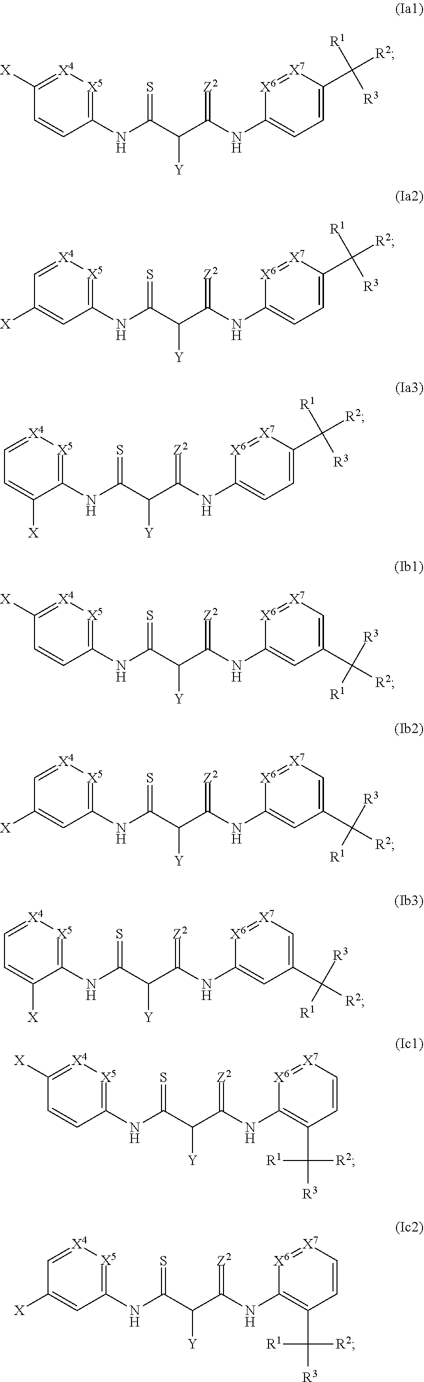

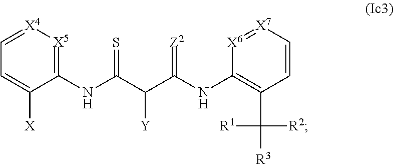

a resonance structure thereof, or
a pharmaceutically acceptable salt or aqueous solution or dispersion thereof,
wherein each of R¹, R², and R³ individually are a halogen, hydrogen, hydroxyl, amine (NH₂),

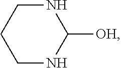

or absent;
wherein X is a hydrogen, halogen, O, or C═O;
wherein X⁴, X³, X⁶, and X⁷ individually are C or N, and preferably wherein one of X⁴ or X⁵ is N, and/or preferably wherein one of X⁶ or X⁷ is N when Z² is S or O;
wherein Y is HC≡N, CH₃, CH₃—CH₄, C═O, hydrogen, absent; and
wherein Z² is S, O, amine (NH₂), —SH, —OH, or —H.

In additional embodiments the compound of Formula IIa, IIb, or IIc is one of the following formulae:

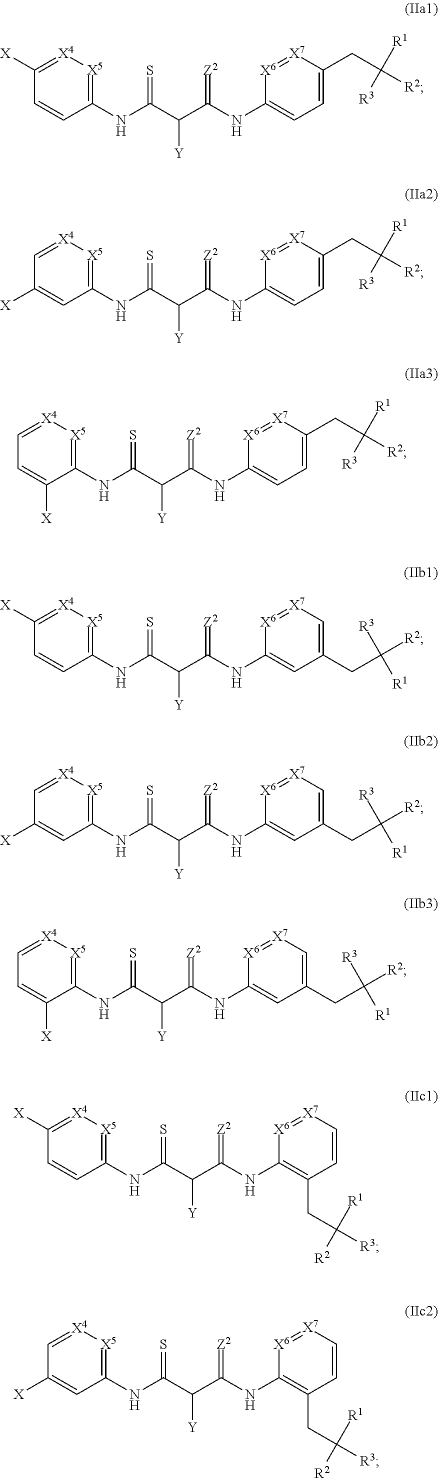

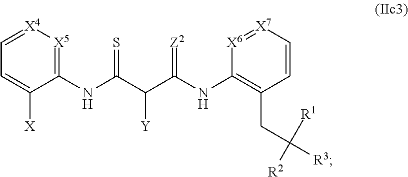

a resonance structure thereof, or
a pharmaceutically acceptable salt or aqueous solution or dispersion thereof,
wherein each of R¹, R², and R³ individually are a halogen, hydrogen, hydroxyl, amine (NH₂),

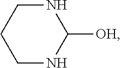

or absent;
wherein X is a hydrogen, halogen, O, or C═O;
wherein X⁴, X³, X⁶, and X⁷ individually are C or N, and preferably wherein one of X⁶ or X⁷ is N when Z² is S or O,
wherein Y is HC≡N, CH₃, CH₃—CH₄, C═O, hydrogen, absent; and
wherein Z² is S, O, amine (NH₂), —SH, —OH, or —H.

In additional embodiments the compound of Formula IIIa, IIIb, IIIc, IVa, IVb, or IVc is one of the following formulae:

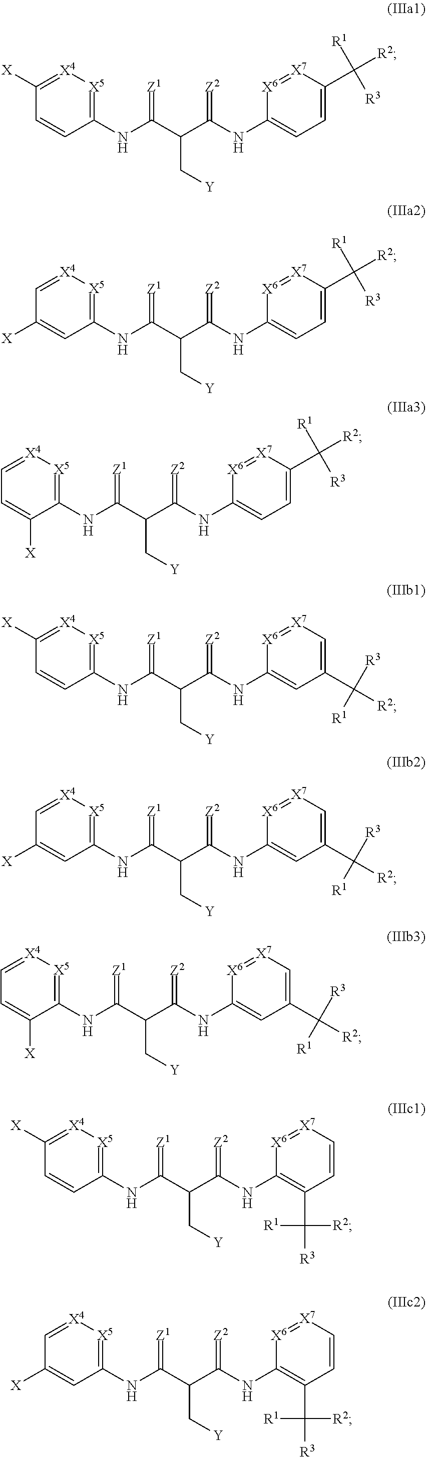

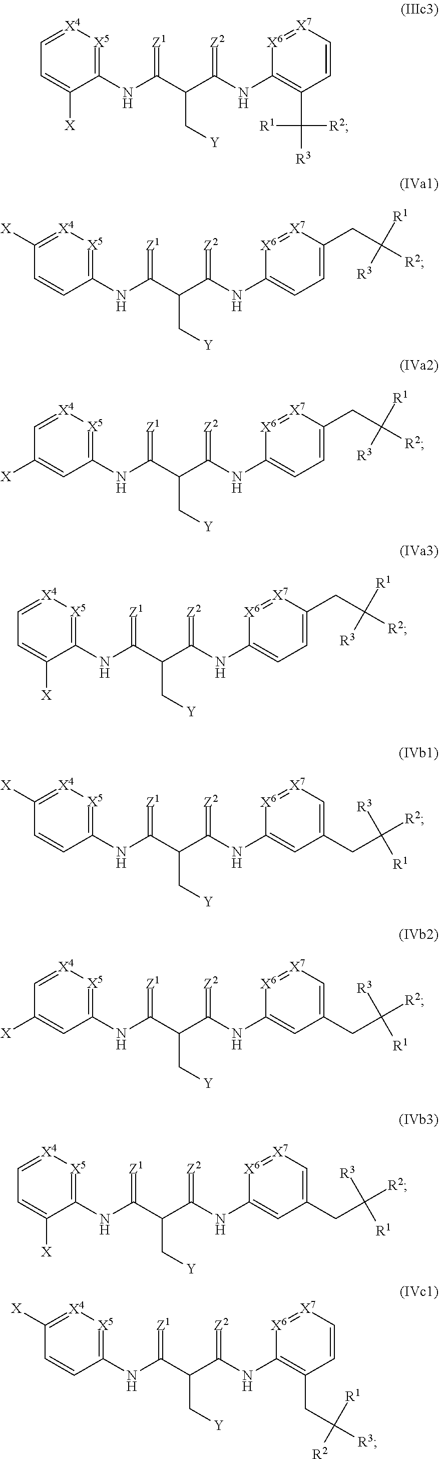

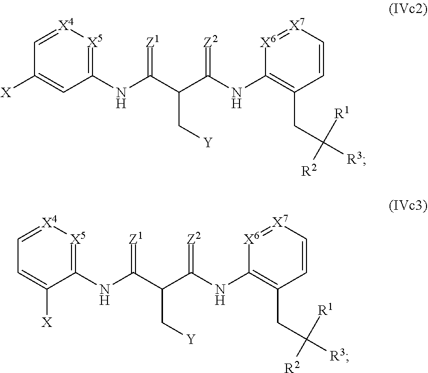

a resonance structure thereof, or
a pharmaceutically acceptable salt or aqueous solution or dispersion thereof,
wherein each of R¹, R², and R³ individually are a halogen, hydrogen, hydroxyl, amine (NH₂),

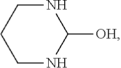

or absent;
wherein X is a halogen, O, or C═O;
wherein X⁴, X³, X⁶, and X⁷ individually are C or N, and wherein preferably one of X⁴ or X³ is N when Z¹ is S or O, and/or wherein preferably one of X⁶ or X⁷ is N when Z² is S or O,
wherein Y is HC≡N, CH₃, CH₃—CH₄, C═O, hydrogen, or absent; and
wherein Z¹ and Z² individually are S, O, amine (NH₂), —SH, —OH, or —H.

In additional embodiments the compound of Formula Va, Vb, Vc, VIa, VIb, VIc, VIIa, VIIb, VIIc, Villa, VIIIb, or VIIIc is one of the following formulae

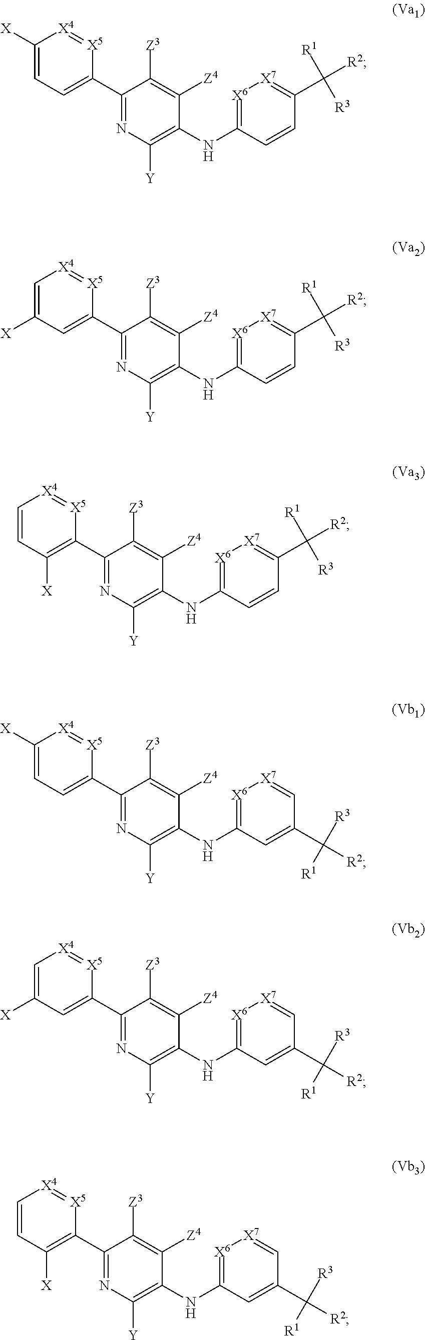

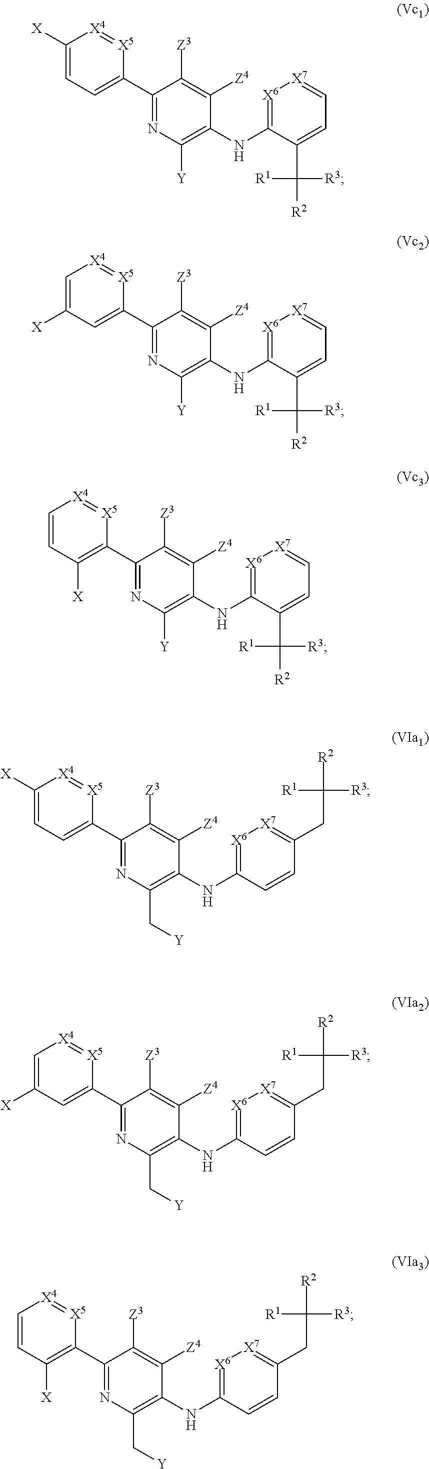

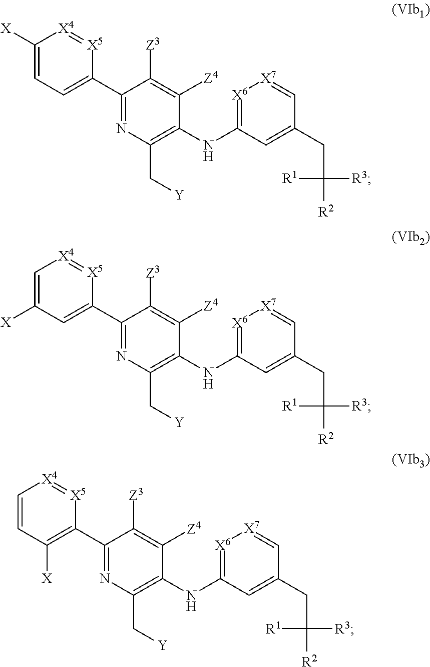

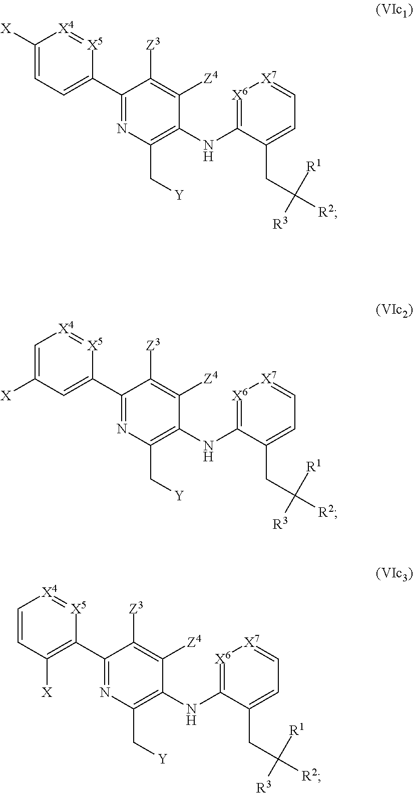

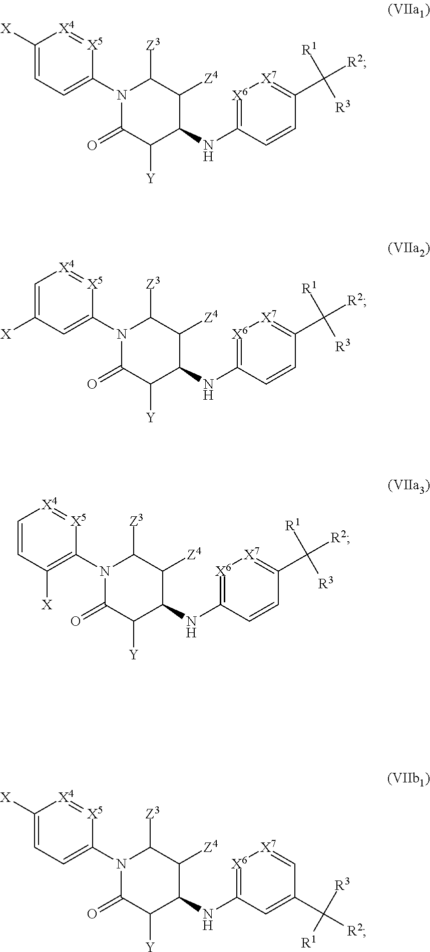

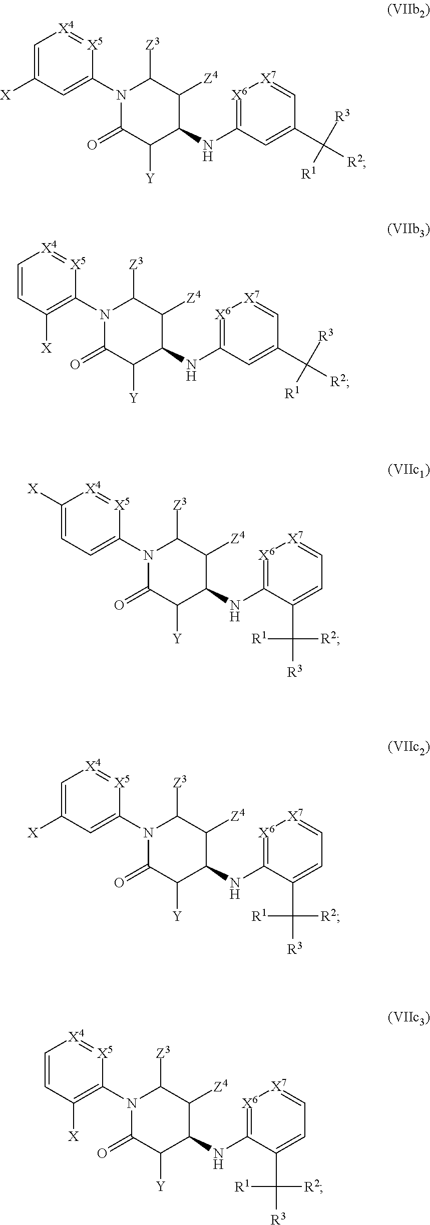

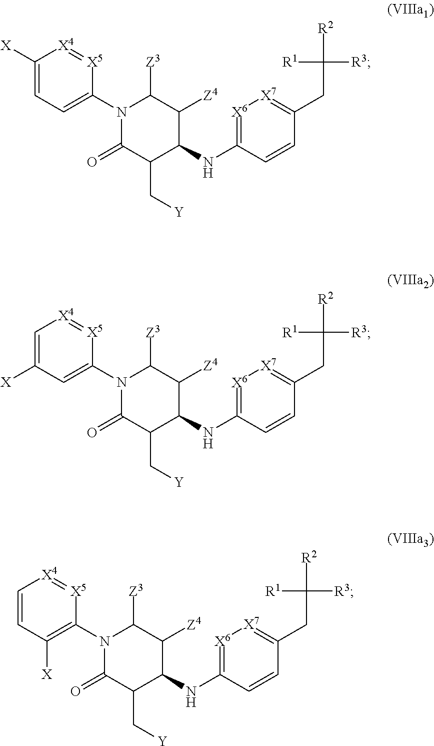

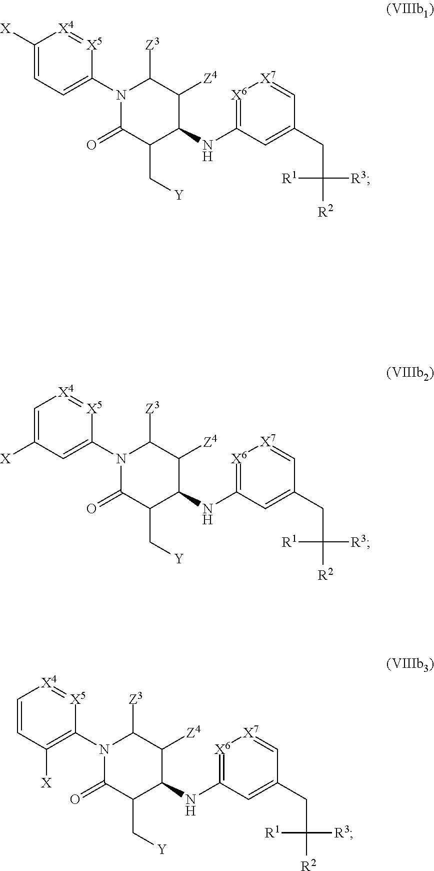

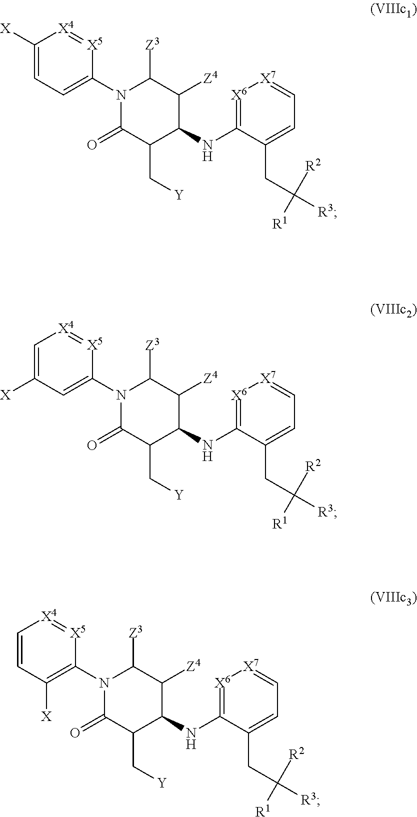

a resonance structure thereof, or
a pharmaceutically acceptable salt or aqueous solution or dispersion thereof,
wherein each of R¹, R², and R³ individually are a halogen, hydrogen, hydroxyl, amine (NH₂),

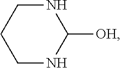

or absent;
wherein X is a halogen, O, or C═O;
wherein X⁴, X⁵, X⁶, and X⁷ individually are C or N, and preferably wherein one of X⁴ or X⁵ is N when Z³ is S or O, and/or preferably wherein one of X⁶ or X⁷ is N when Z⁴ is S or O;
wherein Y is HC≡N, CH₃, CH₃—CH₄, C═O, hydrogen, or absent; and
wherein Z³ and Z⁴ individually are S, O, amine (NH₂), ═S, ═O, =amine (═NH₂), —SH, —OH, —H, or
   a protonated S, O, amine (NH₂).

In additional embodiments the compound of any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, or a pharmaceutically acceptable salt or aqueous solution or dispersion thereof, has the formula wherein R¹ is a hydroxyl and R² and R³ are each F, Br, Cl, or I, wherein X¹, X², and X³ is hydrogen, Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, or a pharmaceutically acceptable salt or aqueous solution or dispersion thereof, has the formula wherein R³ is a hydroxyl and R¹ and R² are each F, Br, Cl, or I, wherein X¹, X², and X³ is hydrogen, Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, or a pharmaceutically acceptable salt or aqueous solution or dispersion thereof, has the formula wherein each of R¹, R², and R³ is independently F, Br, Cl, I, hydrogen, or absent, wherein X¹, X², and X³ is independently hydrogen, Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, or a pharmaceutically acceptable salt or aqueous solution or dispersion thereof, has the formula wherein R² is

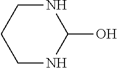

and R¹ and R³ are each F, Br, Cl, or I, wherein X¹, X², X³ is hydrogen, Cl, Br, F, I, O, or C═O, wherein X⁴, X⁵, X⁶, and X⁷ individually are C or N, wherein Y is HC≡N, CH₃, CH₃—CH₄, C═O, hydrogen or absent.

In additional embodiments the compound of any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, a resonance structure thereof, or a pharmaceutically acceptable salt or aqueous solution or dispersion thereof, has the formula wherein R¹ or R³ is-NH₂, wherein X¹, X², and X³ is hydrogen, Cl, Br, F, I, O, or C—O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae Ia₍₁₋₃₎, Ib₍₁₋₃₎, or Ic₍₁₋₃₎ has the formula wherein: (i) R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (ii) R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iii) each of R¹, R², and R³ is F, Br, Cl, I, hydrogen, or absent, wherein X is Cl, Br, F, I, O, or C—O, wherein X⁴, X⁵, X⁶, and X⁷ individually are C or N, and wherein Y is HC≡N, CH₃, CH₃—CH₄, C═O, hydrogen or absent; (iv) R² is

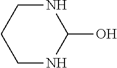

and R¹ and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; or (v) R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae IIa₍₁₋₃₎, IIb₍₁₋₃₎ or IIc₍₁₋₃₎ has the formula wherein: (i) R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (ii) R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iii) each of R¹, R², and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iv) R² is

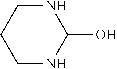

and R¹ and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; or (v) R¹ or R³ is-NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae IIIa₍₁₋₃₎, IIIb₍₁₋₃₎, IIIc₍₁₋₃₎, IVa₍₁₋₃₎, or IVb₍₁₋₃₎ has the formula wherein: (i) R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (ii) R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iii) each of R¹, R², and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iv) R² is

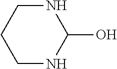

and R¹ and R³ are each F, Br, Cl, or I, wherein X is Cl, Br, F, I, O, or C—O, wherein X⁴, X⁵, X⁶, and X⁷ individually are C or N, and wherein Y is HC≡N, CH₃, CH₃—CH₄, C═O, hydrogen or absent; or (v) R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

In additional embodiments the compound of any one of Formulae Va₍₁₋₃₎, Vb₍₁₋₃₎, Vc₍₁₋₃₎, VIa₍₁₋₃₎, VIb₍₁₋₃₎, VIC₍₁₋₃₎, VIIa₍₁₋₃₎, VIIb₍₁₋₃₎, VIIc₍₁₋₃₎, VIIIa₍₁₋₃₎, VIIIb₍₁₋₃₎, VIIIc₍₁₋₃₎, has the formula wherein: (i) R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (ii) R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; (iii) each of R¹, R², and R³ is F, Br, Cl, I, hydrogen, or absent, wherein X is Cl, Br, F, I, O, or C—O, wherein X⁴, X⁵, X⁶, and X⁷ individually are C or N, and wherein Y is HC≡N, CH₃, CH₃—CH₄, C═O, hydrogen or absent; (iv) R² is

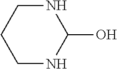

and R¹ and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; or (v) R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

### Pharmaceutical Compositions

According to an embodiment, a pharmaceutical composition is provided. A pharmaceutical composition according to the present disclosure includes a compound that competitively inhibit a P1B-type heavy metal ATPase as described herein and a pharmaceutically acceptable carrier.

By “pharmaceutical composition” the inhibitor(s) of P1B-type heavy metal ATPase of the present disclosure provide the therapeutically or biologically active agent for formulation into a suitable delivery means for administration to a subject. For the purposes of this invention, pharmaceutical compositions suitable for delivering the inhibitor(s) of P1B-type heavy metal ATPase can include, e.g., tablets, gel caps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of the aforementioned formulations can be prepared by well-known and accepted methods of art.

In an aspect, the pharmaceutical compositions comprise at least one of the inhibitors of P1B-type heavy metal ATPase and a pharmaceutically acceptable carrier or excipient. Examples of suitable pharmaceutically acceptable carriers or excipients that can be used in said pharmaceutical compositions include, but are not limited to, sugars (e.g., lactose, glucose or sucrose), starches (e.g., corn starch or potato starch), cellulose or its derivatives (e.g., sodium carboxymethyl cellulose, ethyl cellulose or cellulose acetate), oils (e.g., peanut oil, cottonseed oil, safflower oil, sesame oil, olive oil, corn oil or soybean oil), glycols (e.g., propylene glycol), buffering agents (e.g., magnesium hydroxide or aluminum hydroxide), agar, alginic acid, powdered tragacanth, malt, gelatin, talc, cocoa butter, pyrogen-free water, isotonic saline, Ringer's solution, ethanol, phosphate buffer solutions, lubricants, coloring agents, releasing agents, coating agents, sweetening, flavoring or perfuming agents, preservatives, or antioxidants.

The term “excipient” refers to additives and stabilizers typically employed in the art (all of which are termed “excipients”), including for example, buffering agents, stabilizing agents, preservatives, isotonifiers, non-ionic detergents, antioxidants and/or other miscellaneous additives. Stabilizers refer to a broad category of excipients which can range in function from a bulking agent to an additive which solubilizes the inhibitor of P1B-type heavy metal ATPase or helps to prevent denaturation of the same. Additional conventional excipients include, for example, fillers (e.g., starch), chelating agents (e.g., EDTA), antioxidants (e.g., ascorbic acid, methionine, vitamin E) and cosolvents.

The term “carrier” refers to a diluent, adjuvant, excipient, or vehicle with which the pharmaceutical composition is administered. Such pharmaceutical carriers are illustratively sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions are optionally employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, also contains wetting or emulsifying agents, or pH buffering agents. These compositions optionally take the form of solutions, suspensions, emulsion, tablets, pills, capsules, powders, sustained release formulations and the like. The composition is optionally formulated as a suppository, with traditional binders and carriers such as triglycerides. Oral formulation illustratively includes standard carriers such as pharmaceutical grades of mannitol, lactose, starch, magnesium stearate, sodium saccharine, cellulose, magnesium carbonate, etc.

Pharmaceutical compositions according to the disclosure may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED50)); (ii) a narrow absorption window in the gastro-intestinal tract; or (iii) a short biological half-life, so that frequent dosing during a day is required in order to sustain a therapeutic level. One skilled in the art will ascertain compositions for controlled or extended release of the pharmaceutical composition. In an aspect, controlled release can be obtained by controlled release compositions and coatings which are known to those of skill in the art.

Methods for Preventing and/or Treating a Health Condition and/or Disease in a Subject

In an aspect, methods for preventing and/or treating a health condition and/or disease in a subject in need thereof are provided. In an embodiment, the method comprises administering a therapeutically effective amount of an inhibitor of a heavy metal transporter to the subject. In some embodiments, the inhibitor of the heavy metal transporter comprises an inhibitor of ATP7A and/or ATP7B.

In an embodiment, the method comprises administering to the subject a therapy comprising a therapeutically effective amount of the compounds described herein. In an embodiment, the method comprises administering to the subject a pharmaceutical composition comprising the compound and a pharmaceutically acceptable carrier.

In some embodiments, the compound administered to the subject competitively inhibits a P1B-type heavy metal ATPase. The P1B-type heavy metal ATPase may comprise a P1B-type copper ATPase. In an embodiment, the compound specifically binds to an intramembraneous pocket of P1B-type copper ATPase. In a further embodiment, the P1B-type copper ATPase is ATP7A and/or ATP7B, and the administered compound competitively inhibits ATP7A and/or ATP7B by blocking entry of copper into ATP7A and/or ATP7B. In some embodiments, the compound disrupts transmembrane copper transport.

In some embodiments, competitive inhibition of ATP7A and/or ATP7B disrupts delivery of copper to at least one lysyl oxidase (LOX), thereby inhibiting activity of the at least one LOX. In an embodiment, competitive inhibition of ATP7A comprises inhibition of LOX or LOXL1-4 enzyme activity in cancer cells, thereby suppressing cancer tumorigenesis, cancer cell migration, and cancer cell metastasis in the subject.

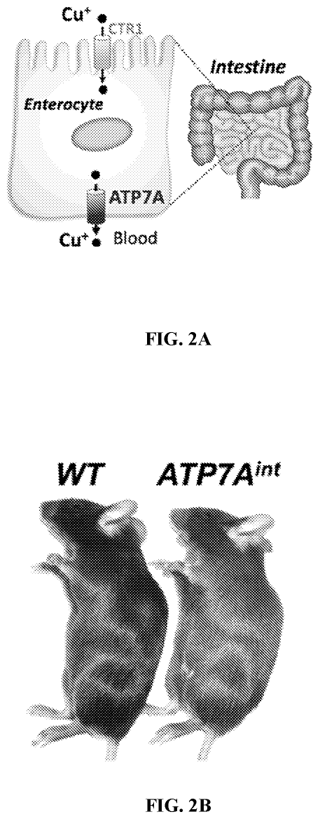

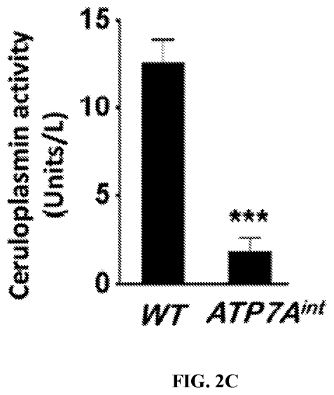

In some embodiments, competitive inhibition of ATP7A and/or ATP7B inhibits the copper-dependent activity of the tyrosinase enzymes, thereby inhibiting melanogenesis (See FIG. 2B).

In some embodiments, the therapeutically effective amount of the compound or the pharmaceutical composition is from about 10 nM to about 1 mM. In some embodiments, the therapeutically effective amount of the compound or the pharmaceutical composition is from about 10 nM to about 500 μM, from about 10 nM to about 400 μM, from about 10 nM to about 300 μM, from about 10 nM to about 200 μM, from about 10 nM to about 150 μM, from about 10 nM to about 100 μM, from about 10 nM to about 500 nM, or any range therein.

Dose ranges can be adjusted as necessary for the treatment of individual patients and according to the specific condition treated and the type of delivery for the administration of the compositions. Any of a number of suitable pharmaceutical formulations may be utilized as a vehicle for the administration of the compositions of the present disclosure and maybe a variety of administration routes are available. The particular mode selected will depend of course, upon the particular formulation selected, the severity of the disease, disorder, or condition being treated, and the dosage required for therapeutic efficacy. The methods of this disclosure, generally speaking, may be practiced using any mode of administration that is medically acceptable, meaning any mode that produces effective levels of the active compounds without causing clinically unacceptable adverse effects. Such modes of administration include oral, rectal, topical, nasal, transdermal or parenteral routes and the like. Accordingly, the formulations of the invention include those suitable for oral, rectal, topical, buccal, parenteral (e.g., subcutaneous, intramuscular, intradermal, inhalational or intravenous) and transdermal administration, although the most suitable route in any given case will depend on the nature and severity of the condition being treated and on the nature of the particular active product used.

In an embodiment, the therapy is administered by a variety of administration methods, including, but not limited to, oral administration, transdermal administration, topical administration, ocular administration, sublingual administration, parenteral administration, aerosol administration, administration via inhalation, intravenous or intra-arterial administration, local administration via injection or cannula, vaginal administration and/or rectal administration.

In general, the formulations of the disclosure are prepared by uniformly and intimately mixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for transdermal administration may also be presented as medicated bandages or discrete patches adapted to remain in intimate contact with the epidermis of the recipient for a prolonged period of time. Formulations suitable for transdermal administration may also be delivered by iontophoresis (passage of a small electric current to “inject” electrically charged ions into the skin) through the skin. For this, the dosage form typically takes the form of an optionally buffered aqueous solution of the active compound.

Formulations of the present disclosure suitable for parenteral administration may conveniently comprise sterile aqueous preparations of the active compound, which preparations are preferably isotonic with the blood of the intended recipient. These preparations may be administered by means of subcutaneous, intravenous, intramuscular, inhalational or intradermal injection. Such preparations may conveniently be prepared by mixing the compound with water or a glycerin buffer and rendering the resulting solution sterile and isotonic with the blood. Alternately, the extracts, fractions thereof or compounds thereof can be added to a parenteral lipid solution.

Formulations suitable for rectal administration are preferably presented as unit dose suppositories. These may be prepared by mixing the active compound with one or more conventional solid carriers, for example, cocoa butter, and then shaping the resulting mixture.

The methods disclosed herein may be used in the treatment of any health condition, disease, or pathology in which copper and/or copper metabolism plays a role in disease pathology or progression. In an embodiment, the disease treated and/or prevented includes any disease in which copper and/or P1B-type heavy-metal ATPases contribute to the disease pathology.

In an embodiment, the method is used in the prevention and/or treatment of cancer and/or fibrotic diseases. In some embodiments, the cancer comprises blood cancer, carcinoma, sarcoma, mesothelioma, colorectal cancer, pancreatic cancer, head and neck cancer, skin cancer, gastric cancer, breast cancer, prostate cancer, thyroid cancer, endometrial cancer, ovarian cancer, lung cancer, hepatocellular carcinoma, and/or kidney renal papillary cell carcinoma. In a further embodiment, the cancer is head and neck cancer comprising esophageal cancer. In a further embodiment, the cancer is pancreatic cancer comprising pancreatic ductal adenocarcinoma.

In an embodiment, the compound or pharmaceutical composition is administered as an antibiotic. The compound may be administered with a therapeutically effective amount of silver and/or copper, or in conjunction with a therapy that alters cellular concentrations of copper (eg., a copper chelator or ionophore). In some embodiments, a therapeutically effective amount of silver and/or copper is from about 1 nM to about 1 mM, from about 1 nM to about 750 μM, from about 1 nM to about 500 μM, from about 1 nM to about 400 μM, from about 1 nM to about 300 μM, from about 1 nM to about 200 μM, from about 1 nM to about 100 μM, or any range therein. A person having skill in the art will recognize the dosing will vary based on the desired use, the pathogen being treated, and the form of administration. For example, higher doses may be desirable and tolerated if the administration is topical. In some embodiments, the compound augments bactericidal or fungicidal properties of the silver and/or copper.

In some embodiments, the disease or condition treated and/or prevented comprises tissue scarring and/or tissue fibrosis; diseases of copper disturbance including Menkes disease, Wilson disease, Alzheimer's disease, Amyotrophic lateral sclerosis, Parkinson's disease, Huntington's disease, Creutzfeldt Jakob disease, MEDNIK syndrome; infection and/or microbial resistance to silver or copper; hyperpigmentation or pigmentation disorders such as postinflammatory hyperpigmentation, melasma, solar lentigines (sun spots), ephelides (freckles), café au lait macules, vitiligo, pityriasis alba, tinea versicolor, and postinflammatory hypopigmentation.

Methods for Augmenting Chemotherapy Drug Efficacy and/or Treating Chemotherapy Drug Resistance in a Subject

In an aspect, methods for augmenting chemotherapy drug efficacy or preventing and/or treating chemotherapy drug resistance in a subject in need thereof are provided. In an embodiment, the method comprises administering to the subject a compound or pharmaceutical composition described herein.

In some embodiments, the chemotherapy drug treatments and/or chemotherapy drug resistance comprises cisplatin resistance, vincristine resistance, paclitaxel resistance, SN-38 resistance, etoposide resistance, doxorubicin resistance, mitoxantrone resistance, and/or 7-ethyl-10-[4-(1-piperidino)-1-piperidino] carbonyloxycamptothecin (CPT-11) resistance. In an embodiment, the chemotherapy resistance is cisplatin resistance or doxorubicin resistance.

### Methods for Modulating Copper Transport

Copper transporting P1B-type ATPases are found in every type of living organism including archaebacteria, bacteria, protists, fungi, plants and animals. Notably, the MKV3-binding pocket is highly conserved among all P1B-type ATPases and thus the compounds disclosed herein may be applied to a wide array of organisms to modulate any biological process that is dependent on copper transport via a P1B-type ATPase. For example, fruit ripening in plants is controlled by the ethylene receptor which receives copper from a P1B-type ATPase in a manner akin to tyrosinase metallation in animal cells. Thus, the compounds and methods disclosed herein can be used to modulate non-pathological biological processes as well as disease-related or pathological processes.

In some embodiments, a method for modulating copper transport in a subject comprises administering to the subject a compound described herein. In some embodiments, the compound competitively inhibits a P1B-type heavy metal ATPase, including P1B-type copper ATPase. In some embodiments, the compound specifically binds to an intramembraneous pocket of the P1B-type copper ATPase.

In some embodiments, the P1B-type copper ATPase is ATP7A and/or ATP7B. In an embodiment, the compound competitively inhibits the P1B-type copper ATPase by blocking entry of copper into the P1B-type copper ATPase.

Embodiments of the present disclosure are further defined in the following non-limiting Examples. It should be understood that these Examples, while indicating certain embodiments of the disclosure, are given by way of illustration only. From the above discussion and these Examples, one of ordinary skill in the art can ascertain the essential characteristics of this disclosure and, without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those of ordinary skill in the art from the foregoing description. Such modifications are also intended to fall within the scope of the appended claims.

### EXAMPLES

### Example 1

### Identification of ATP7A as a Potential Therapeutic Target

Copper (Cu) is required for growth and development in all multicellular organisms. In the absence of sufficient levels of copper, pathology can result. In humans, this is illustrated by Menkes disease, a pediatric disorder of copper deficiency caused by mutations in the copper transporter, ATP7A, which is responsible for transporting copper across the basolateral membrane of intestinal enterocytes into the blood (FIG. 1, FIG. 2A).

Previous murine models have shown that intestine-specific knockout of the ATP7A gene (ATP7A^{int} mice) results in lethality shortly after birth. However, ATP7A^{int} mice can be rescued by a single injection of CuCl₂ if given within a week of birth. The rescued ATP7A^{int} mice at maturity exhibit normal size, behavior and life-expectancy compared to wild type mice. However, their whole-body copper status remains low throughout their life as evidenced by a lighter coat color (FIG. 2B), due to reduced activity of tyrosinase, a Cu-dependent enzyme required for melanin synthesis. Additionally, the activity of serum ceruloplasmin, a biomarker of copper status, was reduced in mature ATP7A^{int} mice to approximately 15% of wild type levels (FIG. 2C). This result is similar to target levels of ceruloplasmin in cancer patients undergoing copper depletion therapy and thus provides proof of concept that a small molecule that blocks ATP7A function in the intestine could be used to deplete systemic copper levels in patients with cancer or other condition such as Wilson disease in which copper depletion is desired.

In addition to controlling copper export from intestinal epithelial cells into the circulation, ATP7A also regulates the export of copper in most cells throughout the body. If copper concentrations become elevated, ATP7A traffics from the trans-Golgi network to the plasma membrane to facilitate copper export and restore copper homeostasis.

Previous studies have demonstrated the importance of ATP7A in primary tumor growth in mice. In syngeneic mouse models, ATP7A deletion reduces primary tumor growth of 4T1 mammary carcinoma cells and LLC lung carcinoma cells. Other studies have shown that ATP7A is a major contributor to the growth and survival of cancer cells carrying mutations in the KRAS gene, a major oncogenic driver in about 32% of lung cancers, about 40% of colorectal cancers, and about 85% of pancreatic cancers. Other studies have shown that ATP7A or ATP7B function in resistance to frontline cancer chemotherapy agents including cisplatin, vincristine, paclitaxel, SN-38, etoposide, doxorubicin, mitoxantrone, and 7-ethyl-10-[4-(1-piperidino)-1-piperidino] carbonyloxycamptothecin (CPT-11). ATP7A is also important in promoting the growth of blood vessels (angiogenesis) by limiting the degradation of the VEGFR2 receptor. An inhibitor of ATP7A/B would be expected to prevent or reverse the development of chemotherapy drug resistance in cancer cells and may provide therapeutic benefit in cancer patients.

Based on these results, ATP7A was selected for further study as a therapeutic target for cancer models.

### Example 2

### ATP7A is Required to Metalate the LOX Family of Oncogenic Enzymes

The family of Cu-dependent lysyl oxidases (LOX) plays a significant role in cancer metastasis. Several oncogenic mechanisms have been attributed to LOX proteins including the activation of tumor cell migration via focal adhesion kinase and the creation of the pre-metastatic niche in distant organs that promotes seeding of tumor cells. Although there have been ongoing efforts to develop inhibitors of LOX family members, a major challenge has been finding an inhibitor of all LOX proteins.

It was hypothesized that inhibiting copper incorporation into all LOX enzymes may be an effective strategy to attenuate LOX-dependent metastasis. Since each LOX family member shares a functional requirement for copper and is secreted from cells, it was believed that ATP7A activity may be required to metalate these enzymes within the secretory pathway.

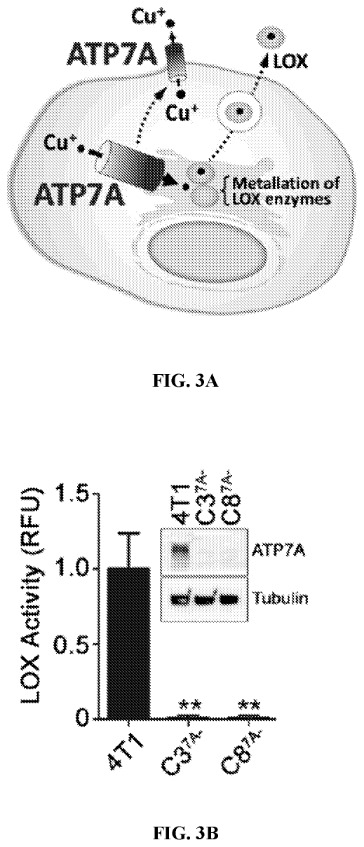

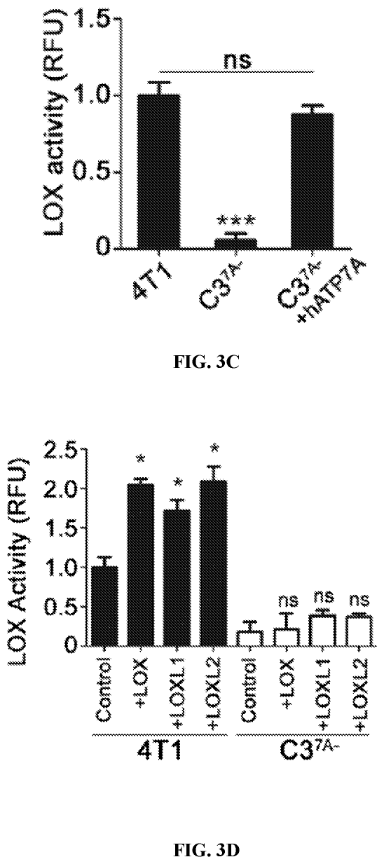

To test this hypothesis, CRISPR-Cas9 was used to generate an out-of-frame deletion in the ATP7A gene in 4T1 cells, a metastatic cell model of breast carcinoma. The 4T1 model is a highly metastatic triple-negative breast cancer cell line (i.e., ER-, PR-, HER2-) lacking the estrogen receptor, prolactin receptor and the epidermal growth factor receptor HER2. The 4T1 cells readily form primary tumors when injected orthotopically into the mammary glands of Balb/C mice, and closely model aggressive forms of human breast cancer by metastasizing to lymph nodes, lung, bone and liver. In two independent clones lacking ATP7A (C3^{7A} and C8^{7A-}), it was observed that a significant reduction of LOX activity was secreted into the medium compared to wild type 4T1 cells (FIG. 3B). This was not attributable to off-target effects of CRISPR-Cas9 because LOX activity was fully restored by transfection of a human ATP7A expression construct (FIG. 3C). Moreover, the addition of supraphysiological copper to the media was found to restore LOX activity in C37A-cells. Together, these observations indicate that the inhibitory effects of ATP7A deletion are due to a failure of LOX metalation.

To investigate the effects of ATP7A deletion on specific LOX proteins, wild type 4T1 and C3^{7A-} cells were transfected with plasmids encoding one of three different LOX family members (LOX, LOXL1 and LOXL2) together with a GFP plasmid to control for transfection efficiency. After 24 hours the media were collected, and LOX activity was measured as a function of cellular GFP expression. For wild type cells, each LOX plasmid produced elevated LOX activity in the medium relative to cells transfected with the GFP vector alone (FIG. 3D). In contrast, none of the LOX plasmids produced a significant increase in LOX activity in C37A-cells (FIG. 3D).

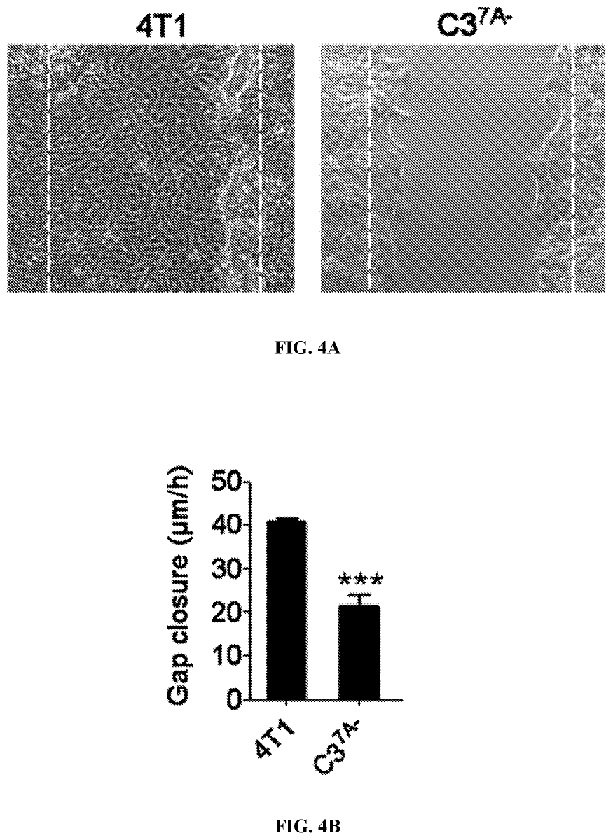

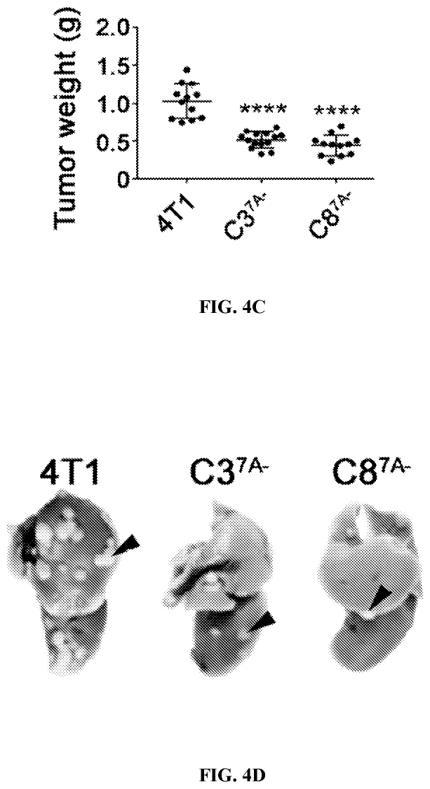

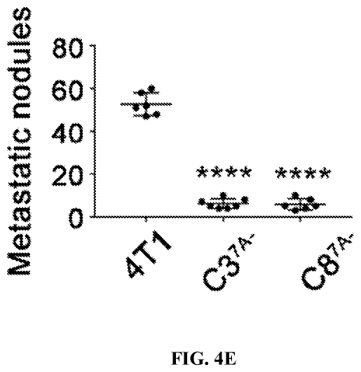

Furthermore, it was demonstrated that ATP7A deletion inhibits tumor cell migration in vitro. An important mechanism of LOX-mediated metastasis is the activation of focal adhesion kinase, FAK1, a key regulator of tumor cell migration. FAK1 phosphorylation is known to regulate the assembly of proteins, including vinculin, at focal adhesion sites. The loss of ATP7A significantly reduces phosphorylation of FAK1 compared to wild type 4T1 cells, which causes an increase in vinculin-positive focal adhesions. Consistent with these changes, the loss of ATP7A resulted in a significant reduction in cell motility as demonstrated by reduced gap closure in an in vitro scratch assay (FIGS. 4A and 4B). These studies demonstrate that ATP7A is necessary for LOX-dependent pathways of cell migration in vitro.

### Example 3

### Validation of ATP7A as an Oncogenic Target In Vivo

4T1 cells undergo LOX-dependent metastasis from the primary tumor to the lungs. To test the effect of ATP7A deletion on primary tumor growth and metastasis, 4T1 cells were injected into the 4th inguinal mammary fat pads of female BALB/c mice. After 4 weeks, primary tumor growth and metastatic lung nodules were quantified. Both the C37A− and C87A− tumors lacking ATP7A were significantly smaller than wild type tumors (FIG. 4C). Importantly, there were markedly fewer metastatic lung nodules in mice bearing C3^{7A-} and C8^{7A-} tumors compared to mice bearing wild type tumors (FIGS. 4D and 4E). This result validates ATP7A as a novel target for generating therapeutic levels of systemic copper deficiency, and for attenuating the growth and metastasis of cancer cells.

### Example 4

### In Silico Design of ATP7A Inhibitors

P-type ATPases are a large superfamily of transporters found in all kingdoms of life that pump cations or phospholipids across membranes using the energy derived from ATP hydrolysis. ATP7A and ATP7B are the only mammalian examples of P1B-type ATPases, an evolutionarily distinct subgroup of P-type ATPases that transport Cu¹⁺ ions (or other heavy metals in certain bacteria). Inhibitors of non-P1B ATPases have been described. For example, omeprazole is an inhibitor of the gastric H⁺/K⁺ ATPase, ouabain is an inhibitor of Na²⁺/K⁺ ATPase, and thapsigargin is an inhibitor of the SERCA Ca²⁺-ATPase. However, previously there were no known inhibitors of any P1B-type ATPase or any other type of heavy metal transporter.

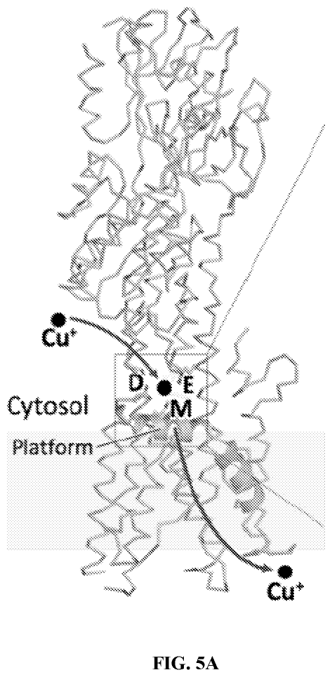

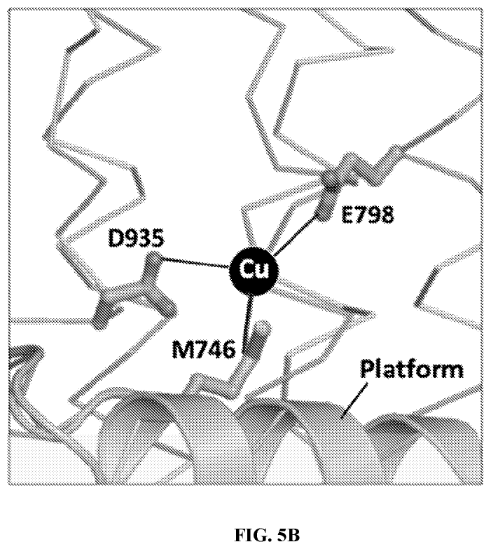

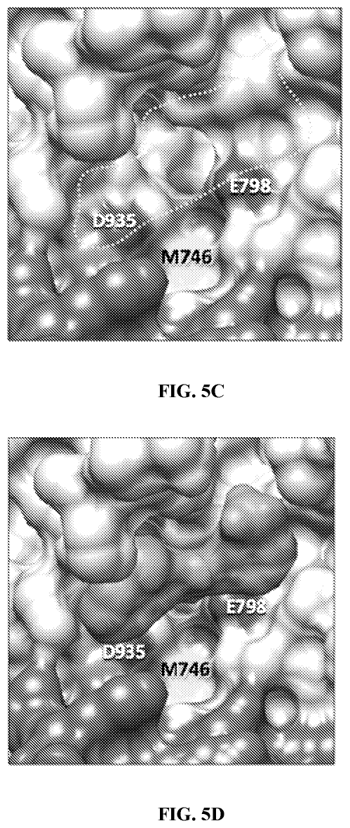

Potential ATP7A inhibitors were screened using an in silico approach. A model of ATP7A was generated based on its homology to a bacterial Cu-transporting P1B-type ATPase from Legionella pneumophila (LCopA) whose structure has been solved in a Cu-free E2 conformation (PDB structure 3RFU). The LCopA structure revealed a “platform” at the cytosolic membrane interface that is proposed to serve as a copper loading site for entry into the channel. As copper enters the mouth of the transporter, it is thought to be coordinated by a triad of Met-Glu-Asp residues that are found in all P1B-type ATPases (M746, E798 and D935 in ATP7A) (FIGS. 5A and 5B). Close inspection of the Cu-binding triad within the ATP7A model revealed a small pocket adjacent to the Cu-binding E798 and D935 residues that could potentially serve as a binding site for a small molecule inhibitor (FIG. 5C).

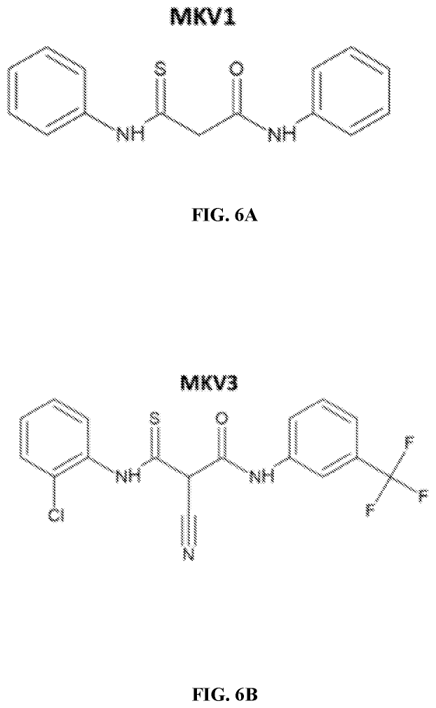

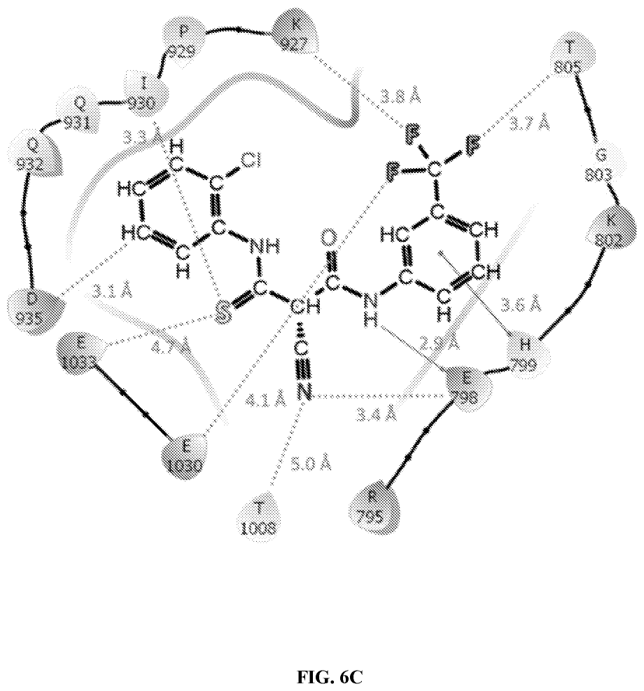

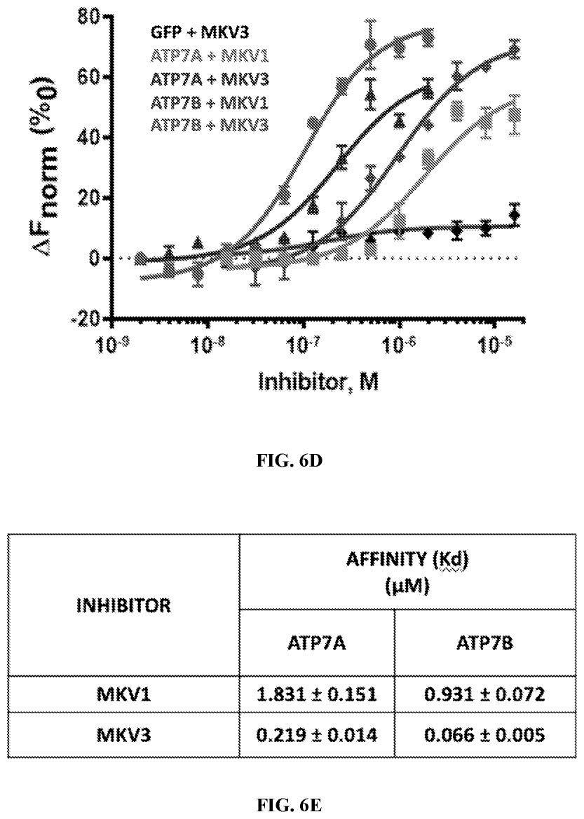

Using a computer-based approach, molecules predicted to bind within this pocket were screened. Out of over 8 million compounds screened, 500 of the best-fitting candidates were selected based on ‘Glide’ score, adherence to Lipinski's rule of five and visual inspection, and further analyzed using the ‘Induced Fit Docking (IFD)’ utility of the Schrödinger software suite. Ten compounds exhibiting the most favorable binding energy and Glide scores were synthesized and tested for their ability to bind and inhibit ATP7A. Two chemically related compounds, MKV1 and MKV3 (analogs of substituted N-phenyl-3-(phenylamino)-3-thioxopropanamide), were selected for further study. MKV3 is shown docked in the pocket of ATP7A in FIG. 5D and FIG. 6C.

MKV1 and MKV3 share a common molecular scaffold (FIGS. 6A and 6B). Microscale thermophoresis (MST) was used to determine if MKV1 and MKV3 can bind to ATP7A and ATP7B. Both MKV1 and MKV3 showed high affinity binding to both ATP7A-GFP and ATP7B-GFP but not to GFP alone (FIGS. 6D and 6E). Compared to MKV1, the affinity of MKV3 was approximately 10-fold higher for both ATP7A and ATP7B (FIG. 6E). Interestingly, both compounds bound ATP7B with higher affinity than ATP7A.

The computer-aided drug design suggested that three functional groups within MKV3 may contribute to its affinity, namely a chloro group (—Cl), a nitrile group (N) and a trifluoromethyl group (—CF3). Analyses of the MKV3-binding pocket in ATP7A reveals a deep crevice formed by residues from 4 helices (E1033, T1008, Q932 and E798) (FIG. 6C) which is surrounded by hydrophobic patches and hydrophilic residues. Docking studies with MKV3 reveal the nitrile group occupies the deep crevice with the m-(trifluoromethyl)phenylacetamide making hydrophobic contact with the patch, and the amide nitrogen is within hydrogen bonding distance of E798, which is thought to bind copper as it enters the transporter (FIG. 6C). The o-chlorophenyl occupies a shallow pocket formed by I930, Q931, Q932, and the predicted copper binding amino acid D935.

To evaluate the importance of these groups in MKV3, a second compound called MKV1 was generated. MKV1 was designed to lack the chloro, nitrile, and trifluoromethyl groups but retain the same core structure as MKV3 (FIG. 6A). MST was used to measure the binding affinity of MKV3 and MKV1 to both ATP7A and ATP7B. MST analysis of MKV3 revealed a binding affinity to ATP7A of 1.99×10⁻⁷ M (FIGS. 6D and 6E). The affinity of MKV3 for ATP7B was higher than for ATP7A at 0.76×10⁻⁷ M (FIGS. 6D and 6E). Compared to MKV3, the affinity of MKV1 was approximately 10-fold lower for both ATP7A and ATP7B, suggesting that the three functional groups on MKV3 make critical contacts with ATP7A (FIGS. 6D and 6E).

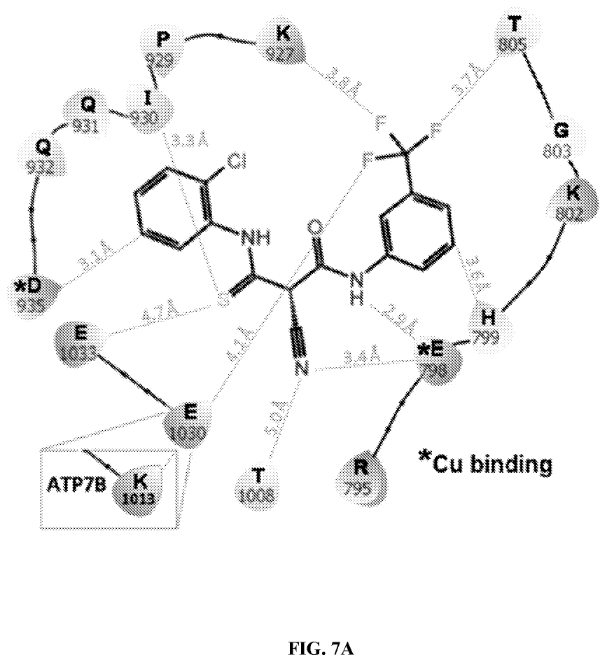

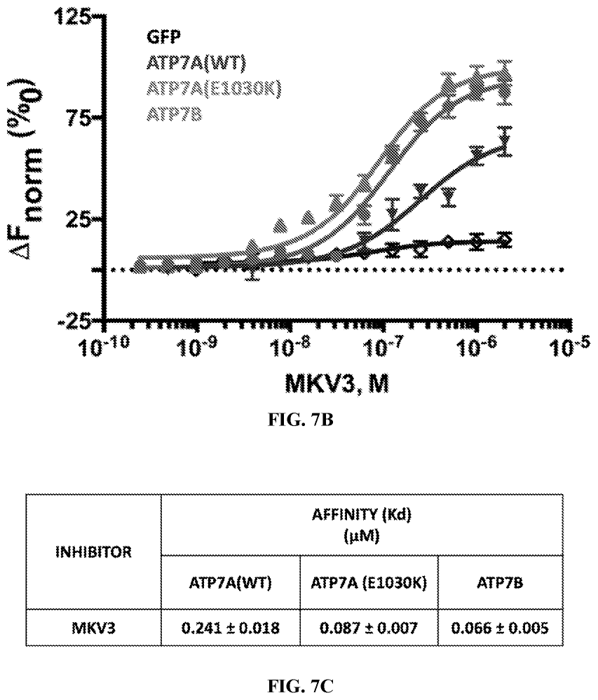

Analysis of the MKV binding pocket in ATP7A and ATP7B revealed a high degree of sequence conservation with only a single amino acid difference involving E1030 of ATP7A which corresponds to K1013 in ATP7B (FIG. 7A). The modeling predicts that E1030 and K1013 interact with a fluorine atom in MKV3 and that the stronger binding of fluorine to positively charged lysine relative to negatively charged glutamate may explain, in part, the higher affinity of MKV3 for ATP7B relative to ATP7A. This hypothesis was supported by the finding that an E1030K mutation in ATP7A increased the MKV3 binding affinity by approximately 2.7 fold (FIGS. 7B and 7C).

The chemical differences between MKV3 and MKV1 can be used to generate improved analogs using structure activity relationship (SAR) analyses.

### Example 5

### In Vitro and In Vivo Trials of MKV1 and MKV3

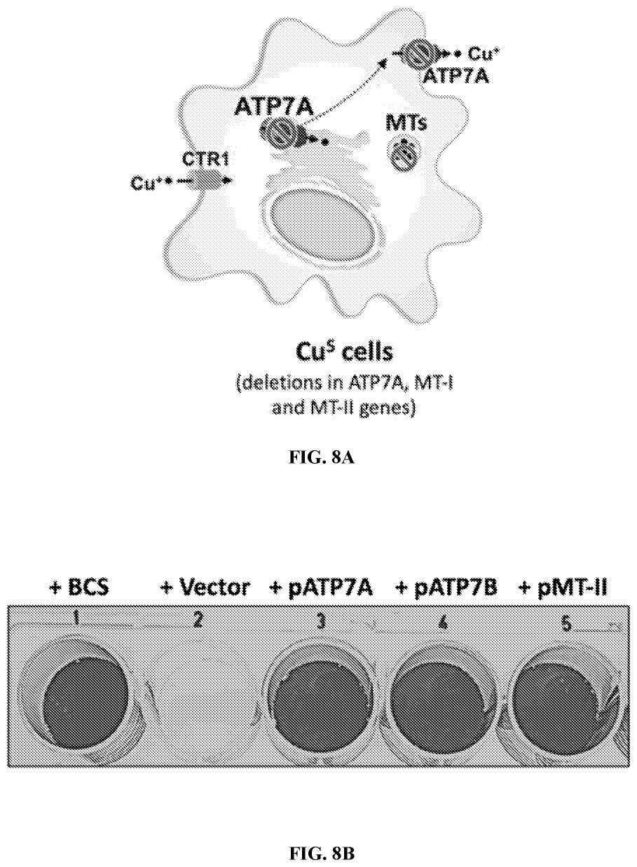

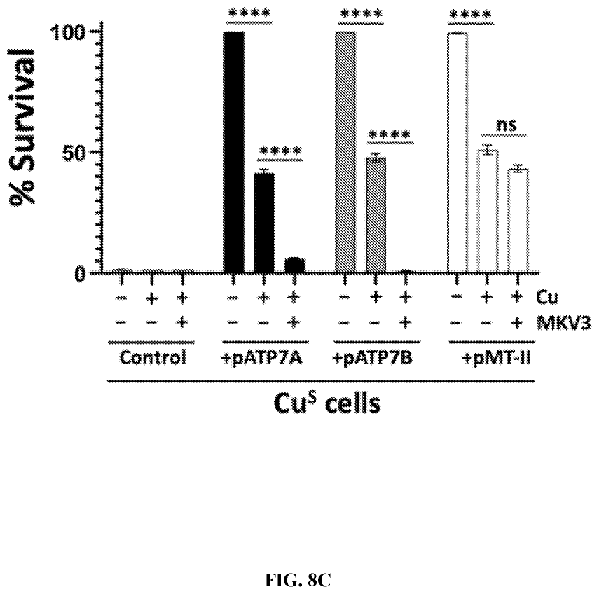

The ability of MKV3 to block ATP7A and ATP7B function in vitro was tested using the Cu^{S} cell line in which genes for ATP7A, MT-I and MT-II were deleted to create a highly copper sensitive cell line (FIG. 8A). Cu^{S} cell lines were derived that were complemented by either ATP7A, ATP7B or MT-II. It was found that MKV3 enhanced copper toxicity in the cells rescued with ATP7A and ATP7B, but not in the cells rescued with MT-II (FIGS. 8B and 8C). Thus, the ability of MKV3 to potentiate copper toxicity is dependent on the expression of ATP7A and ATP7B.

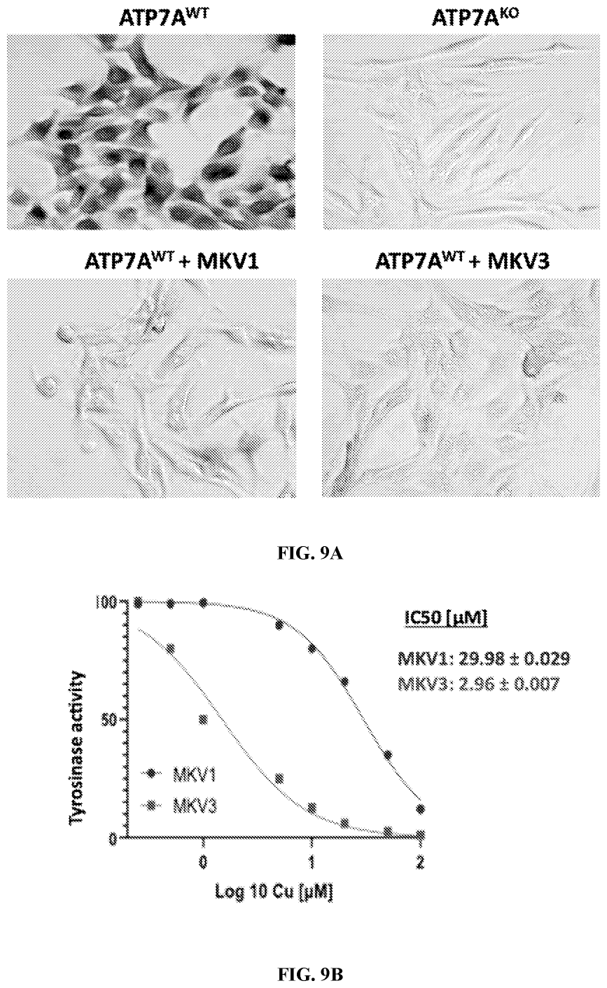

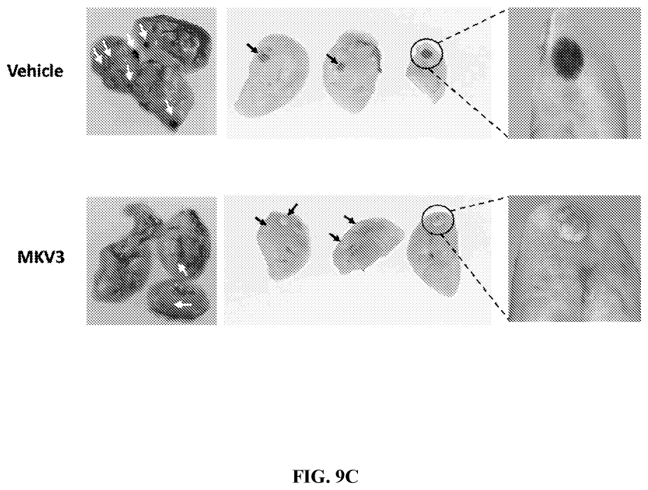

Other studies of ATP7A inhibition by MKV1 and MKV3 were performed using B16 melanoma cells. As a positive control for ATP7A inhibition, CRISPR-Cas9 technology was used to disrupt the ATP7A gene in B16 melanoma cells, which resulted in a loss of tyrosinase activity (ATP7A^{KO}) cells (FIG. 9A). Tyrosinase is a cuproenzyme involved in melanogenesis that receives copper via ATP7A in the secretory pathway. Consistent with the inhibition of ATP7A, treatment of B16-WT cells for 24 h with 5 μM MKV3 significantly reduced tyrosinase activity. Using B16 melanoma cells, we demonstrated that deletion of the ATP7A gene results in a loss of tyrosinase activity (FIG. 9A). Thus, reductions in tyrosinase activity in wild type B16 cells can be used as a proxy for inhibition of ATP7A.

Both MKV1 and MKV3 significantly reduced tyrosinase activity in B16-WT cells (FIGS. 9A and 9B). Consistent with the affinity data, MKV3 was more potent than MKV1. Dose-response studies demonstrated that MKV3 inhibits tyrosinase activity at a half-maximal inhibitory concentration (IC50) of 2.96 μM at 24 h. Importantly, MKV1 was also found to inhibit tyrosinase activity although this required significantly higher levels than MKV3 (FIG. 9B). This is consistent with lower affinity of MKV1 for ATP7A (FIG. 6E). In B16 cells, the half-maximal cytotoxic concentrations (CC50) were ˜75 μM for MKV3 and ˜270 μM for MKV1, as determined by MTT and crystal violet assays. In vivo studies demonstrated that systemic delivery of MKV3 inhibits tyrosinase activity in metastatic melanoma tumors in mice (FIG. 9C).

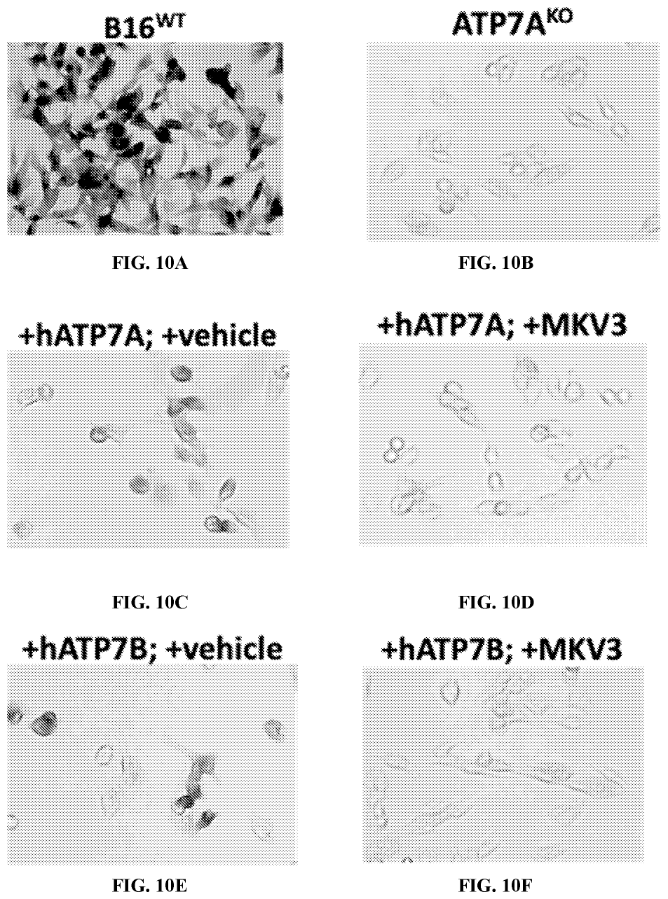

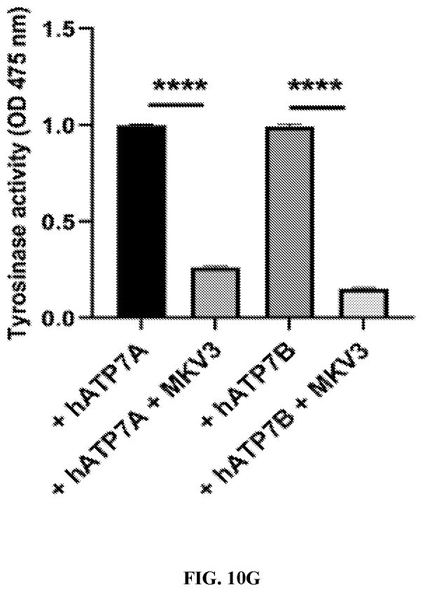

Using complementation studies in B16 ATP7A^{KO} cells it was shown that the activation of tyrosinase activity by expression of ATP7B is blocked by MKV3 (FIG. 10), thus providing evidence that MKV3 targets both ATP7A and ATP7B.

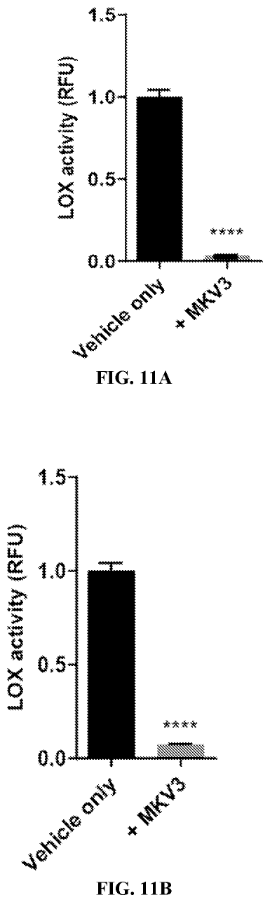

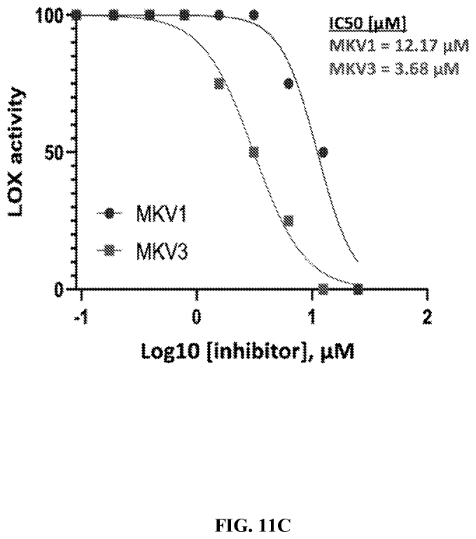

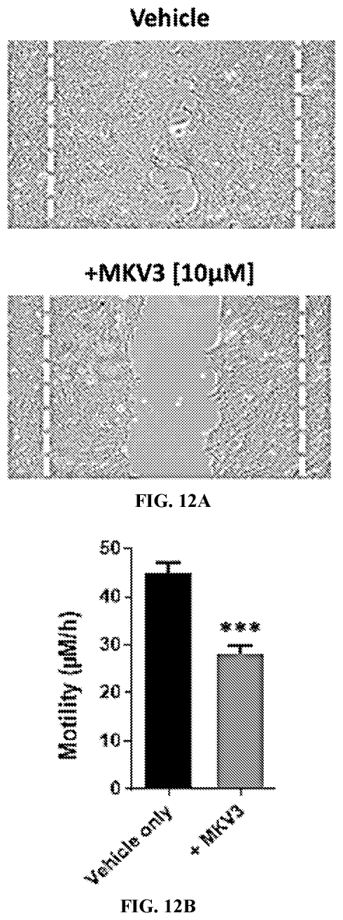

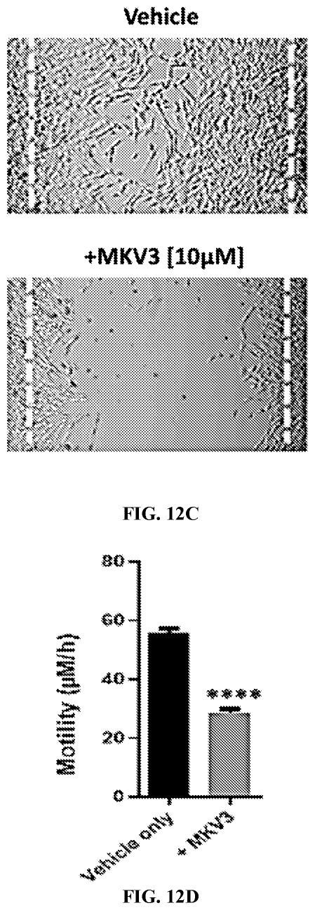

Using an independent assay of ATP7A activity, it was further found that MKV3 significantly reduced LOX activity in Lewis Lung Cancer (LLC) cells (FIG. 11A), 4T1 breast cancer cells (FIGS. 11B and 11C), and reduced gap closure in a scratch assay of cell motility (FIG. 12). As with tyrosinase, MKV1 also inhibited LOX activity but only at higher concentrations than for MKV3 (FIG. 11C).

In other studies, MKV3 was shown to block the motility of 4T1 breast cancer cells (FIGS. 12A and 12B) as well as LLC lung cancer cells (FIGS. 12C and 12D). Since previous studies have shown that LOX activity is important for cell motility, the inhibitory effects of MKV3 on cell motility are consistent with LOX inhibition.

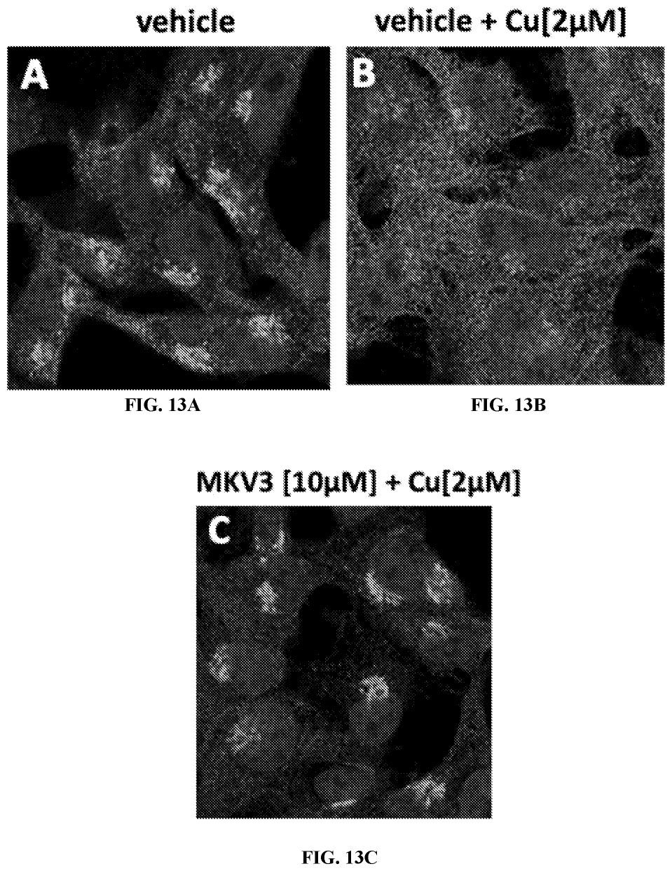

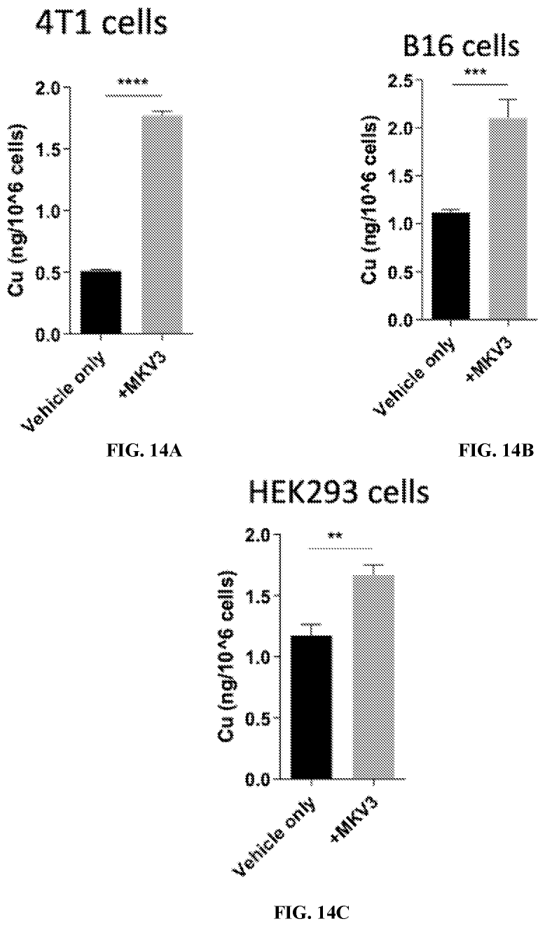

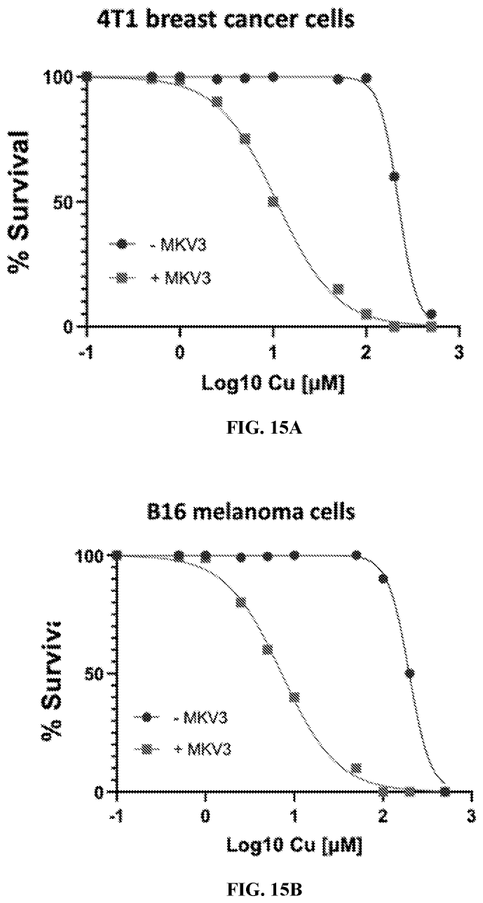

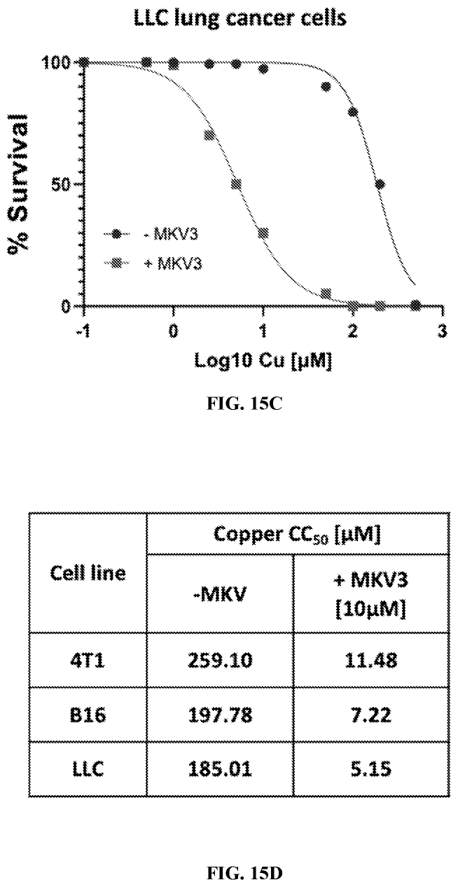

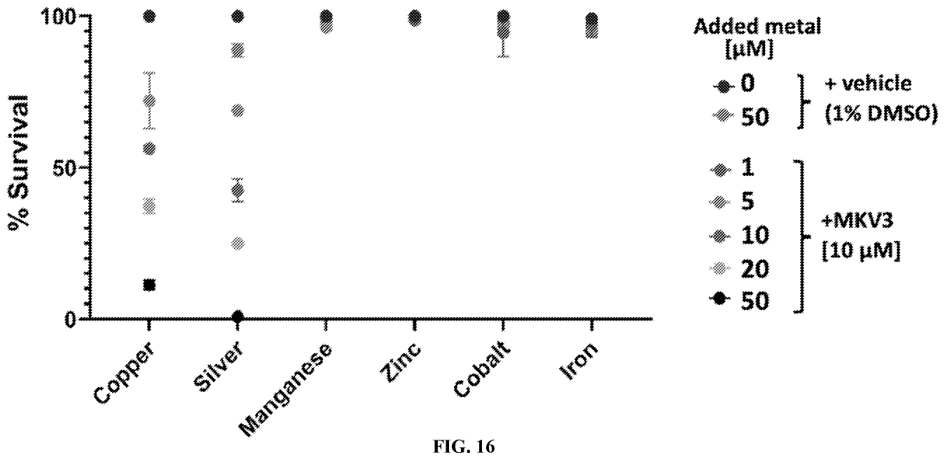

In other studies, MKV3 was found to block the copper-stimulated trafficking of ATP7A from the perinuclear region to cytoplasmic vesicles in 4T1 breast cancer cells (FIG. 13). MKV3 treatment also resulted in hyperaccumulation of copper in 4T1 (FIG. 14A), B16 (FIG. 14B) and HEK293 cells (FIG. 14C). Consistent with inhibition of copper export, MKV3 significantly increased the sensitivity of 4T1 (FIG. 15A), B16 (FIG. 15B) and LLC cells (FIG. 15C) to copper added to the medium (FIG. 15D). Since copper-stimulated trafficking and copper export functions of ATP7A are dependent on its catalytic activity, these data provide additional evidence that MKV3 is an inhibitor of ATP7A. Additionally, MKV3 enhanced sensitivity to silver, but not to other heavy metals (FIG. 16). As silver is a known substrate of copper transporting P1B-type ATPases, these data provide further evidence supporting MKV3 as an inhibitor of this class of proteins.

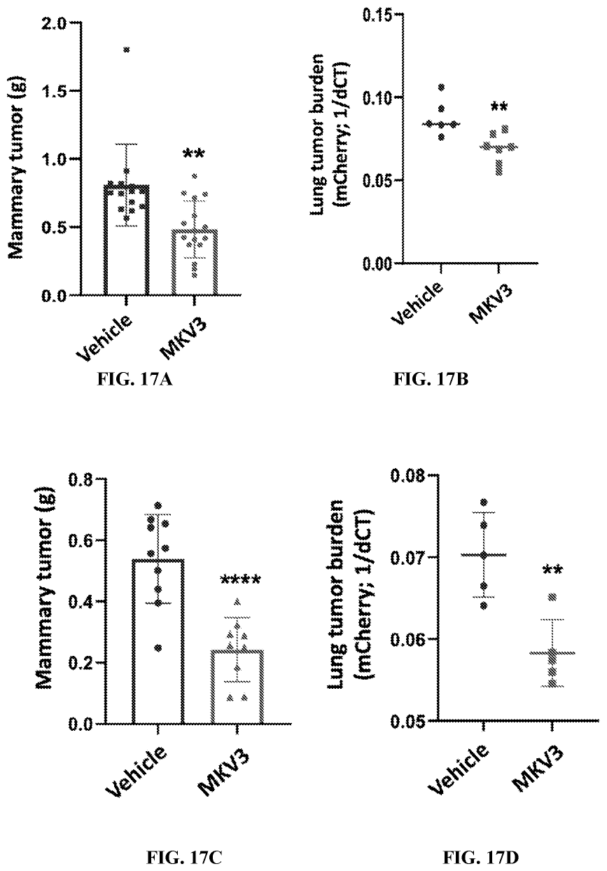

Pilot studies were performed to test whether MKV3 blocks ATP7A-mediated tumor growth and metastasis in mice using the orthotopic 4T1 model of breast cancer. Using two different modes of administration (subcutaneous and intravenous), MKV3 significantly reduced tumor weight and metastatic burden compared to vehicle control (FIG. 17A-17D).

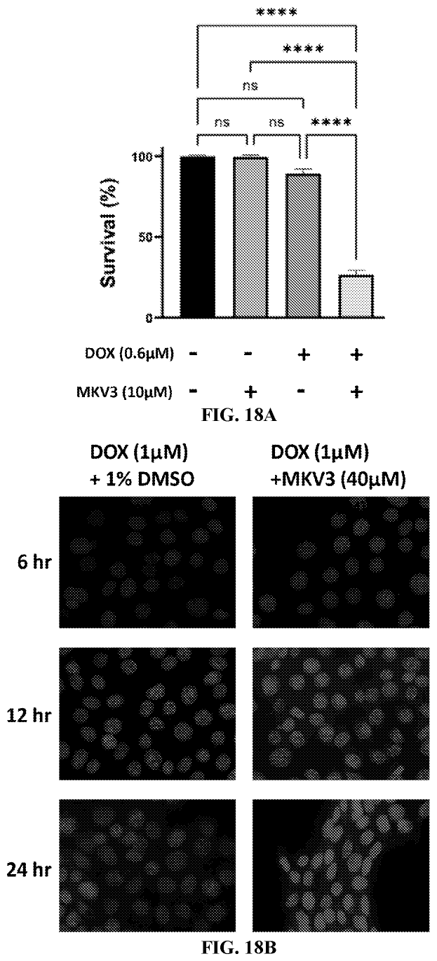

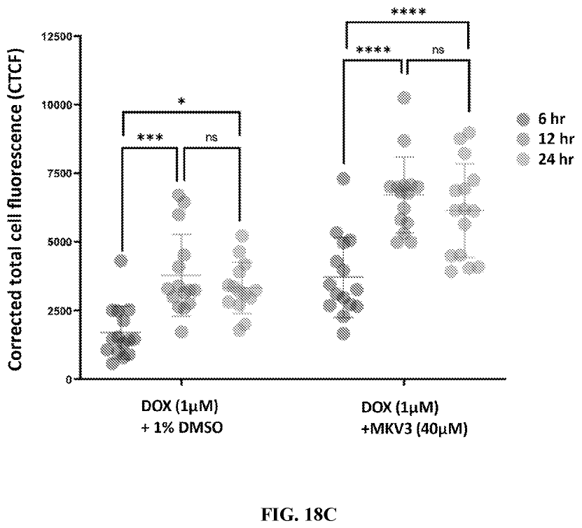

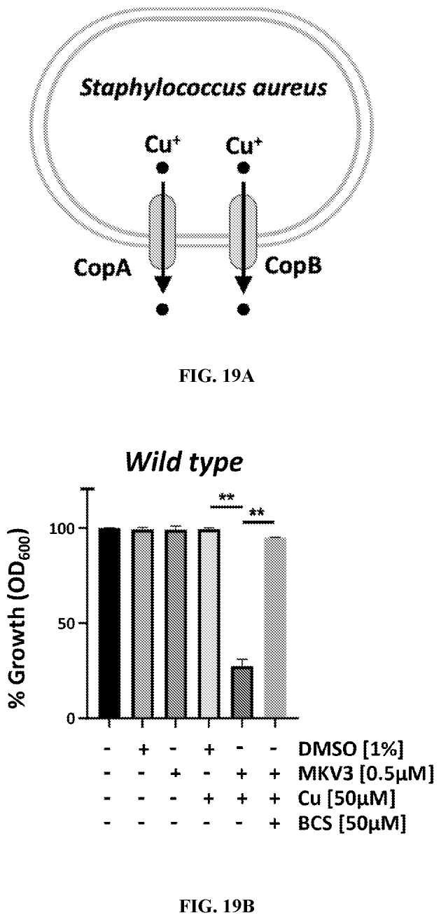

Previous studies have shown that ATP7A and ATP7B are multidrug resistance proteins that confer tolerance to multiple types of anti-cancer chemotherapy drugs including cisplatin and doxorubicin (DOX). Taking advantage of the auto-fluorescent properties of DOX, it was found that MKV3 significantly reduced the accumulation DOX in 4T1 breast cancer cells (FIGS. 18B and 18C), and significantly increased the cytotoxicity of this chemotherapy agent (FIG. 18A). Taken together, the in vitro and in vivo studies indicate that MKV3 is a novel high-affinity inhibitor of ATP7A.

### Example 6

MKV3 Inhibition of Methicillin-Resistant Staphylococcus aureus

MKV3 was also found to inhibit the growth of wild type methicillin-resistant Staphylococcus aureus (MRSA) in a Cu-dependent manner, indicating potential use of MKV3 analogs as novel antibiotics.

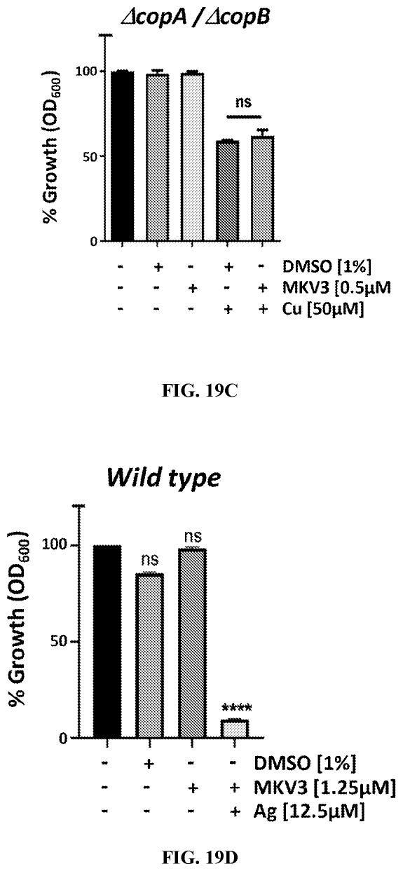

Wild type MRSA expresses two copper-exporting P1B-type ATPases, CopA and CopB that are required for copper tolerance (FIG. 19A). The growth of wild type MRSA was not affected by either MKV3 (0.5 μM) or copper (50 μM) added separately to the culture medium (FIG. 19B). However, both MKV3 and copper added together to the culture medium significantly inhibited growth of wild type MRSA, which could be prevented by addition of a copper chelator BCS (FIG. 19B). The same experiment was performed using MRSA in which both CopA and CopB genes were deleted (ΔcopA/ΔcopB). Consistent with the known role of CopA and CopB in copper tolerance, the growth of the ΔcopA/ΔcopB strain was inhibited by approximately 50% when copper alone (50 μM) was added to the culture medium (FIG. 19C). However, unlike the wild type MRSA, MKV3 did not increase copper sensitivity in the ΔcopA/ΔcopB strain (FIG. 19C), demonstrating that MKV3-mediated potentiation of copper toxicity is dependent on the presence of CopA and CopB proteins. Additionally, it was found that MKV3 increased the sensitivity of wild type MRSA to silver (FIG. 19D). Taken together, these data demonstrate that MKV3 is an inhibitor of evolutionarily distant P1B-type ATPases spanning prokaryotes and eukaryotes.

### Example 7

### Structure Activity Relationship (SAR) Analysis of Structural Analogs of MKV1 and MKV3

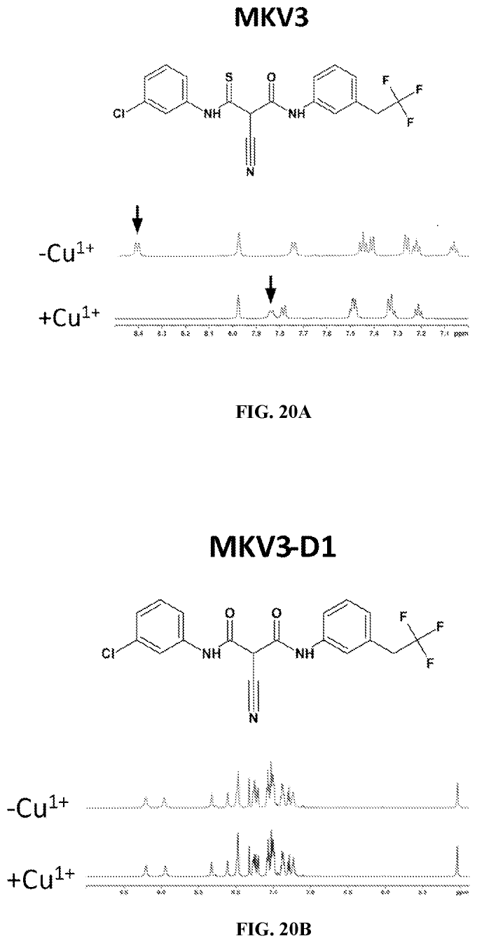

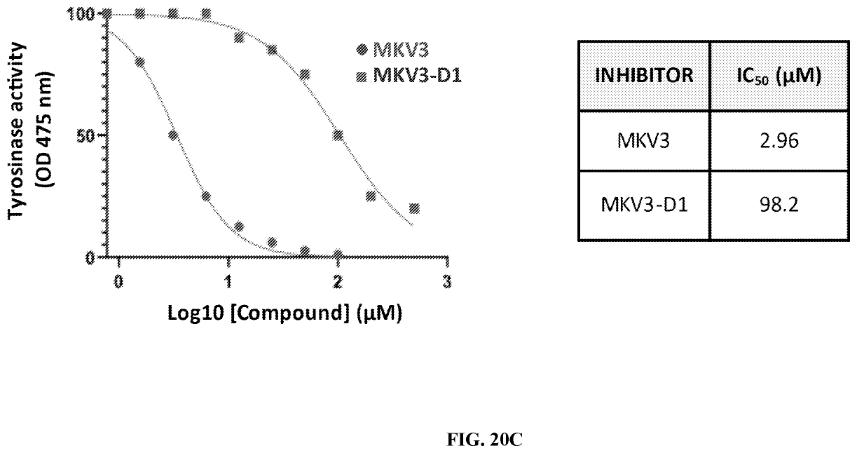

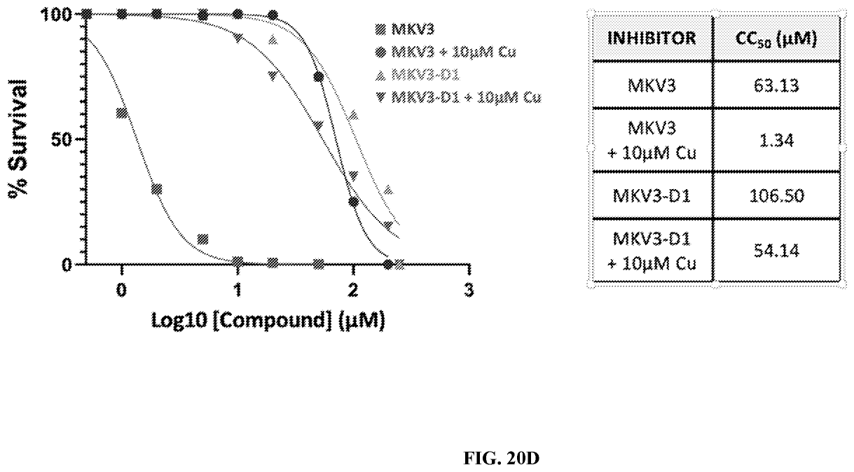

Initial structural analysis focused on the importance of the sulfur in MKV3. The predicted MKV3 binding pocket is located in close proximity to the copper-binding triad in P1B-type ATPases. It was thought that MKV3 may provide a coordinating Cu(I) ligand as a mechanism of preventing copper movement into the channel domain. To test this hypothesis, a control compound was generated in which the sulfur atom in the thioamide was replaced with oxygen to generate a corresponding amide (MKV3-D1). FIGS. 20A and 20B show the chemical structures of MKV3 and MKV3-D1 as well as the results of NMR experiments using MKV3 and MKV3-D1 in the presence and the absence of CuI in 9:1 d6-DMSO/D20. The results show that MKV3 signals change upon addition of CuI while no such changes were observed with MKV3-D1. To test whether the thioamide in MKV3 is essential for inhibition of ATP7A copper transport activity, the ability of MKV3-D1 to inhibit tyrosinase activity was tested in B16 melanoma cells. FIG. 20C shows the relationship between tyrosinase activity and compound concentration. The data show that MKV3-D1 has a far higher tyrosinase 50% inhibitory (IC₅₀) concentration than MKV3. Using another assay of ATP7A inhibition, the ability of MKV3-D1 to sensitize 4T1 breast cancer cells to copper was tested. FIG. 20D compares the concentrations of MKV3 and MKV3-D1 needed to reduce survival of 4T1 cells in the presence or absence of 10 μM copper. The data show that 10 μM copper resulted in a 47-fold reduction in the MKV3 concentration required to kill 50% of 4T1 cells (CC₅₀) (63.13 μM vs 1.34 μM). By contrast, 10 μM copper reduced the CC₅₀ of MKV3-D1 by only 5.6 fold (106.50 vs 54.14). These data indicate that the thioamide in MKV3 is required for both copper binding and inhibitory activity.

Table 1 lists the names and chemical structures of MKV3 derivative compounds, the 50% inhibitory concentration (IC₅₀) of each compound for tyrosinase activity in B16 cells, and the affinity of each compound for ATP7A using MST analysis. The results demonstrate that the chlorine, nitrile and trifluoromethyl groups of MKV3 are important for inhibitory activity and binding to ATP7A.

TABLE 1

| | | | ATP7A |
|---|---|---|---|
| | | Tyrosinase | Binding |
| | | Inhibition | Affinity |
| | Compound Structure | [IC50;μM] | K_{D}(nM) |
| MKV1 | 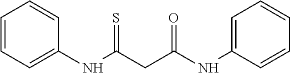 [SMILES: O=C(CC(=S)Nc1ccccc1)Nc1ccccc1] | 29.98 ± 0.029 | 1831 ± 151 |
| MKV1A | 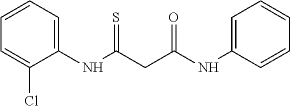 [SMILES: O=C(CC(=S)Nc1ccccc1Cl)Nc1ccccc1] | 21.559 ± 0.005 | 363 ± 24 |
| MKV1B | 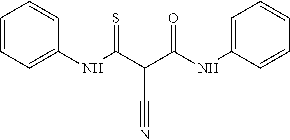 [SMILES: N#CC(C(=O)Nc1ccccc1)C(=S)Nc1ccccc1] | 21.871 ± 0.129 | 354 ± 21 |
| MKV1C | 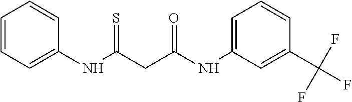 [SMILES: O=C(CC(=S)Nc1ccccc1)Nc1cccc(C(F)(F)F)c1] | 16.185 ± 0.023 | 430 ± 16 |
| MKV1D | 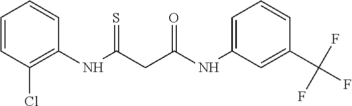 [SMILES: O=C(CC(=S)Nc1ccccc1Cl)Nc1cccc(C(F)(F)F)c1] | 12.222 ± 0.005 | 708 ± 42 |
| MKV1E | 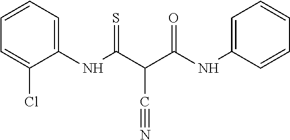 [SMILES: N#CC(C(=O)Nc1ccccc1)C(=S)Nc1ccccc1Cl] | 11.700 ± 0.001 | 1061 ± 54 |
| MKV1F | 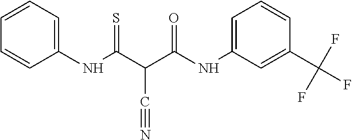 [SMILES: N#CC(C(=O)Nc1cccc(C(F)(F)F)c1)C(=S)Nc1ccccc1] | 8.719 ± 0.014 | 1000 ± 59 |
| MKV3 | 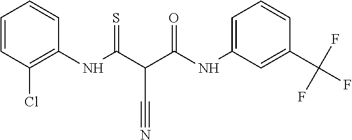 [SMILES: N#CC(C(=O)Nc1cccc(C(F)(F)F)c1)C(=S)Nc1ccccc1Cl] | 2.96 ± 0.007 | 219 ± 14 |

It is to be understood that while the invention has been described in conjunction with the detailed description thereof, the foregoing description is intended to illustrate, and not limit the scope of the invention, which is defined by the scope of the appended claims. Other embodiments, advantages, and modifications are within the scope of the following claims. Any reference to accompanying drawings which form a part hereof, are shown, by way of illustration only. It is understood that other embodiments may be utilized and structural changes may be made without departing from the scope of the present disclosure. All publications discussed and/or referenced herein are incorporated herein in their entirety.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

1. A compound having one of the following formulae: 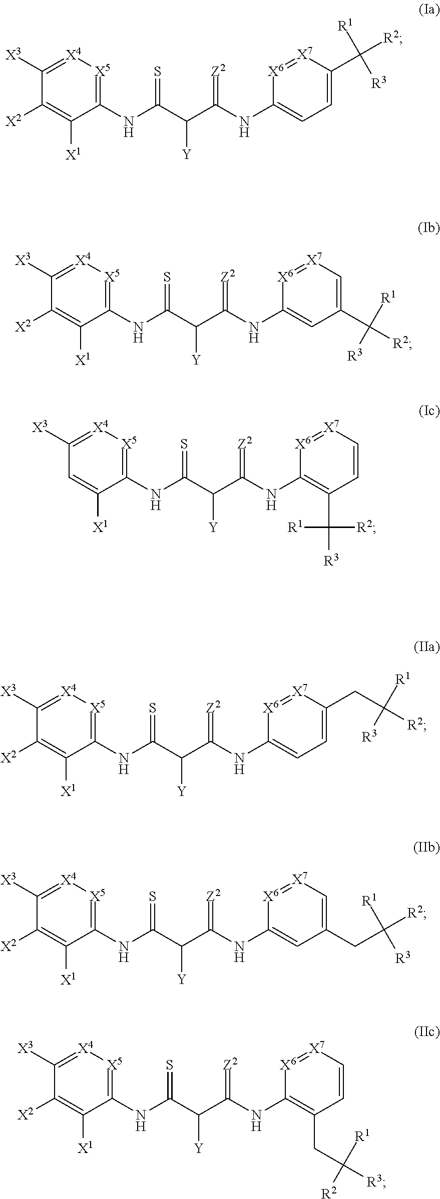 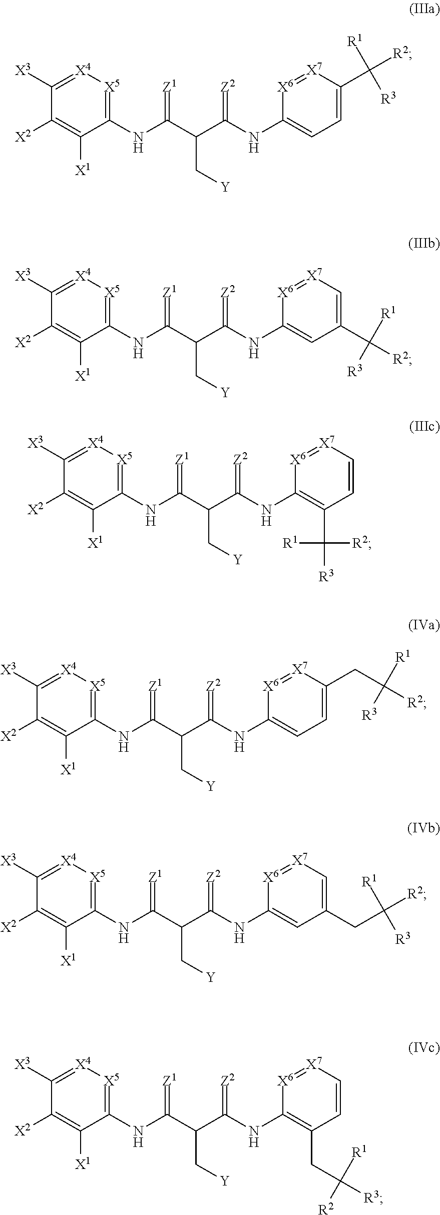 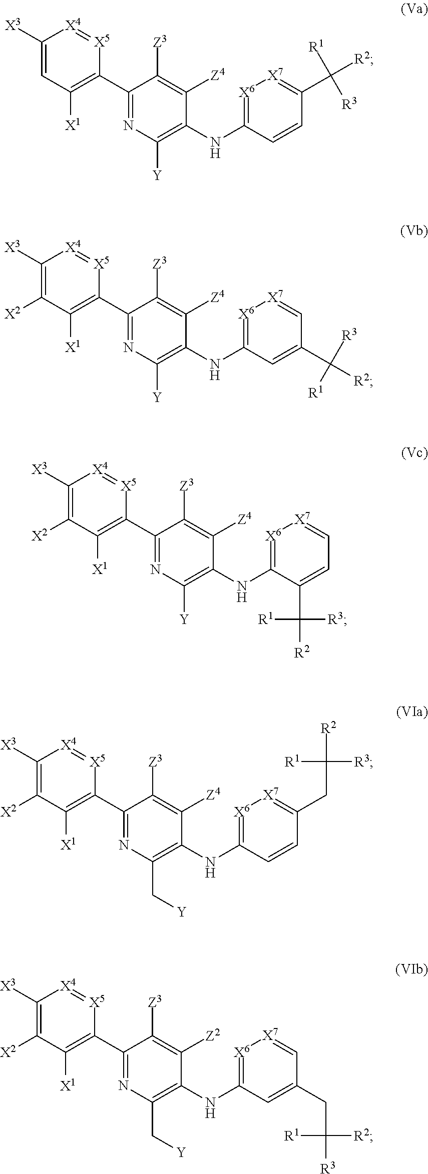 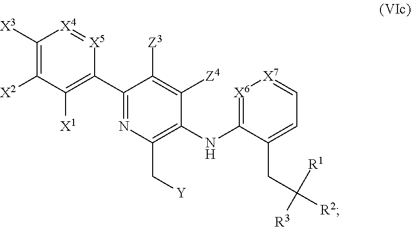 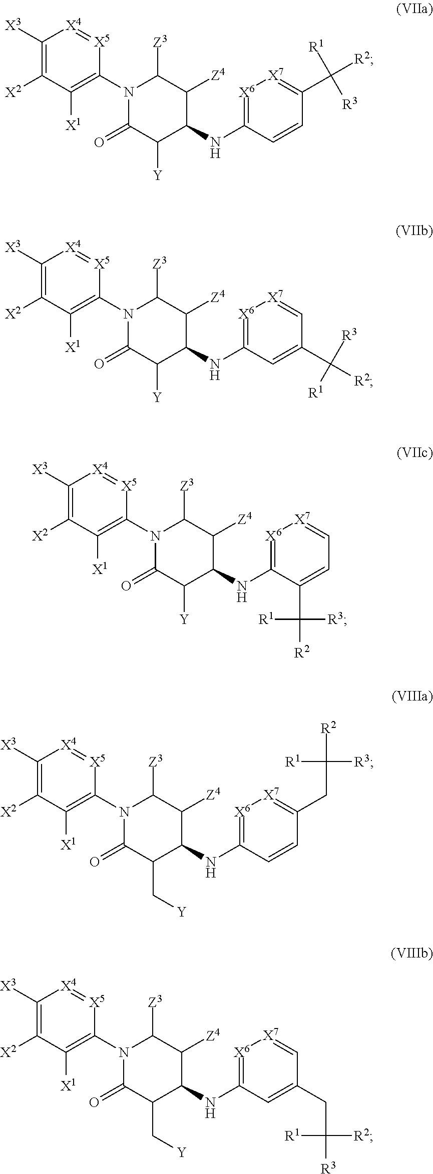 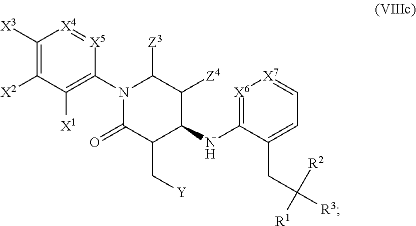
a resonance structure thereof, or
a pharmaceutically acceptable salt or aqueous solution or dispersion thereof,
wherein each of R¹, R², and R³ individually are a halogen, hydrogen, hydroxyl, amine (NH₂), 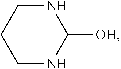
or absent;
wherein X¹, X², and X³ individually are a hydrogen, halogen, O, or C═O;
wherein X⁴, X³, X⁶, and X⁷ individually are C or N;
wherein at least two of X¹, X², and X³ are hydrogen;
wherein Y is HC≡N, CH₃, CH₃—CH₄, C═O, hydrogen, or absent; and
wherein Z¹, Z², Z³, and Z⁴ individually are S, O, amine (NH₂), —SH, —OH, —H, ═S, ═O, =amine (═NH₂), or a protonated S, O, amine (NH₂).

2. The compound of claim 1, wherein the compound of Formula Ia, Ib, or Ic is one of the following formulae: 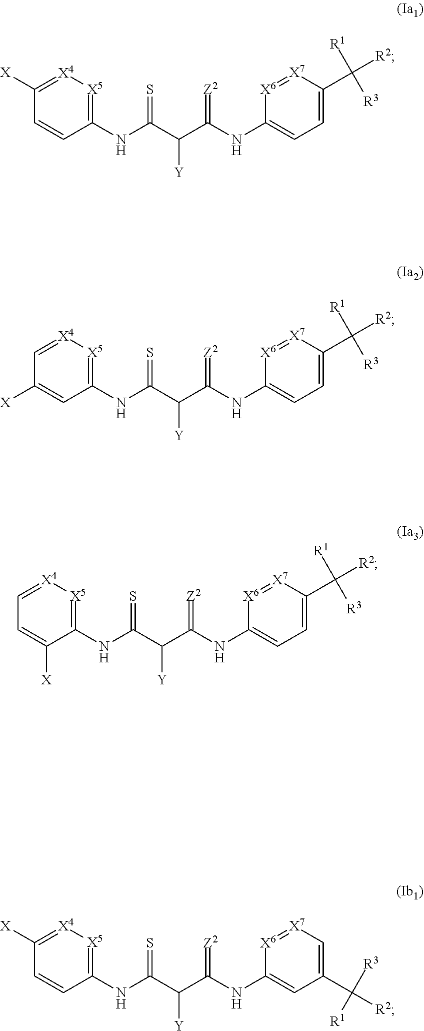 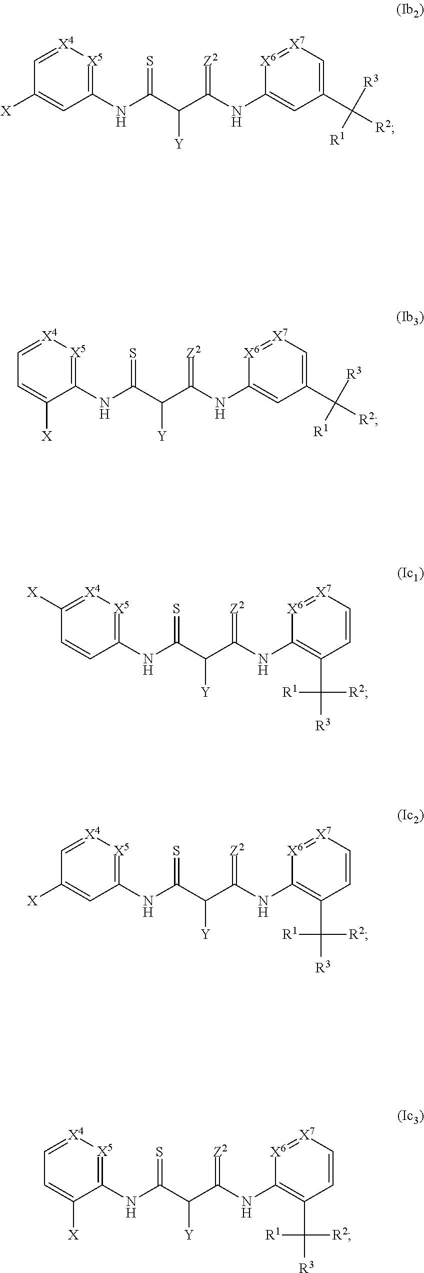
a resonance structure thereof, or
a pharmaceutically acceptable salt or aqueous solution or dispersion thereof,
wherein each of R¹, R², and R³ individually are a halogen, hydrogen, hydroxyl, amine (NH₂), 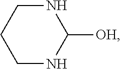
or absent;
wherein X is a hydrogen, halogen, O, or C═O;
wherein X⁴, X³, X⁶, and X⁷ individually are C or N;
wherein Y is HC≡N, CH₃, CH₃—CH₄, C═O, hydrogen, or absent; and
wherein Z² is S, O, amine (NH₂), —SH, —OH, or —H.

3. The compound of claim 1, wherein the compound of Formula IIa, IIb, or IIc is one of the following formulae: 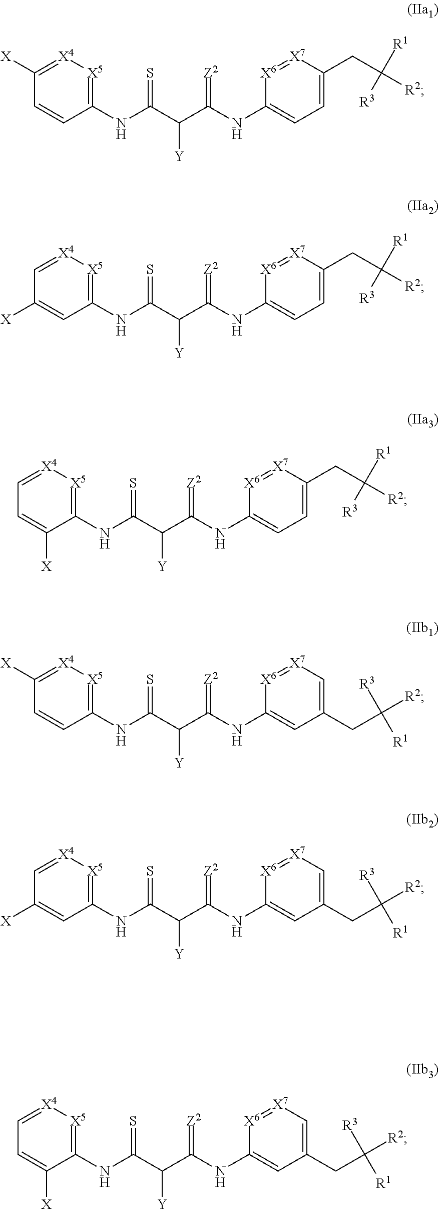 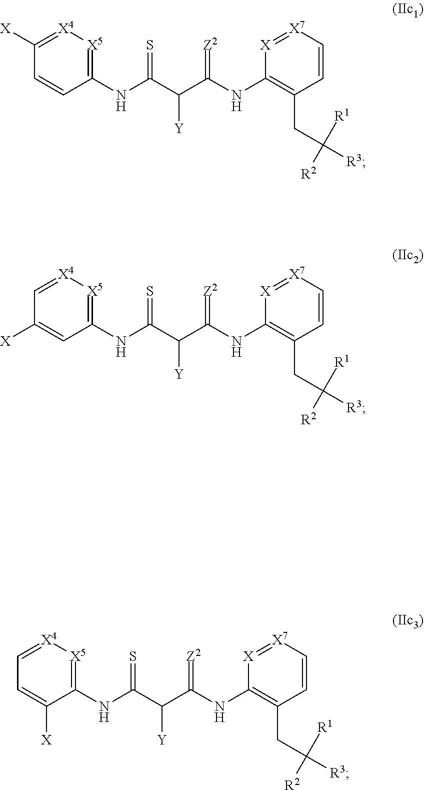
a resonance structure thereof, or
a pharmaceutically acceptable salt or aqueous solution or dispersion thereof,
wherein each of R¹, R², and R³ individually are a halogen, hydrogen, hydroxyl, amine (NH₂), 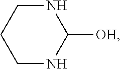
or absent;
wherein X is a hydrogen, halogen, O, or C═O;
wherein X⁴, X⁵, X⁶, and X⁷ individually are C or N;
wherein Y is HC≡N, CH₃, CH₃—CH₄, C═O, hydrogen, absent; and
wherein Z² is S, O, amine (NH₂), —SH, —OH, or —H.

4. The compound of claim 1, wherein the compound of Formula IIIa, IIIb, IIIc, IVa, IVb, or IVc is one of the following formulae: 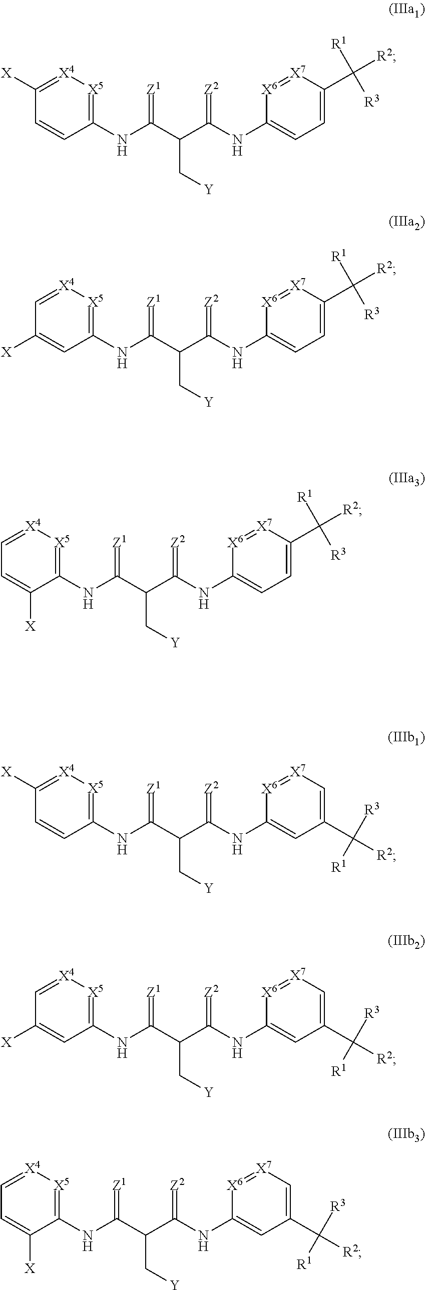 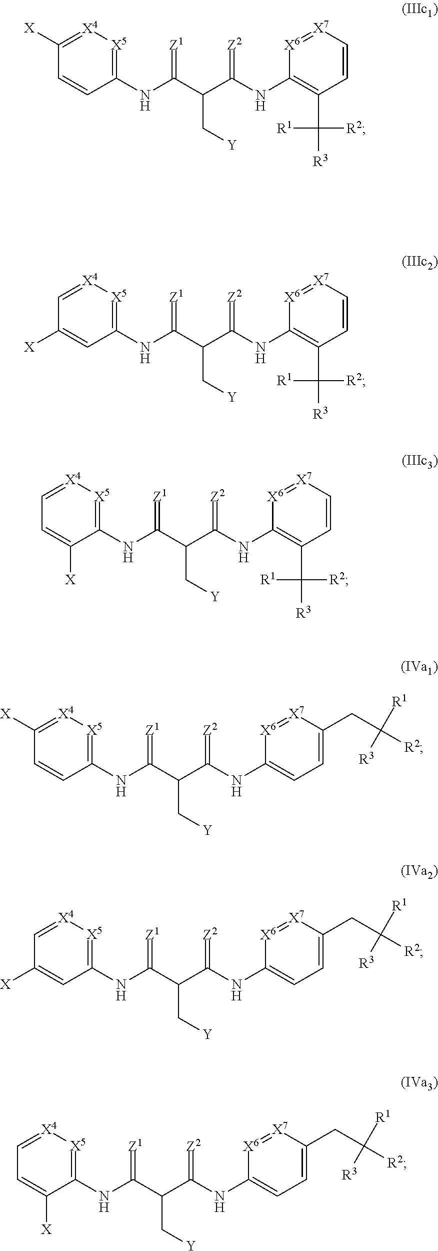 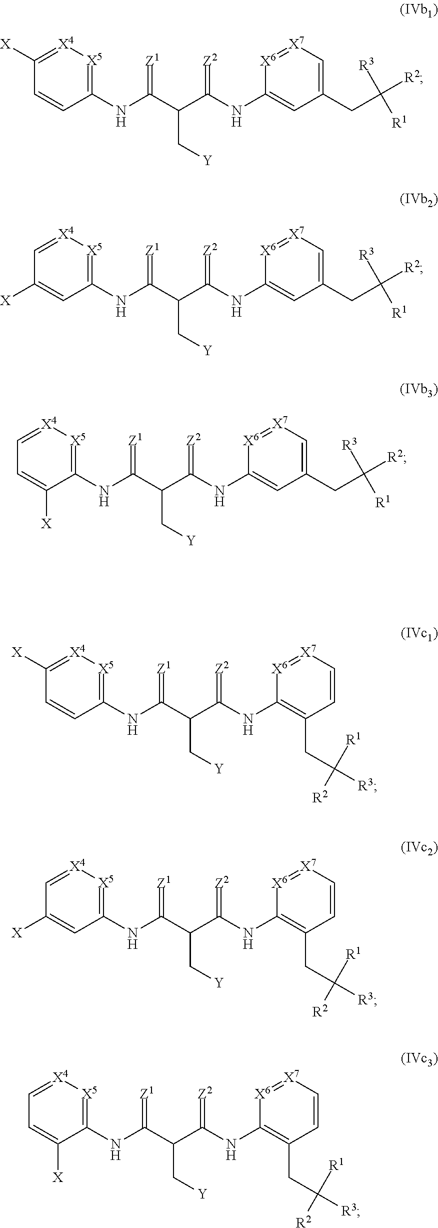
a resonance structure thereof, or
a pharmaceutically acceptable salt or aqueous solution or dispersion thereof,
wherein each of R¹, R², and R³ individually are a halogen, hydrogen, hydroxyl, amine (NH₂), 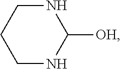
or absent;
wherein X is a halogen, O, or C═O;
wherein X⁴, X³, X⁶, and X⁷ individually are C or N;
wherein Y is HC≡N, CH₃, CH₃—CH₄, C═O, hydrogen, or absent; and
wherein Z¹ and Z² individually are S, O, amine (NH₂), —SH, —OH, or —H.

5. The compound of claim 1, wherein the compound of Formula Va, Vb, Vc, VIa, VIb, VIc, VIIa, VIIb, VIIc, VIIIa, VIIIb, or VIIIc is one of the following formulae 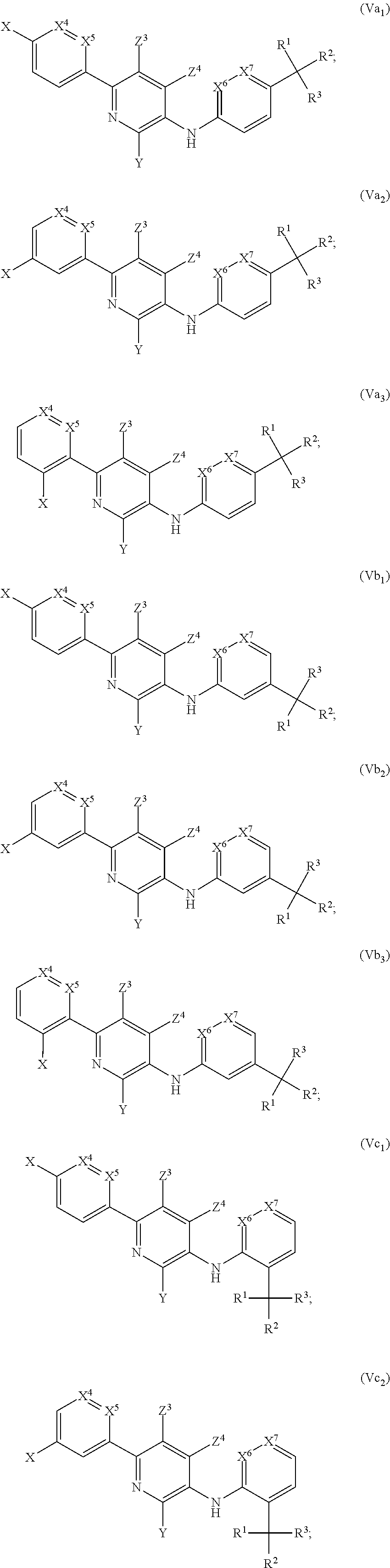 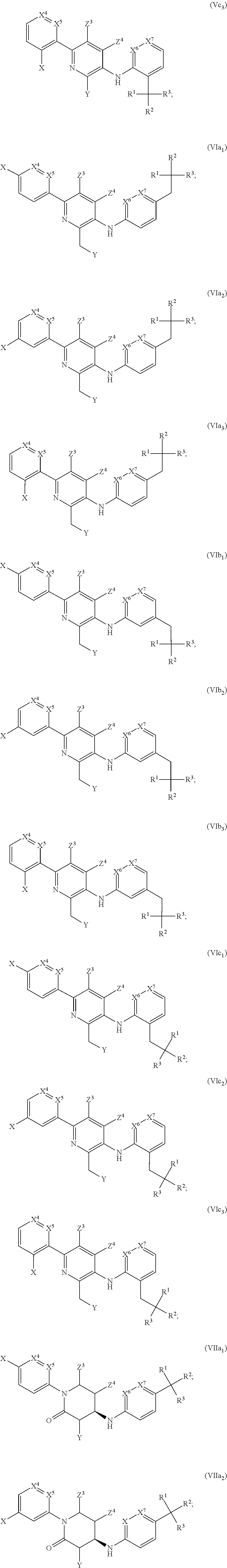 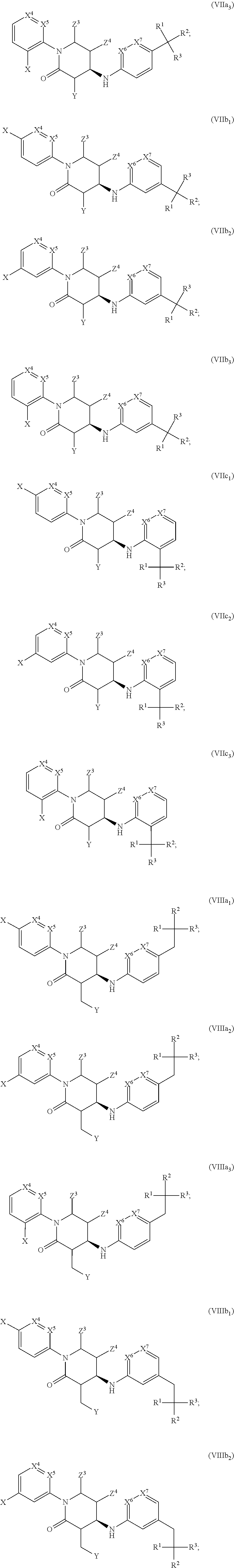 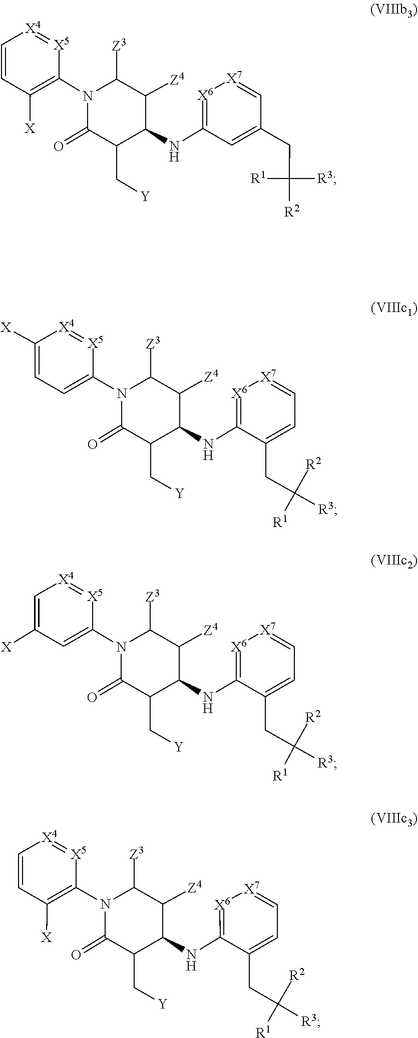
a resonance structure thereof, or
a pharmaceutically acceptable salt or aqueous solution or dispersion thereof,
wherein each of R¹, R², and R³ individually are a halogen, hydrogen, hydroxyl, amine (NH₂), 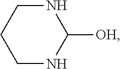
or absent;
wherein X is a halogen, O, or C═O;
wherein X⁴, X⁵, X⁶, and X⁷ individually are C or N;
wherein Y is HC≡N, CH₃, CH₃—CH₄, C═O, hydrogen, or absent; and
wherein Z³ and Z⁴ individually are S, O, amine (NH₂), ═S, ═O, =amine (═NH₂), —SH, —OH, —H, or a protonated S, O, amine (NH₂).

6. The compound of claim 1, wherein the compound is any one of Formulae Ia, Ib, Ic, IIa, IIb, IIc, IIIa, IIIb, IIIc, IVa, IVb, IVc, Va, Vb, Vc, VIa, VIb, VIc, VIIa, VIIb, VIIc, VIIIa, VIIIb, VIIIc, or a resonance structure thereof, wherein:
i. R¹ is a hydroxyl and R² and R³ are each F, Br, Cl, or I, wherein X¹, X², and X³ is hydrogen, Cl, Br, F, I, O, or C═O, wherein X⁴, X³, X⁶, and X⁷ individually are C or N, and wherein Y is HC≡N, CH₃, CH₃—CH₄, C═O, hydrogen or absent;
ii. R³ is a hydroxyl and R¹ and R² are each F, Br, Cl, or I, wherein X¹, X², and X³ is hydrogen, Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iii. each of R¹, R², and R³ is independently F, Br, Cl, I, hydrogen, or absent, wherein X¹, X², and X³ is independently hydrogen, Cl, Br, F, I, O, or C—O, wherein X⁴, X⁵, X⁶, and X⁷ individually are C or N, and wherein Y is HC≡N, CH₃, CH₃—CH₄, C═O, hydrogen or absent;
iv. R² is 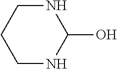
and R¹ and R³ are each F, Br, Cl, or I, wherein X¹, X², X³ is hydrogen, Cl, Br, F, I, O, or C═O, wherein X⁴, X⁵, X⁶, and X⁷ individually are C or N, wherein Y is HC≡N, CH₃, CH₃—CH₄, C═O, hydrogen or absent; or
v. R¹ or R³ is —NH₂, wherein X¹, X², and X³ is hydrogen, Cl, Br, F, I, O, or C—O, wherein X⁴, X⁵, X⁶, and X⁷ individually are C or N, and wherein Y is HC≡N, CH₃, CH₃—CH₄, C═O, hydrogen or absent.

7. The compound of claim 2, wherein the compound is any one of Formulae Ia₍₁₋₃₎, Ib₍₁₋₃₎, or Ic₍₁₋₃₎, wherein:
i. R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N CH₃, CH₃—CH₄, C═O, hydrogen or absent;
ii. R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N CH₃, CH₃—CH₄, C═O, hydrogen or absent;
iii. each of R¹, R², and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iv. R² is 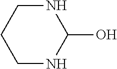
and R¹ and R³ are each F, Br, Cl, or I, wherein X is Cl, Br, F, I, O, or C═O, wherein X⁴, X⁵, X⁶, and X⁷ individually are C or N, and wherein Y is HC≡N CH₃, CH₃—CH₄, C═O, hydrogen or absent; or
v. R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

8. The compound of claim 3, wherein the compound is any one of Formulae IIa₍₁₋₃₎, IIb₍₁₋₃₎ or IIc₍₁₋₃₎ wherein:
i. R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N CH₃, CH₃—CH₄, C═O, hydrogen or absent;
ii. R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N CH₃, CH₃—CH₄, C═O, hydrogen or absent;
iii. each of R¹, R², and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iv. R² is 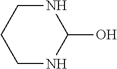
and R¹ and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; or
v. R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

9. The compound of claim 4, wherein the compound is any one of Formulae IIIa₍₁₋₃₎, IIIb₍₁₋₃₎, IIIc₍₁₋₃₎, IVa₍₁₋₃₎, IVb₍₁₋₃₎, IVc₍₁₋₃₎ wherein:
i. R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
ii. R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iii. each of R¹, R² and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iv. R² is 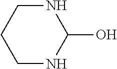
and R¹ and R³ are each F, Br, Cl, or I, wherein X is Cl, Br, F, I, O, or C═O, wherein X⁴, X³, X⁶, and X⁷ individually are C or N, and wherein Y is HC≡N, CH₃, CH₃—CH₄, C═O, hydrogen or absent; or
v. R¹ or R³ is —NH₂, wherein X is Cl, Br, F, I, O, or C—O, wherein X⁴, X⁵, X⁶, and X⁷ individually are C or N, and wherein Y is HC≡N, CH₃, CH₃—CH₄, C═O, hydrogen or absent.

10. The compound of claim 5, wherein the compound is any one of Formulae Va₍₁₋₃₎, Vb₍₁₋₃₎, Vc₍₁₋₃₎, VIa₍₁₋₃₎, VIb₍₁₋₃₎, VIC₍₁₋₃₎, VIIa₍₁₋₃₎, VIIb₍₁₋₃₎, VIIc₍₁₋₃₎, VIIIa₍₁₋₃₎, VIIIb₍₁₋₃₎, VIIIc₍₁₋₃₎, wherein:
i. R¹ is a hydroxyl and R² and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
ii. R³ is a hydroxyl and R¹ and R² are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iii. each of R¹, R² and R³ is F, Br, Cl, I, hydrogen, or absent, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent;
iv. R² is 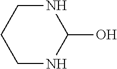
and R¹ and R³ are each F, Br, Cl, or I, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent; or
v. R¹ or R³ is —NH₂, wherein X is Cl, Br, F, I, O, or C═O, wherein X⁴, X⁵, X⁶, and X⁷ individually are C or N, and wherein Y is HC≡N, CH₃, CH₃—CH₄, C═O, hydrogen or absent.

11. A pharmaceutical composition, comprising:
the compound of claim 1; and a pharmaceutically acceptable carrier.

12. A method for preventing and/or treating a health condition and/or disease in a subject in need thereof, comprising:
administering to the subject a therapy comprising a therapeutically effective amount of the compound according to claim 1 or a pharmaceutical composition comprising the compound and a pharmaceutically acceptable carrier.

13. The method of claim 12, wherein the compound competitively inhibits a P1B-type heavy metal ATPase.

14. The method of claim 13, wherein the P1B-type heavy metal ATPase comprises a P1B-type copper ATPase, and wherein the compound specifically binds to an intramembraneous pocket of the P1B-type copper ATPase, or wherein competitive inhibition of the P1B-type heavy metal ATPase comprises inhibition of copper-dependent tyrosinase enzyme activity in cells, thereby inhibiting melanogenesis.

15. The method of claim 14, wherein the P1B-type copper ATPase comprises ATP7A and/or ATP7B, wherein the compound competitively inhibits ATP7A and/or ATP7B by blocking entry of copper into ATP7A and/or ATP7B, and optionally wherein the compound disrupts transmembrane copper transport.

16. The method of claim 15, wherein competitive inhibition of ATP7A disrupts delivery of copper to at least one lysyl oxidase (LOX), thereby inhibiting activity of the at least one LOX, or wherein competitive inhibition of ATP7A and/or ATP7B comprises inhibition of LOX or LOXL1-4 enzyme activity.

17. The method of claim 12, wherein the therapeutically effective amount of the compound or the pharmaceutical composition is from about 10 nM to about 1 mM, and wherein the therapy is administered by oral administration, transdermal administration, topical administration, ocular administration, sublingual administration, parenteral administration, aerosol administration, administration via inhalation, intravenous or intra-arterial administration, local administration via injection or cannula, vaginal administration, and/or rectal administration.

18. (canceled)

19. The method of claim 12, wherein the health condition and/or disease comprises cancer and/or disease with a fibrotic component.

20-21. (canceled)

22. The method of claim 19, wherein the health condition and/or disease is a disease with a fibrotic component and wherein the disease with a fibrotic component is tissue scarring, pulmonary fibrosis, hepatic fibrosis, kidney fibrosis, heart fibrosis, skin fibrosis, scleroderma, systemic sclerosis, or primary sclerosing cholangitis, or wherein the health condition and/or disease is Menkes disease, Wilson disease, Alzheimer's disease, Amyotrophic lateral sclerosis, Parkinson's disease, Huntington's disease, Creutzfeldt Jakob disease, MEDNIK syndrome, post inflammatory hyperpigmentation, melasma, solar lentigines (sun spots), ephelides (freckles), café au lait macules, vitiligo, pityriasis alba, tinea versicolor, or post inflammatory hypopigmentation, or wherein the cancer is carcinoma, blood cancer, sarcoma, mesothelioma, colorectal cancer, pancreatic cancer, head and neck cancer, skin cancer, gastric cancer, breast cancer, prostate cancer, thyroid cancer, endometrial cancer, ovarian cancer, lung cancer, hepatocellular carcinoma, or kidney renal papillary cell carcinoma, and optionally wherein the head and neck cancer is esophageal cancer and wherein the pancreatic cancer is pancreatic ductal adenocarcinoma.

23-24. (canceled)

25. The method of claim 12, wherein the compound is administered to prevent and/or treat microbial growth or infection or microbial resistance to silver and/or copper, and wherein if the compound is administered with a therapeutically effective amount of silver and/or copper, the compound augments bactericidal or fungicidal properties of the silver and/or copper.

26-36. (canceled)
